(19) United States
(12) Patent Application Publication
(10) Pub. No.: US 2025/0037828 A1
(43) Pub. Date: Jan. 30, 2025

(54) MACHINE LEARNING FOR REAL-TIME DECISION SUPPORT

(71) Applicant: PRXCISION, Inc., Schenectady, NY (US)

(72) Inventors: Paul G. AMBROSE, Schenectady, NY (US); Sujata BHAVNANI, Schenectady, NY (US); Michael GILBERT, Schenectady, NY (US); Christopher M. RUBINO, Schenectady, NY (US)

(21) Appl. No.: 18/753,444

(22) Filed: Jun. 25, 2024

Related U.S. Application Data
(63) Continuation-in-part of application No. 17/575,905, filed on Jan. 14, 2022.
Application No. 17/575,905 is a continuation of application No. 16/740,913, filed on Jan. 13, 2020, now Pat. No. 11,227,680.
Application No. 16/740,913 is a continuation-in-part of application No. 14/600,948, filed on Jan. 20, 2015, now Pat. No. 10,534,895.
(60) Provisional application No. 63/510,950, filed on Jun. 29, 2023.

(51) Int. Cl. G16H 20/10; G06F 3/0482; G06F 3/04847; G16H 10/40; G16H 10/60; G16H 20/30; G16H 50/50; G16H 70/20; G16H 70/60

(52) U.S. Cl. CPC G16H 20/10; G06F 3/0482; G06F 3/04847; G16H 10/40; G16H 10/60; G16H 20/30; G16H 50/50; G16H 70/20; G16H 70/60

(57) ABSTRACT

A computer system, computer program product and method for determining a probability of attaining a PK-PD target associated with efficacy for a patient that includes a processor(s) determining that a trigger event related to a patient, wherein the patient record comprises an order for a current drug regimen, has occurred in an electronic health record (EHR) system communicatively coupled to the one or more processors. The processor obtains descriptive information relating to a patient. The processor selects a pharmacokinetic model. The processor applies the pharmacokinetic model and utilizes the information relating to the patient to determine, for each of the one or more drug therapies, a probability of attaining a PK-PD target associated with efficacy for the patient with the infection. The processor generates rankings for the drug therapies and determines if the current drug regimen comprises a probability above a threshold. The processor generates a new order.

### CROSS REFERENCE TO RELATED APPLICATIONS

This application claims priority to U.S. Provisional Patent Application No. 63/510,950, entitled “SYSTEM AND METHOD FOR RANKING OPTIONS FOR MEDICAL TREATMENTS,” which was filed on Jun. 29, 2023. This application is also a continuation-in-part of U.S. application Ser. No. 17/575,905, entitled “SYSTEM AND METHOD FOR RANKING OPTIONS FOR MEDICAL TREATMENTS,” which was filed on Jan. 14, 2022, which is a continuation-in-part of U.S. application Ser. No. 16/740,913, entitled “SYSTEM AND METHOD FOR RANKING OPTIONS FOR MEDICAL TREATMENTS,” filed Jan. 13, 2020, which is a continuation-in-part of U.S. application Ser. No. 14/600,948, entitled “SYSTEM AND METHOD FOR RANKING OPTIONS FOR MEDICAL TREATMENTS,” filed Jan. 20, 2015. These applications are all hereby incorporated herein by reference in their entireties for all purposes.

### BACKGROUND OF INVENTION

The invention relates generally to systems and methods for utilizing machine learning to provide real-time decision support, for example, to enable health care providers to discriminate among potential anti-infective therapies for the treatment of selected infectious diseases.

Artificial intelligence (AI) refers to intelligence exhibited by machines. Artificial intelligence (AI) research includes search and mathematical optimization, neural networks, and probability. Artificial intelligence (AI) solutions involve features derived from research in a variety of different science and technology disciplines ranging from computer science, mathematics, psychology, linguistics, statistics, and neuroscience. Machine learning has been described as the field of study that gives computers the ability to learn without being explicitly programmed.

The goal of anti-infective stewardship is to select therapies that optimize the probability of positive outcomes for patients suffering from an infection. The primary focus of anti-infective stewardship is the optimal selection of anti-infective therapy, including dose, dosing interval, and duration. Due to the emergence of anti-infective-resistant pathogens, selecting optimal anti-infective therapy is more complex than at any other time since the advent of penicillin.

The correct therapy that optimizes the probability of a positive outcome can be impacted based on various changes being made within one or more interconnected electronic medical record systems.

### SUMMARY OF INVENTION

Shortcomings of the prior art are overcome and additional advantages are provided through the provision of a method for providing real-time decision support for an electronic health record (EHR) system, the method includes: determining, by one or more processors, that a trigger event related to a patient, wherein the patient record comprises an order for a current drug regimen, has occurred in an electronic health record (EHR) system communicatively coupled to the one or more processors; based on obtaining the notification, obtaining, by the one or more processors, from one or more electronic medical records (EMRs) stored in the EHR system communicatively coupled to the one or more processors, descriptive information relating to a patient, the descriptive information comprising one or more data elements selected from the group consisting of: an infection acquired by the patient, a pathogen isolated from the patient, a creatinine clearance of the patient, a weight of the patient, and a height of the patient; based on the one or more drug therapies, selecting a pharmacokinetic model; applying, by the one or more processors, the pharmacokinetic model and utilizing the information relating to the patient to determine, for each of the one or more drug therapies, a probability of attaining a PK-PD target associated with efficacy for the patient with the infection; automatically generating, by the one or more processors, rankings, for each of the one or more drug therapies, by ordering each probability of attaining the PK-PD target associated with efficacy for the patient with the infection, for each of the one or more drug therapies, for the one or more drug therapies, wherein the rankings comprise a ranked list with the probability of attaining a PK-PD target associated with efficacy for the patient with the infection for each of the one or more drug therapies, ranked in order of predicted efficacy; determining, by the one or more processors, based on the rankings, if the current drug regimen comprises a probability above a preconfigured threshold; and based on the determining and the rankings, generating a recommendation for a new order.

Shortcomings of the prior art are overcome, and additional advantages are provided through the provision of a computer program product for providing real-time decision support for an EHR system, the method includes. The computer program product comprises a storage medium readable by a one or more processors and storing instructions for execution by the one or more processors for performing a method. The method includes, for instance: determining, by one or more processors, that a trigger event related to a patient, wherein the patient record comprises an order for a current drug regimen, has occurred in an electronic health record (EHR) system communicatively coupled to the one or more processors; based on obtaining the notification, obtaining, by the one or more processors, from one or more electronic medical records (EMRs) stored in the EHR system communicatively coupled to the one or more processors, descriptive information relating to a patient, the descriptive information comprising one or more data elements selected from the group consisting of: an infection acquired by the patient, a pathogen isolated from the patient, a creatinine clearance of the patient, a weight of the patient, and a height of the patient; based on the one or more drug therapies, selecting a pharmacokinetic model; applying, by the one or more processors, the pharmacokinetic model and utilizing the information relating to the patient to determine, for each of the one or more drug therapies, a probability of attaining a PK-PD target associated with efficacy for the patient with the infection; automatically generating, by the one or more processors, rankings, for each of the one or more drug therapies, by ordering each probability of attaining the PK-PD target associated with efficacy for the patient with the infection, for each of the one or more drug therapies, for the one or more drug therapies, wherein the rankings comprise a ranked list with the probability of attaining a PK-PD target associated with efficacy for the patient with the infection for each of the one or more drug therapies, ranked in order of predicted efficacy; determining, by the one or more processors, based on the rankings, if the current drug regimen comprises a probability above a preconfigured threshold; and based on the determining and the rankings, generating a recommendation for a new order.

Shortcomings of the prior art are overcome and additional advantages are provided through the provision of a system for providing real-time decision support for an EHR system, the system includes: a memory; and one or more processors in communications with the memory, wherein the computer system is configured to perform a method, the method including: determining, by the one or more processors, that a trigger event related to a patient, wherein the patient record comprises an order for a current drug regimen, has occurred in an electronic health record (EHR) system communicatively coupled to the one or more processors; based on obtaining the notification, obtaining, by the one or more processors, from one or more electronic medical records (EMRs) stored in the EHR system communicatively coupled to the one or more processors, descriptive information relating to a patient, the descriptive information comprising one or more data elements selected from the group consisting of: an infection acquired by the patient, a pathogen isolated from the patient, a creatinine clearance of the patient, a weight of the patient, and a height of the patient; based on the one or more drug therapies, selecting a pharmacokinetic model; applying, by the one or more processors, the pharmacokinetic model and utilizing the information relating to the patient to determine, for each of the one or more drug therapies, a probability of attaining a PK-PD target associated with efficacy for the patient with the infection; automatically generating, by the one or more processors, rankings, for each of the one or more drug therapies, by ordering each probability of attaining the PK-PD target associated with efficacy for the patient with the infection, for each of the one or more drug therapies, for the one or more drug therapies, wherein the rankings comprise a ranked list with the probability of attaining a PK-PD target associated with efficacy for the patient with the infection for each of the one or more drug therapies, ranked in order of predicted efficacy; determining, by the one or more processors, based on the rankings, if the current drug regimen comprises a probability above a preconfigured threshold; and based on the determining and the rankings, generating a recommendation for a new order.

In some examples of the computer-implemented method, computer program product and/or the system, the program code determining if the current drug regimen comprises the probability above the preconfigured threshold comprises the program code determining that the current drug regimen comprises the probability above the preconfigured threshold, and the generating comprises: the program code generating a clinical note of a recommendation, where the clinical note of a recommendation comprises the new order, and where the new order is an order for the current drug regimen, and the program code transmitting the clinical node of the recommendation to the EHR system communicatively coupled to the one or more processors.

In some examples of the computer-implemented method, computer program product and/or the system, the program code determining if the current drug regimen comprises the probability above the preconfigured threshold comprises the program code determining that the current drug regimen does not comprise a probability above the preconfigured threshold. The program code generating including the program code generating a clinical note of a recommendation, where the clinical note of a recommendation comprises the new order, and the program code transmitting the clinical node of the recommendation to the EHR system communicatively coupled to the one or more processors.

In some examples of the computer-implemented method, computer program product and/or the system, the program code generating the recommendation for a new order comprises: the program code generating an unsigned medication order based on the ranked list, the unsigned medication order comprising the new order.

In some examples of the computer-implemented method, computer program product and/or the system, the program code generating the recommendation for a new order comprises: the program code transmitting an alert to at least one user, the program code obtaining a response to the alert, where the response comprises a selection of a designation of a drug therapy from the one or more drug therapies comprising the ranked list, where the drug therapy designated comprises the new order, and the program code generating an unsigned medication order comprising the new order.

In some examples of the computer-implemented method, computer program product and/or the system, the program code transmitting the alert comprises: the program code generating a message comprising a link to launch a graphical user interface, where the one or more processors automatically display the rankings in the graphical user interface upon selection of the link by a user receiving the message, and the program code transmitting the message to at least one user pre-defined to receive the message, where the user contact information is saved in a database communicatively coupled to the one or more processors, where the response the selection of the designation of a drug therapy is performed by the user in the graphical user interface.

In some examples of the computer-implemented method, computer program product and/or the system, the program code transmitting the alert comprises: the program code displaying the rankings, in a graphical user interface, where the response the selection of the designation of a drug therapy is performed by the user in the graphical user interface.

In some examples of the computer-implemented method, computer program product and/or the system, the trigger event comprises an update to at least one field of at least one electronic medical record in the EHR system.

In some examples of the computer-implemented method, computer program product and/or the system, the trigger event is selected from the group consisting of: entry of a prescription for a given antibiotic, reception of a culture with a given pathogen, and entry of data comprising additional information about an existing prescription.

In some examples of the computer-implemented method, computer program product and/or the system, the program code retains the recommendation on a memory device. The program code prompts, through a user interface, a user to provide data indicating an actual efficacy of the drug therapy as utilized by the patient with the infection at one or more predetermined intervals after obtaining the recommendation. The program code obtains, responsive to the prompting, the data indicating the actual efficacy of the drug therapy.

In some examples of the computer-implemented method, computer program product and/or the system, the program code generates or updates, based on data comprising the data indicating the actual efficacy, a base model, where the base model describes a relationship between given patient response and PK-PD target attainment that accounts based on patient-specific response modifiers.

In some examples of the computer-implemented method, computer program product and/or the system, the data further comprises data selected from the group consisting of: patient demographic data, clinical data, and laboratory data.

In some examples of the computer-implemented method, computer program product and/or the system, the program code selecting the pharmacokinetic models by: for each of the one or more drug therapies, the program code determining a class for a PK-PD index. Based on determining that a drug therapy of the one or more drug therapies is in a first class, the program code selects a pharmacokinetic model, where applying the pharmacokinetic model comprises evaluating total drug exposure in a 24-hour period, for the drug therapy, to determine the probability of attaining a PK-PD target associated with efficacy for the patient with the infection. Based on determining that a drug therapy of the one or more drug therapies is in a second class, the program code selects a pharmacokinetic model, where applying the pharmacokinetic model comprises evaluating % time above MIC, for the drug therapy, to determine the probability of attaining a PK-PD target associated with efficacy for the patient with the infection.

In some examples of the computer-implemented method, computer program product and/or the system, the program code obtains additional information identifying an infection. Based on the additional information, the program code generates and displays a second list comprising one or more pathogens consistent with the additional information. The program code obtains a first indication designating at least one pathogen from the second list comprising one or more pathogens from the second list. Based on at the obtaining of the least one pathogen from the second list, the program code generates a third list comprising one or more drug therapies utilized to treat the at least one pathogen. The program code obtains descriptive information relating to a second patient, the descriptive information comprising one or more data elements selected from the group consisting of: an infection acquired by the second patient, a pathogen isolated from the second patient, a creatinine clearance of the second patient, a weight of the second patient, and a height of the second patient. Based on the one or more drug therapies in the third list, the program code selects a given pharmacokinetic model. The program code applies the given pharmacokinetic model and utilizing the information relating to the second patient and the base model to determine, for each of the one or more drug therapies of the third list, a probability of attaining a PK-PD target associated with efficacy for the second patient with the infection. The program code automatically generates current rankings for each of the one or more drug therapies of the third list, by ordering each probability of attaining the PK-PD target associated with efficacy for the second patient with the infection, for each of the one or more drug therapies of the third list, for the one or more drug therapies of the third list, where the current rankings comprise a ranked list with the probability of attaining a PK-PD target associated with efficacy for the second patient with the infection for each of the one or more drug therapies of the third list, ranked in order of predicted efficacy.

In some examples of the computer-implemented method, computer program product and/or the system, the patient-specific response modifiers are selected from the group consisting of: previous antibiotic use, age, and clearing organ function.

In some examples of the computer-implemented method, computer program product and/or the system, the program code determining that the trigger event has occurred comprises: the program code monitoring, the program code logging of changes to the EHR system, and the program code determining, based on the logging, that the trigger event has occurred.

In some examples of the computer-implemented method, computer program product and/or the system, the program code determining that the trigger event has occurred comprises: the program code obtains from an application programming interface communicatively coupled with the EHR system, a notification that the trigger event has occurred.

Computer systems, computer program products and methods relating to one or more aspects of the technique are also described and may be claimed herein. Further, services relating to one or more aspects of the technique are also described and may be claimed herein.

Additional features are realized through the techniques of the present invention. Other embodiments and aspects of the invention are described in detail herein and are considered a part of the claimed invention.

### BRIEF DESCRIPTION OF DRAWINGS

One or more aspects are particularly pointed out and distinctly claimed as examples in the claims at the conclusion of the specification. The foregoing and objects, features, and advantages of one or more aspects are apparent from the following detailed description taken in conjunction with the accompanying drawings in which:

FIG. 1 depicts one example of an aspect of a computing environment used to execute one or more aspects of an embodiment of the present invention;

FIG. 2 depicts one embodiment of a single processor computing environment to incorporate and use one or more aspects of the present invention;

FIG. 3 depicts one embodiment of a computer program product incorporating one or more aspects of the present invention;

FIG. 4 depicts a workflow of an embodiment of the present invention;

FIGS. 5-20 depict examples of an exemplary graphical user interface (GUI) produced by an aspect of the present invention;

FIG. 21 depicts a workflow of an embodiment of the present invention;

FIG. 22 depicts a model related to an aspect of an embodiment of the present invention; and

FIG. 23 depicts an example of a two-compartment model that is utilized when evaluating meropenem.

FIG. 24 is a workflow that illustrates various aspects of some embodiments of the present invention.

FIG. 25 is a workflow that illustrates various aspects of some embodiments of the present invention.

FIGS. 27-30C are examples of graphical user interfaces generated by the program code to prompt a user to provide information and to provide results to the user.

FIG. 31 depicts an example of a workflow illustrating various aspects of some embodiments of the present invention.

FIG. 32 depicts an example of a workflow illustrating various aspects of some embodiments of the present invention.

FIG. 33 depicts an example of a workflow illustrating various aspects of some embodiments of the present invention.

FIG. 34 depicts an example of a workflow illustrating various aspects of some embodiments of the present invention.

FIG. 35 depicts an example of a workflow illustrating various aspects of some embodiments of the present invention.

FIG. 36 depicts an example of a workflow illustrating various aspects of some embodiments of the present invention.

FIG. 37 depicts an example of a workflow illustrating various aspects of some embodiments of the present invention.

FIG. 38 depicts an example of a workflow illustrating various aspects of some embodiments of the present invention.

FIG. 39 depicts an example of a workflow illustrating various aspects of some embodiments of the present invention.

FIG. 40 depicts an example of a workflow illustrating various aspects of some embodiments of the present invention.

FIG. 41 depicts an example of a workflow illustrating various aspects of some embodiments of the present invention.

FIG. 42 depicts an example of a workflow illustrating various aspects of some embodiments of the present invention.

### DETAILED DESCRIPTION OF THE INVENTION

The accompanying figures, in which like reference numerals refer to identical or functionally similar elements throughout the separate views and which are incorporated in and form a part of the specification, further illustrate the present invention and, together with the detailed description of the invention, serve to explain the principles of the present invention. As understood by one of skill in the art, the accompanying figures are provided for ease of understanding and illustrate aspects of certain embodiments of the present invention. The invention is not limited to the embodiments depicted in the figures.

As understood by one of skill in the art, program code, as referred to throughout this application, includes both software and hardware. For example, program code in certain embodiments of the present invention includes fixed function hardware, while other embodiments utilized a software-based implementation of the functionality described. Certain embodiments combine both types of program code.

Aspects of the present invention and certain features, advantages, and details thereof, are explained more fully below with reference to the non-limiting examples illustrated in the accompanying drawings. Descriptions of well-known materials, fabrication tools, processing techniques, etc., are omitted so as not to unnecessarily obscure the invention in detail. It should be understood, however, that the detailed description and the specific examples, while indicating aspects of the invention, are given by way of illustration only, and not by way of limitation. Various substitutions, modifications, additions, and/or arrangements, within the spirit and/or scope of the underlying inventive concepts will be apparent to those skilled in the art from this disclosure.

The terms “connect,” “connected,” “contact” “coupled” and/or the like are broadly defined herein to encompass a variety of divergent arrangements and assembly techniques. These arrangements and techniques include, but are not limited to (1) the direct joining of one component and another component with no intervening components therebetween (e.g., the components are in direct physical contact); and (2) the joining of one component and another component with one or more components therebetween, provided that the one component being “connected to” or “contacting” or “coupled to” the other component is somehow in operative communication (e.g., electrically, fluidly, physically, optically, etc.) with the other component (notwithstanding the presence of one or more additional components therebetween). It is to be understood that some components that are in direct physical contact with one another may or may not be in electrical contact and/or fluid contact with one another. Moreover, two components that are electrically connected, electrically coupled, optically connected, optically coupled, fluidly connected or fluidly coupled may or may not be in direct physical contact, and one or more other components may be positioned therebetween.

The terms “including” and “comprising”, as used herein, mean the same thing.

The terms “substantially”, “approximately”, “about”, “relatively,” or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, they can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to 0.1%, such as less than or equal to ±0.05%. If used herein, the terms “substantially”, “approximately”, “about”, “relatively,” or other such similar terms may also refer to no fluctuations.

As used herein, “electrically coupled” refers to a transfer of electrical energy between any combination of a power source, an electrode, a conductive surface, a droplet, a conductive trace, wire, waveguide, nanostructures, other circuit segment and the like. The terms electrically coupled may be utilized in connection with direct or indirect connections and may pass through various intermediaries, such as a fluid intermediary, an air gap and the like.

As used herein, “neural networks” refer to a biologically inspired programming paradigm which enables a computer to learn from observational data. This learning is referred to as deep learning, which is a set of techniques for learning in neural networks. Neural networks, including modular neural networks, are capable of pattern recognition with speed, accuracy, and efficiency, in situations where data sets are multiple and expansive, including across a distributed network of the technical environment. Modern neural networks are non-linear statistical data modeling tools. They are usually used to model complex relationships between inputs and outputs or to identify patterns in data (i.e., neural networks are non-linear statistical data modeling or decision-making tools). In general, program code utilizing neural networks can model complex relationships between inputs and outputs and identify patterns in data. Because of the speed and efficiency of neural networks, especially when parsing multiple complex data sets, neural networks and deep learning provide solutions to many problems in image recognition, speech recognition, and natural language processing. Neural networks can model complex relationships between inputs and outputs to identify patterns in data, including in images, for classification.

As used herein, a “convolutional neural network” (CNN) is a class of neural network. CNNs utilize feed-forward artificial neural networks and are most commonly applied to analyzing visual imagery. CNNs are so named because they utilize convolutional layers that apply a convolution operation (a mathematical operation on two functions to produce a third function that expresses how the shape of one is modified by the other) to the input, passing the result to the next layer. The convolution emulates the response of an individual neuron to visual stimuli. Each convolutional neuron processes data only for its receptive field. It is not practical to utilize general (i.e., fully connected feedforward) neural networks to process images, as very high number of neurons would be necessary, due to the very large input sizes associated with images. Utilizing a CNN addresses this issue as it reduces the number of free parameters, allowing the network to be deeper with fewer parameters, as regardless of image size, the CNN can utilize a consistent number of learnable parameters because CNNs fine-tune large amounts of parameters and massive pre-labeled datasets to support a learning process. CNNs resolve the vanishing or exploding gradients problem in training traditional multi-layer neural networks, with many layers, by using backpropagation. Thus, CNNs can be utilized in large-scale (image) recognition systems, giving state-of-the-art results in segmentation, object detection and object retrieval. CNNs can be of any number of dimensions, but most existing CNNs are two-dimensional and process single images. These images contain pixels in a two-dimensional (2D) space (length, width) that are processed through a set of two-dimensional filters to understand what set of pixels best correspond to the final output classification. A three-dimensional CNN (3D-CNN) is an extension of the more traditional two-dimensional CNN and a 3D-CNN is typically used in problems related to video classification. 3D-CNNs accept multiple images, often sequential image frames of a video, and use 3D filters to understand the 3D set of pixels that are presented to it. In the present context, as discussed herein, images provided to a CNN include images of a culture, including but not limited to, stain images of a culture.

As used herein, a “classifier” is comprised of various cognitive algorithms, artificial intelligence (AI) instruction sets, and/or machine learning algorithms. Classifiers can include, but are not limited to, deep learning models (e.g., neural networks having many layers) and random forests models. Classifiers classify items (data, metadata, objects, etc.) into groups, based on relationships between data elements in the metadata from the records. In some embodiments of the present invention, the program code can utilize the frequency of occurrences of features in mutual information to identify and filter out false positives. In general, program code utilizes a classifier to create a boundary between data of a first quality data of a second quality. As a classifier is continuously utilized, its accuracy can increase as testing the classifier tunes its accuracy. When training a classifier, in some examples, program code feeds a pre-existing feature set describing features of metadata and/or data into the one or more cognitive analysis algorithms that are being trained. The program code trains the classifier to classify records based on the presence or absence of a given condition, which is known before the tuning. The presence or absence of the condition is not noted explicitly in the records of the data set. When classifying a source as providing data of a given condition (based on the metadata), utilizing the classifier, the program code can indicate a probability of a given condition with a rating on a scale, for example, between 0 and 1, where 1 would indicate a definitive presence. The classifications need not be binary and can also be values in an established scale.

As used herein, the term “deep learning model” refers to a type of classifier. A deep learning model can be implemented in various forms such as by a neural network (e.g., a convolutional neural network). In some examples, a deep learning mode includes multiple layers, each layer comprising multiple processing nodes. In some examples, the layers process in sequence, with nodes of layers closer to the model input layer processing before nodes of layers closer to the model output. Thus, layers feed to the next. Interior nodes are often “hidden” in the sense that their input and output values are not visible outside the model.

As used herein, the term “processor” refers to a hardware and/or software device that can execute computer instructions, including, but not limited to, one or more software processors, hardware processors, field programmable gate arrays (FPGAs), application-specific integrated circuits (ASICs), and/or programmable logic devices (PLDs).

As used herein an electronic medical record (EMR) system is computer system that houses, updates, creates, manages deletes, etc., one or more electronic records of health-related information on individuals that can be created, gathered, managed, and consulted by authorized clinicians and staff within at least one health care organization. Generally, EMR systems are understood to provide substantial benefits to physicians, clinic practices, and health care organizations in part because these systems can facilitate workflow and improve the quality of patient care and patient safety. There has been a lag to adoption of EMRs within the certain countries on one barrier is a failure to redesign clinical process and workflow to incorporate the technology systems. As discussed herein, embodiments of the present invention utilize various unique workflows to optimize benefits provided by EMRs.

As used herein, an electronic health record (EHR) is the systematized collection of patient and/or population electronically stored health information in a digital format. An EHR for a given patient or population can be shared across different health care settings. The terms EMR system and EHR system are used interchangeably throughout. An example of EHR systems that are compatible with aspects of the examples described herein are Epic and Cerner, Epic and Cerner both the Health Level Seven International (HL7) specifications to format their messages. HL7 is currently the international standard for transferring clinical and administrative data between software applications.

As used herein, the term “antibiogram” is an overall profile of antimicrobial susceptibility testing results of a specific microorganism to a battery of antimicrobial drugs. In the context of the examples here an antibiogram provides a listing of bugs down the left (y axis) and antibiotics across the top (x-axis), with % susceptible as the data (% of total isolates that got an S—or could be S and I) and total number of isolates for each bug. This listing represents aggregation of all of the C&S (culture and sensitivity) lab reports (cultures) over the previous 6 months or a year (or another interval). The listing includes a total number of isolates for each bug which indicate total incidence (rules can remove duplicate results from the same patient so results are not double counted for the same organism from the same patient, but results could potentially double count sputum vs blood cultures from the same patient, for example). The antibiogram does not include, in the examples herein, types of infections. About 95%-98% of hospitals in the US have a hospital antibiogram, ˜80% may only have a single hospital-wide antibiogram, the remaining 20% of hospitals with an antibiogram may separate out isolates that were taken in the ICU, or Outpatients for example—these may be organized separately to create an “ICU Antibiogram” or “Outpatient Antibiogram”. An antibiogram is used to help AMS Pharmacists and ID doctors to coax prescribers to stop ordering antibiotics where the bugs have shown to be resistant to that drug and thus antibiograms help an empiric therapy to determine what works locally. In some examples herein, the program code in embodiments of the present invention can import an antibiogram from the hospital. As will be discussed in greater detail herein, various embodiments of the present invention enable authorized users to edit an existing antibiogram in an interface generated by the program code.

As used herein, the term Fast Healthcare Interoperability Resources (FHIR) and standards describing data formats, elements, and an application programming interface for exchanging electronic health records.

As used herein, representational state transfer (REST) or RESTful web services comprise program code that provides interoperability between computer systems on the Internet or other private networks. REST-compliant web services enable a requestor to access and manipulate representations of web resources (e.g., applications) using a uniform and predefined set of stateless operations. A REST API uses generally HTTP requests to GET, PUT, POST and DELETE data and relies on a stateless, client-server, cacheable communications protocol. REST is an architecture style for designing networked applications and is therefore particularly prevalent in and relevant to, multi-server (multi-resource) computing environments. Specifically, because APIs provide interoperability between computer systems and allow for standardized connectivity, they are frequently utilized as endpoints on servers that enable other resources to access applications associated with the APIs that are deployed on the servers. For example, various REST APIs may be available from each of the individual servers in a multi-server environment, such as a cloud computing environment, providing endpoints to applications executing on the various servers.

Embodiments of the present invention, include a computer-program product, a computer system, and a computer-implemented method that include provide a service-based software that interfaces with an EMR or EHR system to provide real-time recommendations regarding treatments for medical conditions. In embodiments of the present invention, program code continuously analyzes information in an EMR system to tune various treatment parameters, including based on triggering events within the EMR system. Triggering events comprise actions within the system that comprise changes to data. To that end, in some examples, the program code (e.g., software) runs as a service and provide recommendations, in real-time, to various types of users of the EMR system, utilizing different graphical user interfaces and other interfaces to interact with the system. The interconnectivity of the system within a given technical architecture enables the recommendations to be impacted by various triggering events. These triggering events include, but are not limited to, updates to various aspects of data in the EMR system from various sources, including different types of users and systems with access to the EMRs. In some examples herein, program code (running continuously as a service) automatically analyzes updates (and other types of changes implemented within the system, which are referred to herein as triggering events), and determines whether the updates are material to medical treatments being provided to at least one patient. In the event that the program code determines that a given update is material, the program code can automatically gather additional data and apply various analyses and simulations to the data. Based on the results of the analyses and/or simulations, the program code determines whether to proceed with an action. An action can include, but is not limited to, an electronic communication. The program code also determines the content of the communication and to whom and how the update should be communicated. In various examples, recipients of communications and the methods utilized to transmit the communications can be pre-configured or dynamically determined, by the program code, based on various attributes of the content of the communication.

Embodiments of the present invention provide various advantages over existing systems because, as will explained in more detail below, embodiments of the present invention include software that can run as a background process and complete the following tasks (as a non-limiting example): 1) communicate with an existing EHR system; 2) listen for events based on the connectivity to the EHR system; 3) collect information to make various decisions based on various calculations; and 4) present a recommendation for a given issue. Thus, the program code can identify an event that could raise an issue, identify the information to evaluate this issue, implement a process to determine whether the change comprises a covered event, and automatically instigate a pre-determined response to the covered event. For example, in some embodiments of the present invention, the program can determine if a treatment change is potentially warranted and whether designated individuals should be alerted to the change. Changes can include but are not limited to, evaluating, based on determining that a covered event has occurred, that a different drug regimen (and/or a combination of regimens) could be warranted/recommended. The program code can also manage competing priorities when making recommendations, based on a wholistic analysis of the diverse data. Embodiments of the present invention provide a practical application in clinical practices at least because the program code can apply a PK-PD model to determine dosages, drug regimens, and/or prioritization of different approaches.

Covered events are events in which the program code determines, based on monitoring/listening, that it should evaluate whether to take an action. In the context of the examples provided herein, covered events can include, but are not limited to, a new antibiotic prescription, a new culture result, an updated culture result with MICs, a new rapid diagnostic result, a new drug concentration result, a new serum creatinine result (such that Creatinine Clearance changes+/−[20%]), anew gram stain result, and/or a new diagnosis. In certain examples herein, a covered events occurs when at least one of the following occurs: a patient is currently on or has a prescribed order for a covered antibiotic, a patient has a covered diagnosis, a new lab results, and/or diagnostic reports are available for the patient containing a covered pathogen. Covered events are configurable in various examples of the present invention. For example, covered pathogens, covered medications and diagnosis are configurable in various embodiments of the present invention.

Appropriate treatment with anti-infective therapies, including but not limited to, antibiotics, antibacterial, antifungals, antivirals, and/or antimicrobials involves many factors that cannot be controlled by clinicians. For example, factors such as inter-patient variability in drug exposure, the minimum inhibitory concentration (MIC) of the infecting pathogen, and the patient's clinical status, can affect the probability of attaining a pharmacokinetic-pharmacodynamic (PK-PD) target associated with efficacy for a drug regimen. The MIC refers to the minimum concentration of a drug therapy that will inhibit the growth of the isolated pathogen. Despite these uncertainties, embodiments of the present method and system enable a clinician (user) to obtain estimates of the probability of attaining PK-PD targets associated with efficacy in the context of predefined factors based upon the selection and application of pharmacokinetic models and simulation by program code executed on at least one processor of a computer system. To describe the concentration of drug over time in the body, pharmacokinetic models can be used to describe the disposition of a drug including where and how fast the drug is transferring throughout the body. As discussed below, embodiments of the present invention provide significantly more than existing approaches to providing drug therapy recommendations.

In an embodiment of the present invention, the predefined factors that enable the present technique to estimate probability of attaining a PK-PD target associated with efficacy outcome include, but are not limited to, factors that are within the control of the clinician and/or known to the clinician. The functionality described herein that enables the program code to estimate probability of attaining a PK-PD target associated with efficacy outcome is referred to as a PK-PD compass.

Embodiments of the present invention estimate anti-infective drug exposure for a given patient using data including, but not limited to, infection(s) acquired by the given patient, pathogen(s) isolated from the given patient, and demographic information describing the given patient, including but not limited to, the patient's creatinine clearance, weight, and height. The present invention obtains inputs and identifies and applies relevant pharmacokinetic models and/or tabular outputs to create a listing of potentially useful drug therapies. In embodiments of the present invention, results of the present technique include different options for antibiotic dosing regimens (which consider drug, dose and the dosing interval) for a given patient including drug, dose, and the dosing interval and a comparison of these different options with a ranking based on the probability of attaining PK-PD targets associated with efficacy. An embodiment of the present invention is designed to provide information rather than recommendations for individual patients. The information provided, including but not limited to, the options, may be utilized for decision support and not as a final recommendation without clinical judgment (i.e., without the consideration of other factors such as adverse events).

In an embodiment of the present invention, upon obtaining information related to the given person, for each drug therapy considered, the invention indexes drug exposure to a measure of susceptibility, the MIC, which represents the concentration of drug that inhibits the growth of the pathogen being considered. The MIC can either be a known value, a distribution of values, or the value of defining susceptibility based on in vitro susceptibility test interpretive criteria. In this embodiment, the indexed drug exposure for each drug, which is referred to as a PK-PD index, can take several forms, including but not limited to the following: the ratio of the area under the concentration time-curve over a period of time (e.g., 24 hours) to the MIC (AUC:MIC ratio), the percent of the dosing interval that the drug concentration remains above the MIC (% time above MIC), and the ratio of the maximal drug concentration in the dosing interval to the MIC (Cmax:MIC ratio). The PK-PD index for a given drug and dosing regimen is compared to that required for efficacy, based on pre-clinical or clinical infection exposure-response models. Using one or more equations and/or models that account for sources of variability, the probability of attaining a PK-PD index relative to those associated with efficacy based on pre-clinical or clinical infection exposure-response models (i.e., PK-PD targets associated with efficacy) for each listed antibiotic and dose regimen is then determined for that patient.

In an embodiment of the present invention, the software can determine a ranking for each evaluated drug therapy based on the probability of attaining a PK-PD target associated with efficacy relative to other identified relevant therapies.

In an embodiment of the present invention, collected information and resulting probabilities are stored for future access, for example, in a data store or a database that is accessible to program code executing on a processor in an embodiment of the present invention.

In a further embodiment of the present invention, a user can utilize the software to track results after an option is relayed to a given individual. In an embodiment of the present invention, the program code utilizes the patient information and the relevant data to estimate the probability of attaining a PK-PD target associated with efficacy for a given drug regimen. To provide the user with a full view of treatment options, in an embodiment of the present invention, in addition to evaluating the anti-infective used by the program code, the program code also identifies additional anti-infectives for consideration based on the patient information and/or relevant data. The one or more anti-infective obtained by the program code from the user as well as the additional anti-infectives may both be considered by the program code when estimating the probability of attaining a PK-PD target associated with efficacy for a given patient.

In an embodiment of the present invention, program code executing on one or more processors utilizes patient outcome data to train a machine learning data model to predict outcomes for new patients based on past outcomes. In embodiments of the present invention, the program code utilizes aggregate patient demographic, clinical, laboratory and outcome data to construct a base model. The base model describes a relationship between patient response and PK-PD target attainment that accounts for patient-specific response modifiers (e.g., previous antibiotic use, age, clearing organ function, etc.). Thus, with each new patient, the program code utilizes (as explained herein) that new patient's data will be used to estimate PK-PD target attainment and its associated the probability. As discussed herein, the program code ranks (orders) the results (positive responses) and provides the results to a user through a graphical user interface. Responsive to obtaining the ranking a user selects a regimen for the patient. The selection of the ranked results can be based on clinical judgment of a care provider. However, patient response to the selected regimen can be monitored. The monitoring by the program code (e.g., of medical records to obtain data, including but not limited patient response, PK-PD target attainment, patient-specific response modifiers) is utilized by the data to modify the base model. Thus, the base model is continuously improved through this machine learning.

Embodiments of the present invention are inextricably linked to computing and comprise a practical application. Regarding being inextricably linked to computing, embodiments of the present invention utilize the immediacy provided by computing and network communications as well as machine learning to automatically generate rankings for drug therapies. In embodiments of the present invention, program code determined relevant drug therapies (in accordance with the details described herein) and orders each relevant therapy by probability of attaining the PK-PD target associated with efficacy for a given patient with an infection. These rankings are displayed in order of predicted efficacy. However, the program code continually improves the accuracy of the results through machine learning. Specifically, the program code can machine learn from the displayed results by obtaining a designation of a drug therapy from the therapies displayed, and retaining, the designation on a memory device. The program code can continue to obtain information related to a patient being treated with the drug therapy (patient response, PK-PD target attainment, patient-specific response modifiers) and utilize this data to train the model. The program code retains this data in the memory device (e.g., one or more memory devices). Thus, the program code can continue to automatically provide results to users, with improved efficacy. This machine learning, for example, is inextricably linked to computing. However, the immediacy of the data analyses and calculation and display of results is likewise inextricably linked to computing because the management of the data and coherence and immediacy of the response is enabled through computing technology. Additionally, embodiments of the present invention provide a practical application at least because the program code provides practical results, rankings for different regimens for a given patient, with increasing accuracy. Embodiments of the present invention are additionally not abstract based on the particularity of data elements utilized as well as the tangible results generated by the program code. For example, in some embodiments of the present invention the program code obtains descriptive information that includes data elements, including but not (always) limited to, an infection acquired by the patient, a pathogen isolated from the patient, a creatinine clearance of the patient, a weight of the patient, and a height of the patient. This data is utilized by the program code, in embodiments of the present invention, to automatically generate and provide the aforementioned results.

Embodiments of the present invention comprise a practical application for a number of reasons, some of which are discussed above. However, as another example, program code in some embodiments of the present invention, executing on one or more processors, applies a pharmacokinetic model and utilizes information relating to a patient to determine, for various (determined to be relevant) drug therapies, a probability of attaining a PK-PD target associated with efficacy for the patient with the infection. The program code also automatically generates rankings for each of the drug therapies, by ordering each probability of attaining the PK-PD target associated with efficacy for the patient with the infection, for each of the drug therapies. The program code also displays the rankings, which comprise a ranked list with the probability of attaining a PK-PD target associated with efficacy for the patient with the infection for each of the drug therapies, ranked in order of predicted efficacy. The program code then obtains a designation of a drug therapy from the drug therapies displayed and retains the designation on a memory device. These aspects are all practical applications.

In addition to making an initial determination of a probability of attaining a PK-PD target associated with efficacy for the patient with the infection, in embodiments of the present invention, the program code can continue to run as a service and thus, when a change is implemented in the EMR, the program code can determine whether to recommend treatment of other changes based on this change. As discussed above, the changes implemented within the system that trigger the program code to gather data and make calculations, including re-calculating PK-PD values, are referred to as trigger events. Embodiments of the present invention are inextricably linked to computing and comprise a practical application also because based on the interconnectivity of the program code to an EMR system and the program code constantly monitoring the system and the EMRs contained within the system, the program code can determine when a trigger event has occurred, determine whether he event is material, and based on determining that it is material, can provide notifications with recommendations for treatment. Thus, the program code can take advantage of the aforementioned model and continually train and update the model as it is utilized each time a trigger event occurs. The utility of the program code and the model increase with use and thus, implementing this trigger event reaction feature and implementing the program code as a service enhances the aforementioned practical application discussed above.

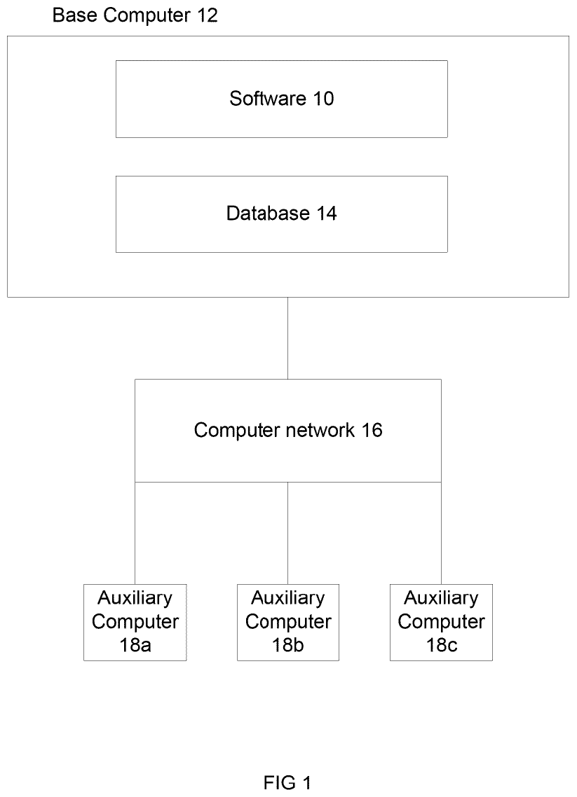

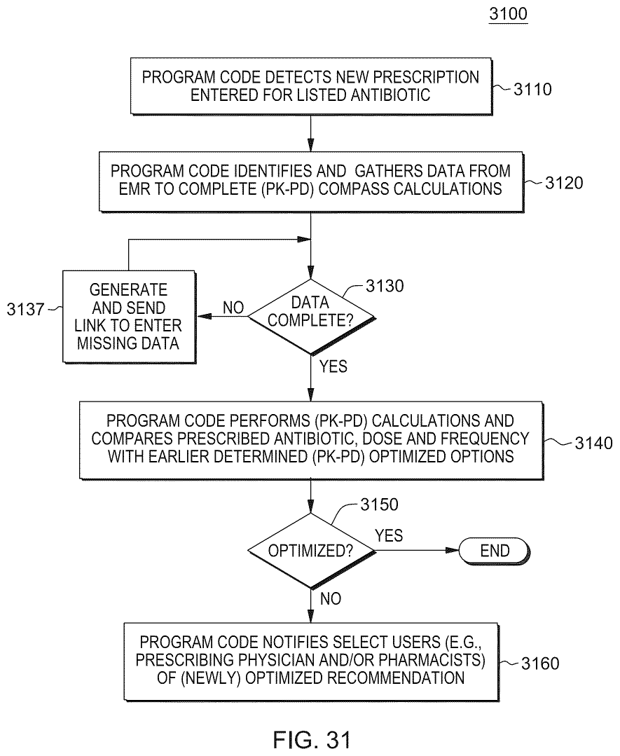

The present disclosure describes both the functionalities referred to as the PK-PD compass and the service-based implementation that can include aspects of the PK-PD compass. To that end, described herein are computing environments into which aspects of the present invention can be implemented, including the PK-PD compass as well as the trigger event reaction services, the workflow related to the PK-PD compass, a specific example of an interface that can be utilized to access the PK-PD compass, various workflows related to the implementation of the PK-PD compass and the service-based functionality implementation, and a general overview of the service-based implementation of aspects that include the PK-PD compass. To that end, FIGS. 1-30C focus on the PK-PD compass while starting with FIG. 31, the disclosure focusses on the service-based implementations where program code determines that trigger events have occurred within one or more EMR systems and determines and implements a response to the trigger events, if the program code determined that the events are material.

FIG. 1 is a computer system 100 configured to perform at least one aspect of an embodiment of the present invention. In the embodiment of FIG. 1, software 10 is executed by at least one processor on a computer, termed a base computer 12 in FIG. 1 for clarity. The terms software, program code, computer program code, code, computer program product, and executable instructions are used interchangeably throughout this application.

The software comprises code that is accessible to the processor and executable by at least one processor of the computer 12. The software can be stored on a memory on the physical computer 12, and/or in a memory and/or on removable media accessible to the computer 12 via a network connection, including but not limited to, a wireless and/or wireless network, utilizing a protocol known to one of skill in the art. The computer may also be configured to act as a web server, which may be capable of running the software and hosting and/or interacting with the database 14. Additionally, the computer can be one or more resources of a cloud computing system, executing the software performing the method described herein, which is accessible to a user as a service. In some of these embodiments of the present invention, any personally identifiable information can be stored locally or not utilized, to assuage any security concerns. However, by storing certain of the data in the cloud that does not cannot be used to personally identify patients, the data stored can be utilized by the program code for machine learning and to train the base model utilized to generate ranked options for users.

The base computer 12, as well as any other computer described in the present specification can includes personal computers, servers, smart phones, mobile devices, laptops, desktops, and/or any means of personal or corporate computing device capable of executing the software 10 or portions of the software 10 or communicating with a computer executing the software 10 over a wireless or hard-wired network.

In the embodiment of FIG. 1, the base computer is connected to a computer network 16, including but not limited to private and publicly accessible wired and wireless networks, and the Internet. In this embodiment, one or more computers, termed auxiliary computers 18a-18c are communicatively connected to the computer 12 via a computer network 16, including but not limited to, the Internet. The auxiliary computer 18a-18c receive data from the computer 12, via, for example, the web application server on the computer 12 and the auxiliary computers 18a-18c can render (for viewing) determinations regarding the probability of attaining a PK-PD target associated with efficacy for various antibiotics for the treatment of given patients based on data describing the given patient obtained at the base computer 12 and/or stored on the database 14 accessible to a processing resource on the base computer 12, including but not limited to, demographic information, and/or clinical laboratory data. The base computer can obtain data from the auxiliary computers 18a-18c, including but not limited to, the aforementioned descriptive data regarding the given patient for whom an antibiotic option is sought. As understood by one of skill in the art, the program code in various embodiments of the present invention can be stored on a memory resource and/or executed on one or more of the base computers 12 and/or the auxiliary computers 18a-18c.

The base computer 12 in the embodiment of FIG. 1 includes a database 14. Additional embodiments of the present invention utilize databases and other memory devices in different physical locations that are remotely accessible to the base computer 12 executing the software 10. In the embodiment of FIG. 1, the database 14 stores data including, but not limited to, population pharmacokinetic model-based equations and/or tabular outputs for a listing of potentially useful antibiotics that can be used to treat selected infectious diseases, information relating to various antibiotics and patients to whom they were offered by the present method, and/or the results of options returned by embodiments of the present invention.

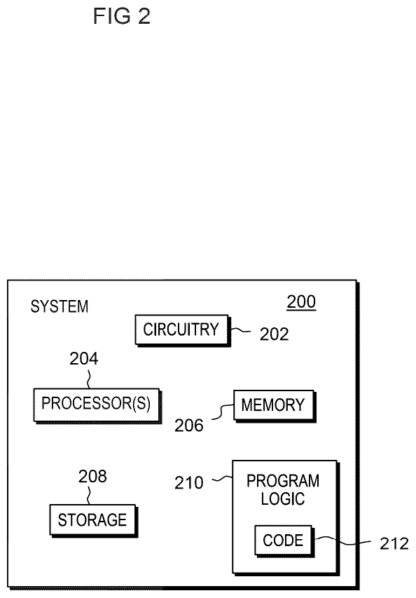

FIG. 2 illustrates a block diagram of a resource 200, like base computer 12 and/or auxiliary computers 18a-18c, in computer system 100, which is part of the technical architecture of certain embodiments of the technique. The resource 200 may include a circuitry 202 that may in certain embodiments include a microprocessor 204. The computer system 200 may also include a memory 206 (e.g., a volatile memory device), and storage 208. The storage 208 may include a non-volatile memory device (e.g., EEPROM, ROM, PROM, RAM, DRAM, SRAM, flash, firmware, programmable logic, etc.), magnetic disk drive, optical disk drive, tape drive, etc. The storage 208 may comprise an internal storage device, an attached storage device and/or a network accessible storage device. The system 200 may include a program logic 210 including code 212 that may be loaded into the memory 206 and executed by the microprocessor 204 or circuitry 202.

In certain embodiments, the program logic 210 including code 212 may be stored in the storage 208, or memory 206. In certain other embodiments, the program logic 210 may be implemented in the circuitry 202. Therefore, while FIG. 2 shows the program logic 210 separately from the other elements, the program logic 210 may be implemented in the memory 206 and/or the circuitry 202.

Using the processing resources of a resource 200 to execute software, computer-readable code or instructions, does not limit where this code can be stored. The terms program logic, code, and software are used interchangeably throughout this application.

As will be discussed herein, the implementation of certain aspects discussed herein as a service can involve a technical architecture that includes an EMR system and one or more interfaces to this system. The EMR and the additional systems which comprise the technical environment in which the service runs (e.g., as a background process) can each comprise one or more base computers 12. In fact, the program code of the service as well as the EMR system and the various interfaces to this system can be implemented in an enterprise system, including but not limited to, a cloud computing system.

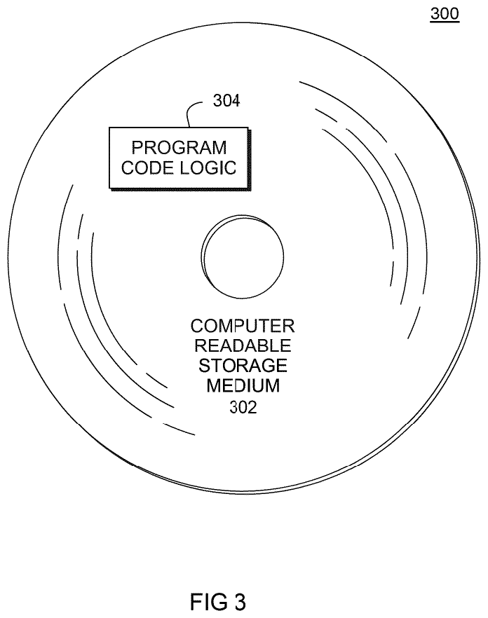

Referring to FIG. 3, in one example, a computer program product 300 includes, for instance, one or more non-transitory computer readable storage media 302 to store computer readable program code means or logic 304 thereon to provide and facilitate one or more aspects of the technique.

As will be appreciated by one skilled in the art, aspects of the technique may be embodied as a system, method or computer program product. Accordingly, aspects of the technique may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the technique may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable signal medium may include a propagated data signal with computer readable program code embodied therein, for example, in baseband or as part of a carrier wave. Such a propagated signal may take any of a variety of forms, including, but not limited to, electro-magnetic, optical or any suitable combination thereof. A computer readable signal medium may be any computer readable medium that is not a computer readable storage medium and that can communicate, propagate, or transport a program for use by or in connection with an instruction execution system, apparatus or device.

A computer readable storage medium may be, for example, but not limited to, an electronic, magnetic, optical, electromagnetic, infrared or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Program code embodied on a computer readable medium may be transmitted using an appropriate medium, including but not limited to wireless, wireline, optical fiber cable, RF, etc., or any suitable combination of the foregoing.

Computer program code for carrying out operations for aspects of the technique may be written in any combination of one or more programming languages, including an object-oriented programming language, such as Java, Smalltalk, C++ or the like, and conventional procedural programming languages, such as the “C” programming language, assembler or similar programming languages. The program code may execute entirely on the user's computer, partly on the user's computer, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the technique are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions, also referred to as computer program code, may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the technique. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In addition to the above, one or more aspects of the technique may be provided, offered, deployed, managed, serviced, etc. by a service provider who offers management of customer environments. For instance, the service provider can create, maintain, support, etc. computer code and/or a computer infrastructure that performs one or more aspects of the technique for one or more customers. In return, the service provider may receive payment from the customer under a subscription and/or fee agreement, as examples. Additionally, or alternatively, the service provider may receive payment from the sale of advertising content to one or more third parties.

In one aspect of the technique, an application may be deployed for performing one or more aspects of the technique. As one example, the deploying of an application comprises providing computer infrastructure operable to perform one or more aspects of the technique.

As a further aspect of the technique, a computing infrastructure may be deployed comprising integrating computer readable code into a computing system, in which the code in combination with the computing system is capable of performing one or more aspects of the technique. As a further aspect of the technique, the system can operate in a peer-to-peer mode where certain system resources, including but not limited to, one or more databases, is/are shared, but the program code executable by one or more processors is loaded locally on each computer (workstation).

As yet a further aspect of the technique, a process for integrating computing infrastructure comprising integrating computer readable code into a computer system may be provided. The computer system comprises a computer readable medium, in which the computer medium comprises one or more aspects of the technique. The code in combination with the computer system is capable of performing one or more aspects of the technique.

Further, other types of computing environments can benefit from one or more aspects of the technique. As an example, an environment may include an emulator (e.g., software or other emulation mechanisms), in which a particular architecture (including, for instance, instruction execution, architected functions, such as address translation, and architected registers) or a subset thereof is emulated (e.g., on a native computer system having a processor and memory). In such an environment, one or more emulation functions of the emulator can implement one or more aspects of the technique, even though a computer executing the emulator may have a different architecture than the capabilities being emulated. As one example, in emulation mode, the specific instruction or operation being emulated is decoded, and an appropriate emulation function is built to implement the individual instruction or operation.

In an emulation environment, a host computer includes, for instance, a memory to store instructions and data; an instruction fetch unit to fetch instructions from memory and to optionally, provide local buffering for the fetched instruction; an instruction decode unit to receive the fetched instructions and to determine the type of instructions that have been fetched; and an instruction execution unit to execute the instructions. Execution may include loading data into a register from memory; storing data back to memory from a register; or performing some type of arithmetic or logical operation, as determined by the decode unit. In one example, each unit is implemented in software. For instance, the operations being performed by the units are implemented as one or more subroutines within emulator software.

Further, a data processing system suitable for storing and/or executing program code is usable that includes at least one processor coupled directly or indirectly to memory elements through a system bus. The memory elements include, for instance, local memory employed during actual execution of the program code, bulk storage, and cache memory which provide temporary storage of at least some program code to reduce the number of times code must be retrieved from bulk storage during execution.

Input/Output or I/O devices (including, but not limited to, keyboards, displays, pointing devices, DASD, tape, CDs, DVDs, thumb drives and other memory media, etc.) can be coupled to the system either directly or through intervening I/O controllers. Network adapters may also be coupled to the system to enable the data processing system to become coupled to other data processing systems or remote printers or storage devices through intervening private or public networks. Modems, cable modems, and Ethernet cards are just a few of the available types of network adapters.

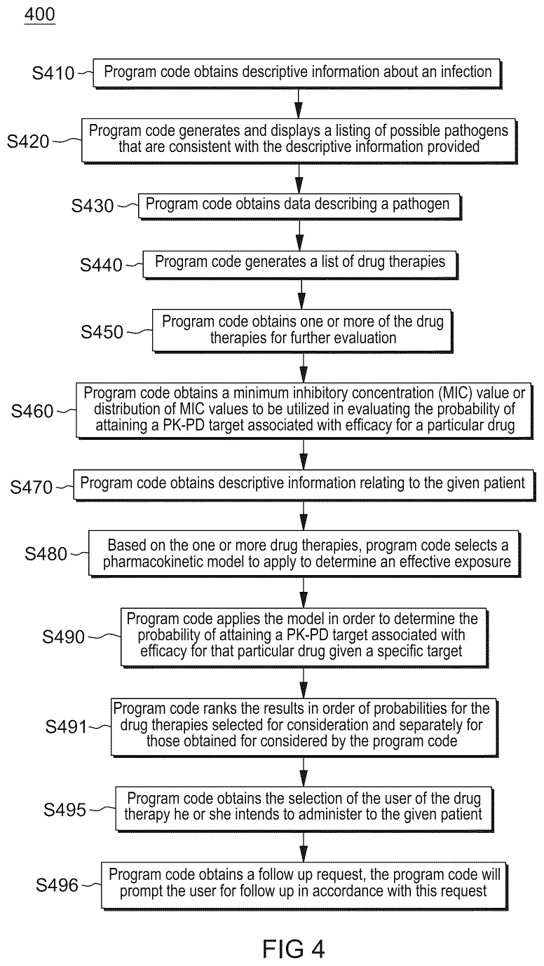

FIG. 4 depicts a workflow 400 of aspects of an embodiment of the present technique. To estimate the anti-infective drug exposure for a given patient, program code executed by at least one processor on a computer resource, such as resource 200, obtains descriptive information about an infection (S410), including but not limited to, where the infection is located, and/or where the infection was acquired.

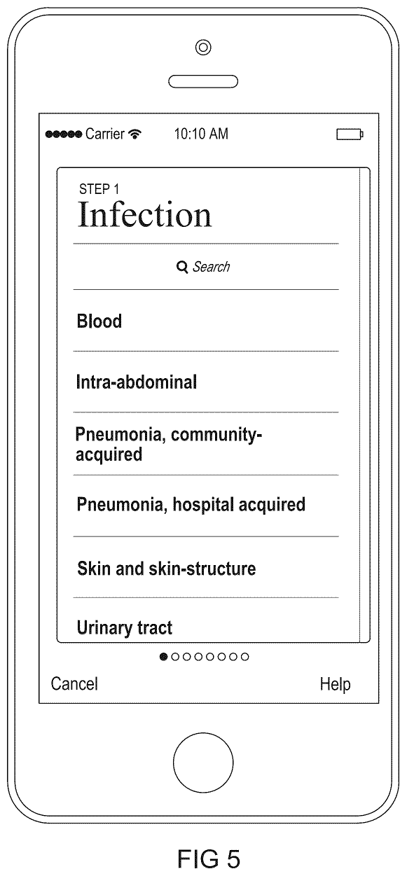

FIG. 5 depicts an example of a graphical user interface 510 on a mobile device 500, which is a computer resource that can be an aspect of embodiments of the present invention. The screen depicted in FIG. 5 is termed the “Infection” screen where a user, for example, a clinician, can select descriptive terms to assist the program code in executing on a processor of this computer resource in obtaining further characteristics about the infection.

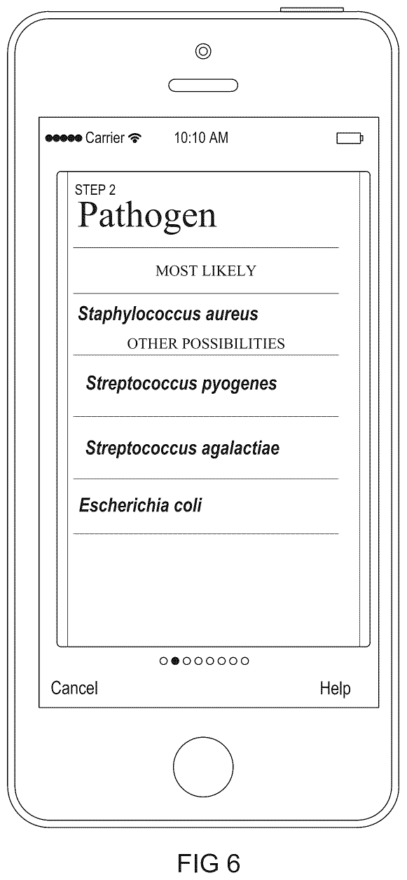

Returning to FIG. 4, upon obtaining descriptive information about the infection, the program code generates and displays a listing of possible pathogens that are consistent with the descriptive information provided (S420). As seen to FIG. 6, which depicts a GUI with an exemplary screen listing possible pathogens, the listing generated by the program code can include an indication of what is the most likely and/or possible pathogen, based on the description. In an embodiment of the present invention, the program code executed by the processor can make this determination by accessing data on a storage medium that is located either local to the computer resource or accessible via a communications connection.

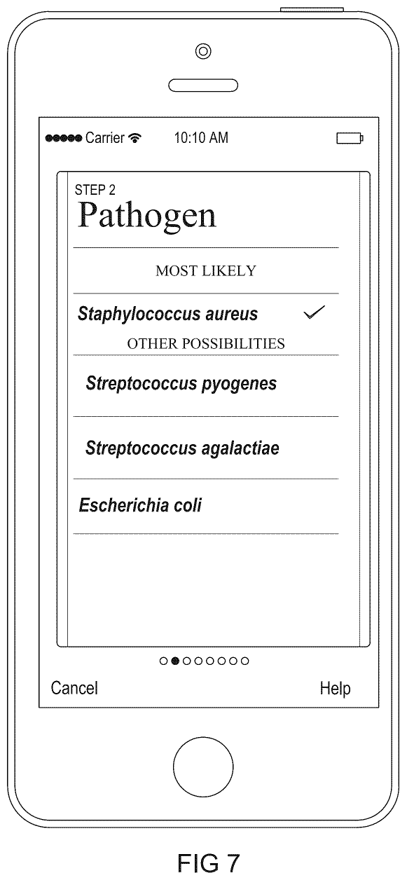

Returning to FIG. 4, the program code executed by a processor of the computer resource obtains data describing a pathogen (S430). As seen in FIG. 7, a user of this embodiment of the present invention, the program code can obtain the pathogen when the user makes a selection in the GUI that is displayed by the program code listing possible pathogens.

Responsive to receiving the data describing a pathogen, the program code executed by a processor generates a list of drug therapies (S440), including but not limited to, antibiotics, which are options for treating the pathogen. The list generated by the program code executed by a processor can include a single result or a group of results, based upon the information obtained.

In an embodiment of the present invention, data related to drug therapies that may comprise the list created by the program code can be stored on a memory resource that is integral to the computer resource and/or accessible to the computer resource via a communications connection.

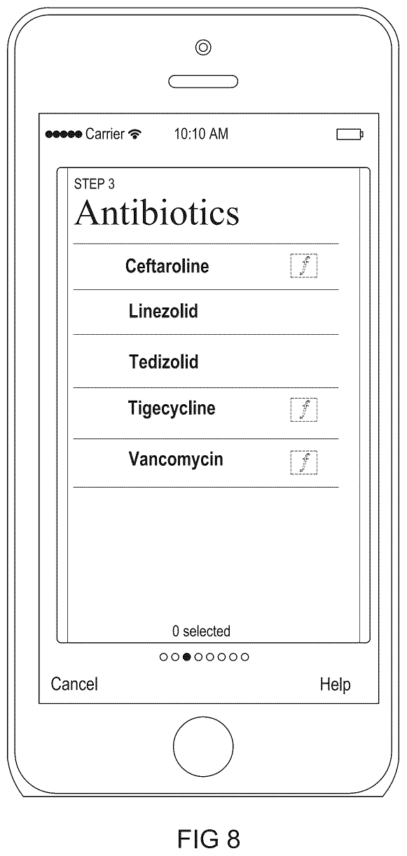

FIG. 8 is an example of a list of drug therapies generated by program code in an embodiment of the present invention. In the example of FIG. 8, a list of antibiotics was generated by the program code and displayed to the user. From this list of drug therapies related to the identified pathogen and/or infection, in an embodiment of the present invention, the user, such as a clinician, can select from the list one or more drug therapies for further evaluation to receive the probability of attaining the PK-PD target associated with efficacy for the drug in treating a given patient. In an embodiment of the present invention, the drug therapies that are listed to a user by the program code comprise drug therapies that have known success in treating the pathogen obtained by the program code, e.g., identified by the user through an input into the computer resource.

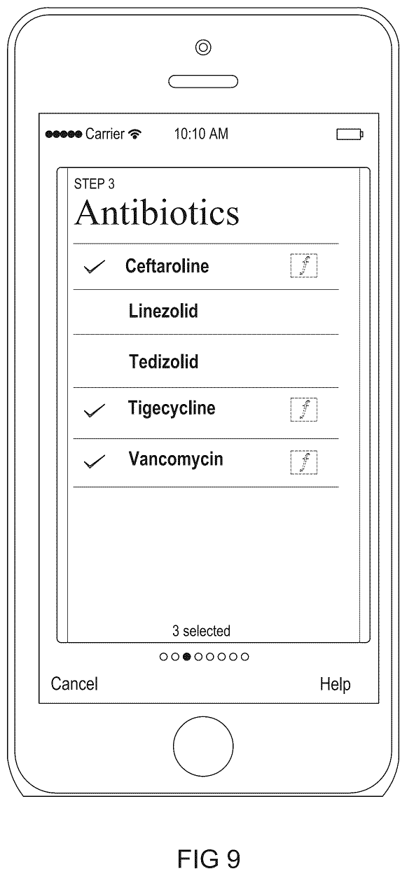

Returning to FIG. 4, the program code obtains one or more of the drug therapies for further evaluation by the program code (S450). As aforementioned, the program code can receive the one or more drug therapies for further evaluation based upon a selection made by the user using an input device, such as a touch screen. FIG. 9 is an example of a screen of a GUI, in an embodiment of the present invention, where a user has selected drug therapies, in this example, antibiotics, for further analysis by the program code. In an embodiment of the present invention, in addition to the program code listing drug therapies from which a user can select, a user can also enter one or more drug therapies for evaluation. The program code may retain the entered drug therapies and save the drug therapies and their relationship in treating a given pathogen, on a memory resource, for future use, including but not limited to one or more remote memory resource(s). In an embodiment of the present invention, the program code obtains a listing of potentially useful antibiotics that can be used to treat selected infectious diseases indicated by the infections and/or pathogens experienced by the given patient from a memory resource.

In a further embodiment of the present invention, the program code evaluates all the drug therapies provided rather than enable a user, or an automatic process, to limit the number of therapies further evaluated.

Returning to FIG. 4, the program code also obtains a minimum inhibitory concentration (MIC) value or distribution of MIC values to be utilized in evaluating the probability of attaining a PK-PD target associated with efficacy for a particular drug (S460). The user can select the type of MIC distribution that the program code will apply The MIC is the lowest concentration of an antimicrobial that will inhibit the visible growth of a microorganism after overnight incubation. Because the MIC value relates to an in vitro measurement of the efficacy of a drug, the efficacy of the drug therapy, as related to a given patient, is not immediately apparent without utilizing additional parameters and applying a relevant pharmacokinetic model, which is discussed later.

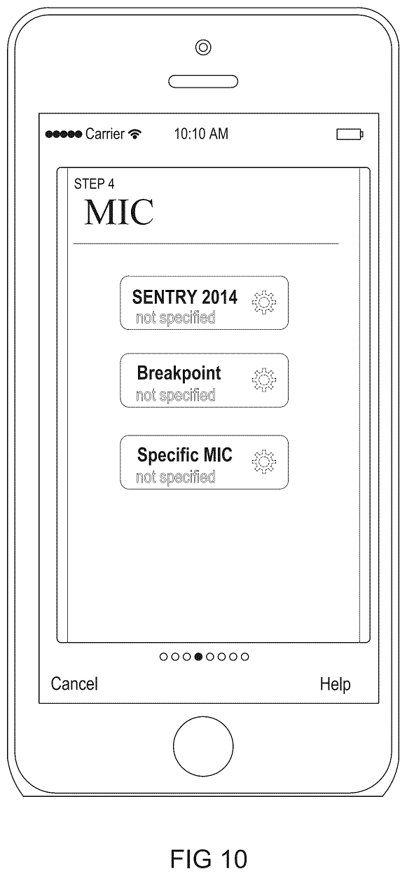

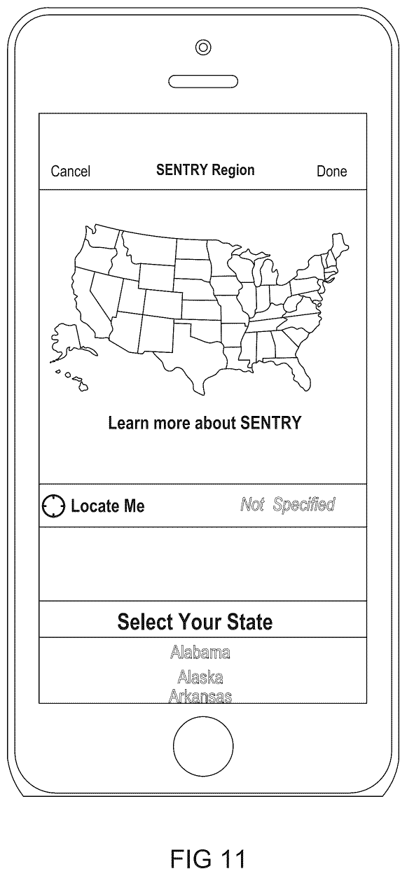

Depending upon the type of drug therapies being contemplated, the user may select a MIC distribution rather than a fixed MIC value. FIG. 10 is an example of a GUI utilized in an embodiment of the present invention to display to a user select choices of MIC values. As susceptibility patterns and hence, likely MIC values may be influenced by the geographical location of a given patient, should a user select a distribution of MIC values based on surveillance data, including but not limited to, the SENTRY repository of data, such as SENTRY 2014), the user can be prompted to enter the location of the patient, as seen in FIG. 11.

In an embodiment of the present invention, the computer resource can include a GPS that the program code utilizes to find the location of the user and therefore, apply the relevant MIC distribution. As seen in FIG. 11, the program code displays a “Locate me” function. In this example, when a user selects this location function, the program code will request location information from the GPS and receive this information, which it will use to generate a user location, responsive to the request.

Returning to FIG. 4, as discussed earlier, demographic information related to the patient is also utilized by the program code determining the probability of attaining PK-PD targets associated with efficacy for known drug therapies. Thus, the program code obtains descriptive information relating to the given patient (S470). The descriptive information includes, but is not limited to, the weight of the patient and the creatinine clearance of the given patient.

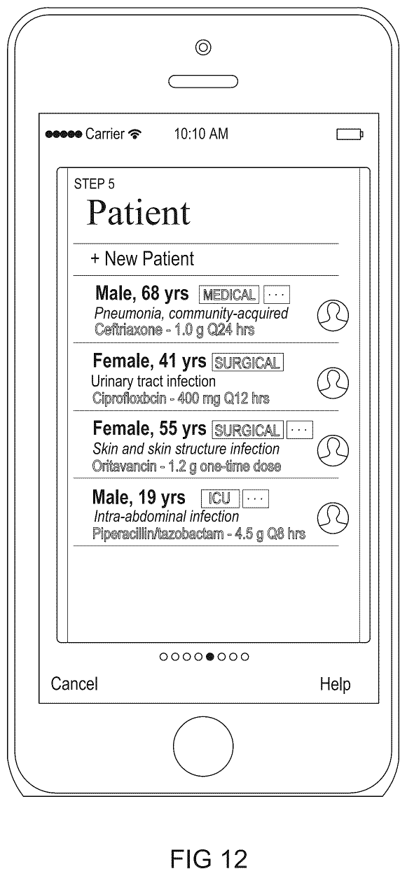

In an embodiment of the present invention, the program code displays a list of descriptive information relating to existing patients, enabling the user to select a patient from this listing. The existing patient records may be retained on an accessible memory resource, such as a database. An example of a GUI where the program code renders a list of existing patients is displayed as FIG. 12.

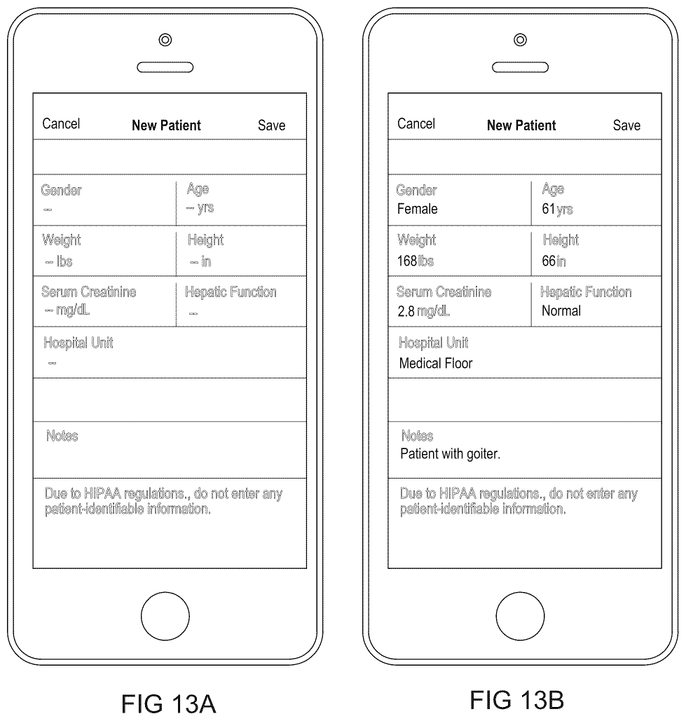

In an embodiment of the present invention, program code executed by a processor can obtain user information from user entry. For example, a user can enter patient information related to a new patient. This option is also visible in FIG. 12 and if selected, in an embodiment of the present invention, the program code produces a GUI where the user can enter new patient information, as seen in FIGS. 13A and 13B. As aforementioned, among the parameters requested in the GUI and therefore, received by the program code, are the patient's weight and serum creatinine. As seen in FIGS. 13A and 13B, in compliance with HIPAA regulations, the GUI where a user can enter patient information can be configured to warn a user not to enter any patient-identifiable information. Additionally, in an embodiment of the present invention, the program code does not obtain and/or retain private information in violation of HIPAA.

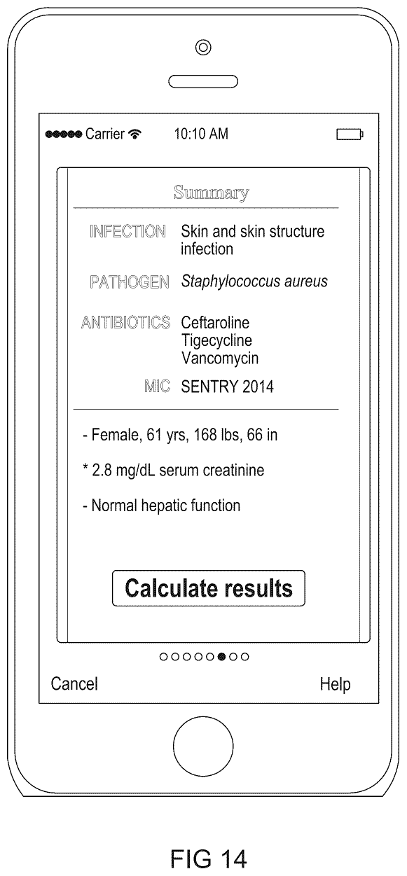

Once the program code has obtained the drug therapy being considered, including descriptive factors that may include, but are not limited to, the dosage, duration of infusion, and/or dosing interval, the MIC or the MIC distribution, and the aforementioned patient characteristics, the program code determines the probability of attaining the PK-PD target associated with efficacy for the selected drug therapy and/or therapies. In an embodiment of the present invention, the program code executed by a processor displays a summary screen to a user that includes the data obtained that the program code will utilize to determine PK-PD target attainment. FIG. 14 is an example of a summary screen.

Referring to FIG. 14, the summary screen lists the infection, the pathogen, the selected drug therapies, which, in this example, are antibiotics, and the selection made for MIC. The summary screen also lists descriptive information about the patient, in this example, the gender, age, weight, height, serum creatinine, and category of hepatic function. The program code will vary the parameters utilized in determining the probability of attaining a PK-PD target associated with efficacy for each therapy, based upon that therapy. For example, while the height of the patient may assist in a determination for a given drug therapy, that parameter may not be used by the program code in determining the probability of attaining a PK-PD target associated with efficacy for a different drug. Thus, returning to FIG. 4, based upon each drug therapy selected, the program code selects a pharmacokinetic model to apply to determine an exposure and the probability of attaining a PK-PD target associated with efficacy for that drug therapy, which in an embodiment of the present invention, is expressed as a percent probability (S480). The program code utilizes at least one pharmacokinetic model in determining the probability of attaining a PK-PD target associated with efficacy for a given drug therapy for a given patient. Thus, upon selecting a pharmacokinetic model, the program code applies the model to determine the probability of attaining a PK-PD target associated with efficacy for that particular drug given a specific target (S490).

The pharmacokinetic models associated with different drug therapies use mathematical representations of parts of the body to describe the time-course of drug concentrations in the body. To describe the parts of the body affecting the time-course of drug concentrations, the body of the patient can be understood as containing compartments. The models account for n number of compartments. Some models utilize three compartments. Taking the drug therapy, meropenem as an example, its pharmacokinetics can be described using two compartments. The two compartments represent blood and tissue. This two-compartment type of pharmacokinetic model is applied during and after infusion.

In a two-compartment pharmacokinetic model discussed later in this document, Vc stands for “volume of the central compartment” which is usually blood. Thus, when a drug is infused (Ko), it will be input into this compartment. The second compartment, Vp, stands for “peripheral compartment” which approximates the tissue. The transfer rate of drug between these two compartments is called “distributional clearance” (CLd). In the central compartment, drug will be eliminated (by routes such as renal excretion or metabolism) and this is considered an output and is termed “total clearance” (CLt). These parameters can be calculated if equations are known for a given drug therapy, and, as aforementioned, for most drug therapies, the patient weight, and creatinine clearance for a given patient are also known.

Returning to FIG. 14, when utilizing the GUI of an embodiment of the present invention, the user can visually verify that the information on the summary screen is correct and submit this information to the program code for determination of the probability of attaining a PK-PD target associated with efficacy for each selected drug therapy, as discussed in reference to FIG. 4.

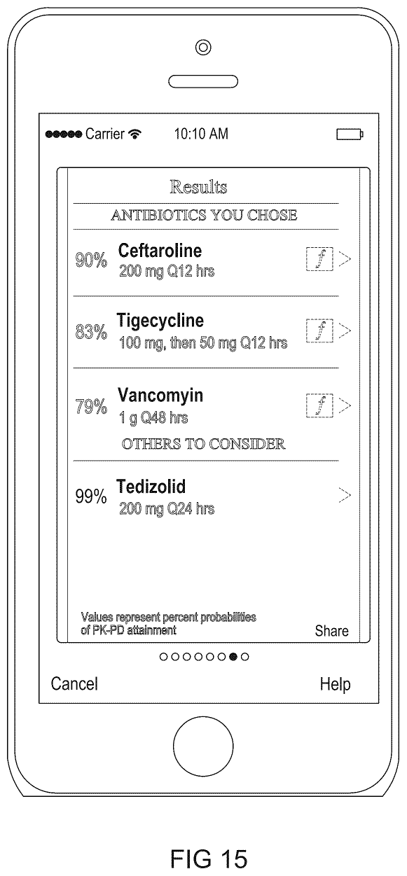

In an embodiment of the present invention, once the program code has determined a probability of attaining a PK-PD target associated with efficacy for each selected drug therapy and/or drug therapies that were not selected by the user, the program code ranks the results in order of probabilities for the drug therapies selected for consideration and separately for those obtained for considered by the program code (S491). FIG. 15 is an example of a screen of a GUI utilized in an embodiment of the present invention to display the determined probabilities of PK-PD target attainment and rank the drug therapies by these probabilities.

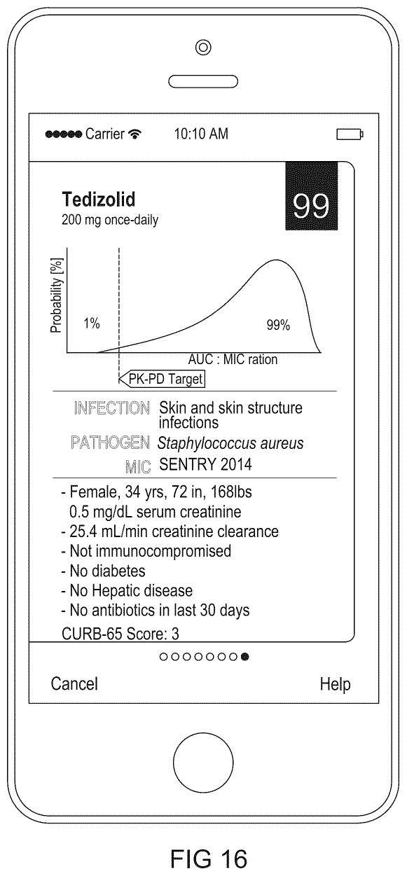

After providing a user with the probability of attaining a PK-PD target associated with efficacy for drug therapies considered, in an embodiment of the present invention, the program code can obtain the selection of the user of the drug therapy he or she intends to administer to the given patient (S495). In an embodiment of the present invention, the program code retains the selection on a memory device accessible to the processor. In an embodiment of the present invention, the program code can generate a GUI that displays individual results for the probability of attaining a PK-PD target associated with efficacy for various drug therapies from a listing screen, such as FIG. 15. FIG. 16 is an example of this type of detail screen, in this case, for the drug therapy Tedizolid. The probability of attaining the PK-PD target associated with efficacy for the drug therapy is presented in the context of the infection and/or pathogen obtained by the program code upon entry of information by a user.

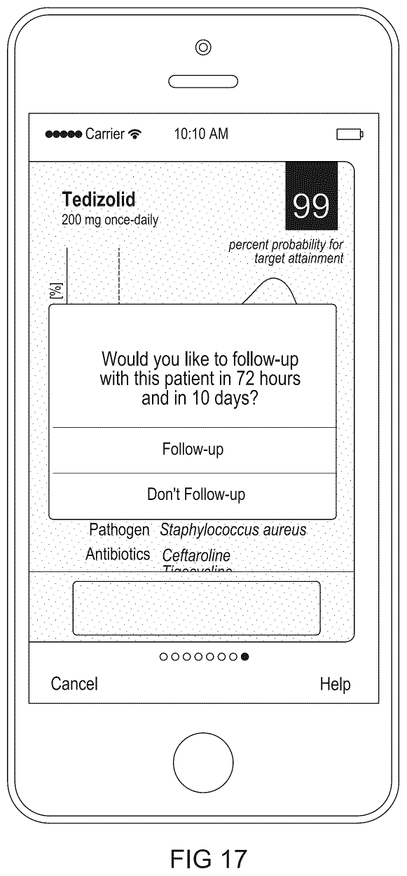

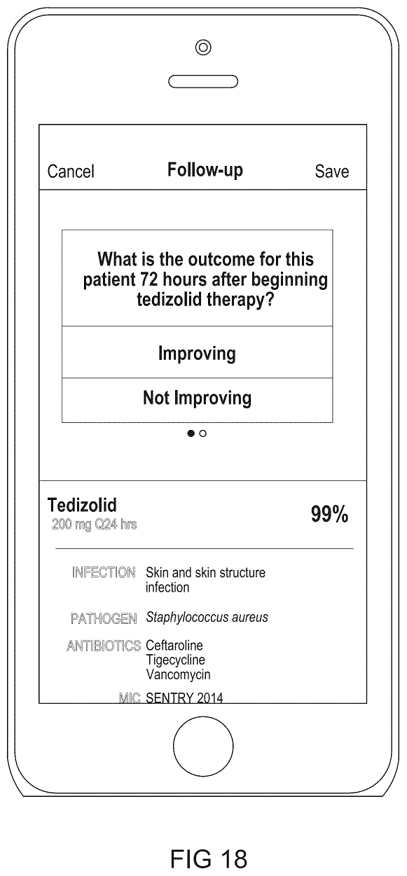

In an embodiment of the present invention, the user can track the actual efficacy of the drug therapy selected, for example, to compare and contrast the expected outcome with the actual outcome. In FIG. 17, once the user has selected the drug therapy from the drug therapies returned as options, the user can select whether he or she would like to be prompted to follow up with the patient. Should the user opt to follow up, in an embodiment of the present invention, the program code will display a reminder to the user to follow up regarding a given patient. FIG. 18 is an example of a possible display for this follow up activity and additionally may collect information regarding efficacy, including but not limited to, requesting that a user enter information and/or importing information from an external data repository. In an embodiment of the present invention, the reminder generated by the program code can be audible and/or visual.

Returning to FIG. 4, in an embodiment of the present invention where the program code obtains a follow up request, the program code will prompt the user for follow up in accordance with this request (S496).

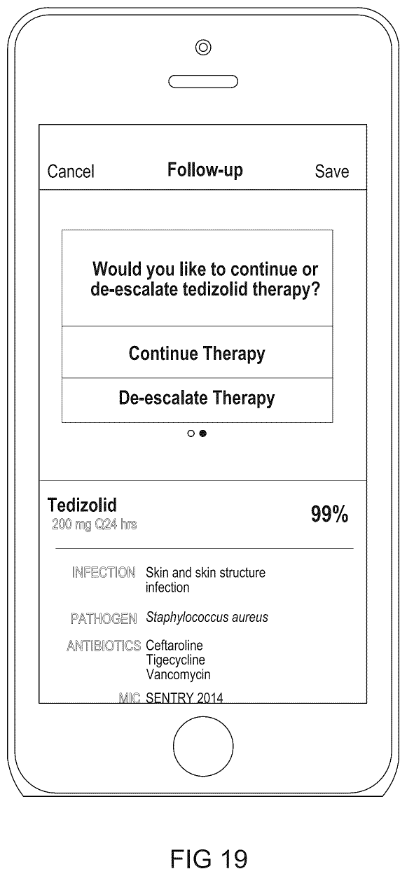

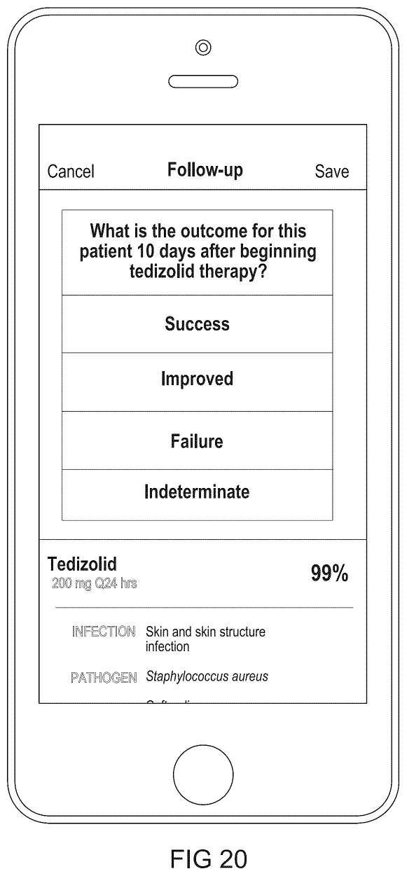

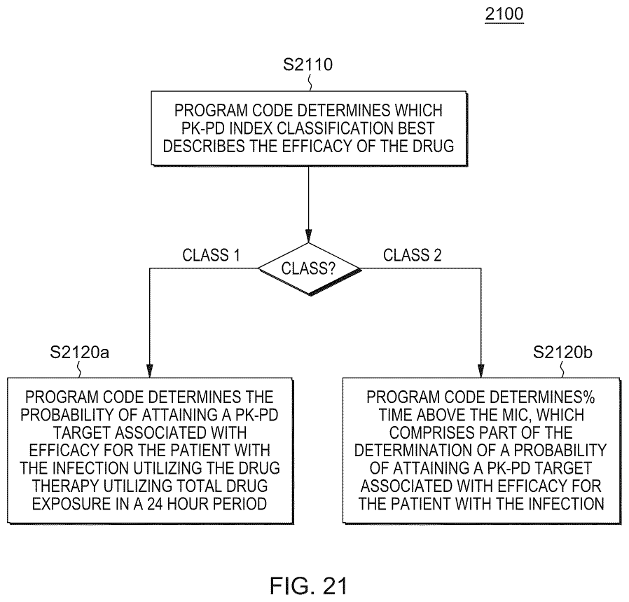

FIG. 19 is an example of a Follow-up screen that can be utilized to note the efficacy of the drug therapy when administered to the given patient. FIGS. 19-20 represent different types of questions/data that can be asked/collected by the program code to track the success of the selected treatment.

As aforementioned when discussing FIG. 4, the program code selects a pharmacokinetic model to apply to determine the probability of attaining a PK-PD target associated with efficacy for a given drug therapy. The following sections provide examples of the models that can be applied in various embodiments of the present invention and how these models can be applied by the program code. FIG. 21 detail various determinations made by the program code in selecting and applying a pharmacokinetic model to a given drug therapy.

In an aspect of the present invention, to select the pharmacokinetic model, the program code first determines which PK-PD index classification best describes the efficacy of the drug. While there are more than two possible categories for this classification, as one example, FIG. 21 provides an example based on only two categories (S2110). In the first class, the probability of attaining the PK-PD target associated with efficacy for a drug therapy is determined at least in part, based upon the total drug exposure in a 24-hour period, wherein the AUC:MIC ratio is at least part of the determination (S2120a). In the second class, the % time above MIC is calculated and comprises at least a portion of the determination (S2120b). Despite the categorization of drugs into these two distinct classes by the program code, the model applied by the program code will differ between drugs. Depending upon a drug therapy selected, the program code may apply a customized model, including varying the parameters requested from a user, to ultimately determine the probabilities displayed. However, in embodiments of the present invention, the class to which the drug belongs directs the type of model utilized by the program code.

An example of one drug therapy that would be classified in the first category is ciprofloxacin. As aforementioned, the program code selects and applies the models based upon the drug therapy itself. However, the patient characteristics and MIC obtained by the program code affect the resulting prediction of PK-PD target attainment.

In the equations below, an estimated probability of attaining a PK-PD target associated with efficacy for ciprofloxacin is determined based upon parameters related to ciprofloxacin and obtained by the program code in the manner described in FIG. 4. The parameters obtained by the program include, but are not limited to, the drug therapy and dosage, which in the example below, the drug therapy is ciprofloxacin which is given every 8 hours intravenously. The patient characteristics are:
1) Creatinine clearance (CLcr): 63 mL/min; and
2) Weight (WT);

The MIC in the example below is 1 mg/L

Utilizing parameters specific to ciprofloxacin, the program code determines the area under the curve over 24 hours (AUC₂₄). Equation 1 is an example of an Equation that the program code can utilize to make this determination. In the Equation 2, below, the AUC₂₄ is used to find the total clearance (CLt).

$${AUC}_{24} = \frac{Daily Dose}{C L t} \qquad ( Equation l )$$

$$CLt - ( 0.00 \; 145 \times CLcr + 0.1 \; 67 ) \times WT \qquad ( Equation 2 )$$

By applying the parameters discussed, the following calculations can be made:

$$Daily dose = 400 mg / 8 h r \times 24 hr = 1200 mg$$

$$CLt = ( 0.00145 \times 63 + 0.167 ) \times 70 = 18.1 L / hr$$

$$AU {C}_{24} = 1200 / 18.1 = 66.4 mg / L \times hr$$

$$AUC : MIC ratio = \frac{A U {C}_{24}}{MIC} = \frac{66.4}{1} = 66.4$$

Once the AUC:MIC ratio is calculated, it is compared to the threshold for AUC:MIC ratio associated with efficacy (i.e., the PK-PD target). If it is above the PK-PD target, a patient is more likely to have a successful response to therapy; if it is below, the patient is less likely. A point estimate for probability of PK-PD target attainment will be determined as a function of the AUC:MIC ratio. The variability about this estimate is also determined by the program code. Thus, by obtaining parameters from a user and/or a memory resource, determining the relevant model, applying the model and using simulation, and returning a result to a user.

Returning to FIG. 21, if the drug therapy being evaluated by the program code is in the second class, the program code calculates the % time above the MIC, which comprises at least a portion of the determination (S2120b). As discussed earlier, in embodiments of the present invention, to predict the probability of attaining the PK-PD target associated with efficacy for a given drug therapy, the pharmacokinetic model applied depends upon the actual drug, so the program code determines what model to apply based upon the drug therapy. Meropenem is an example of a drug therapy that is a member of this class and is used as an example in explaining an example of a pharmacokinetic model applied to members of this PK-PD classification.

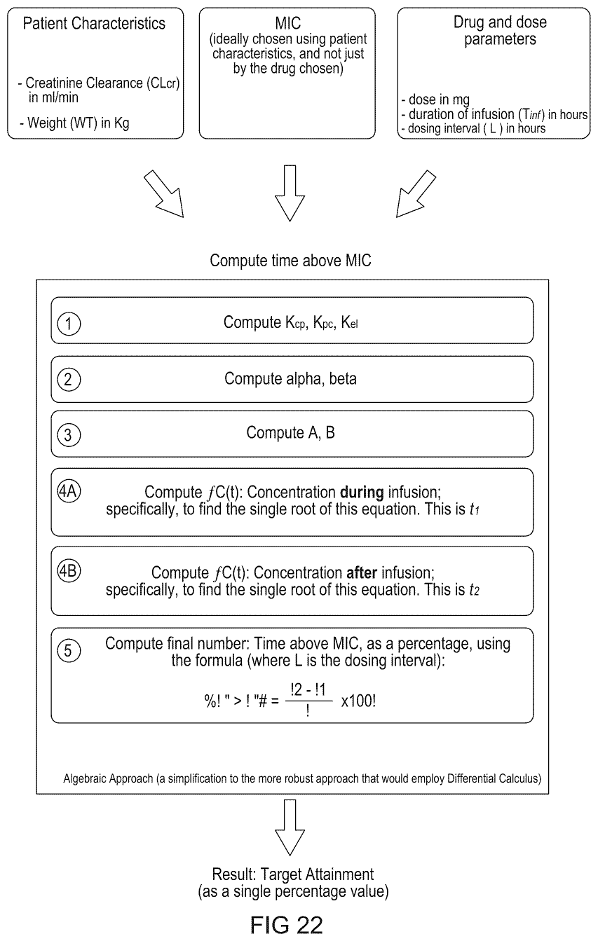

FIG. 22 is an example of the application of a pharmacokinetic model to determine the efficacy of the drug therapy for a given patient. Referring to FIG. 22, as explained earlier, the program code obtains patient characteristics, a MIC, and the drug therapy and dose parameters. The program code uses these parameters to compute the % time above MIC. To make this determination, the program code computes the Kcp, Kpc, and Kel values, the Alpha and Beta, the A and B and then, and uses these values to find the concentration during infusion, the concentration after infusion, and then applies these values to calculate a final efficacy percentage for the drug for the given patient. The derivation of these individual values is discussed below. The variables utilized in the present example are defined as follows: Kcp is the rate constant for flow from central to peripheral; Kpc is rate constant for flow from peripheral to central; Alpha is the rate constant for the first phase of drug elimination; Beta is the rate constant for the second phase of drug elimination; A is the concentration in the alpha phase at time 0; and B is the concentration in the beta phase at time 0.

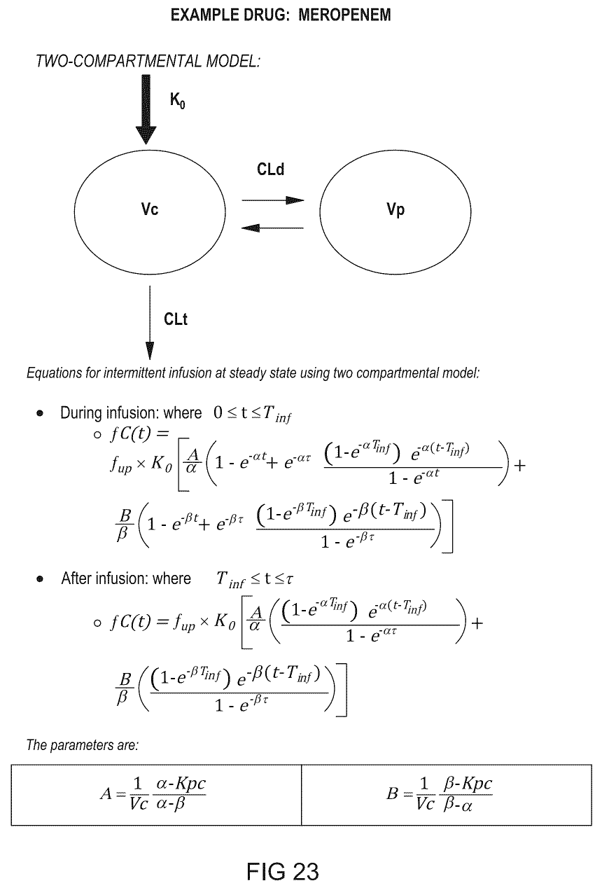

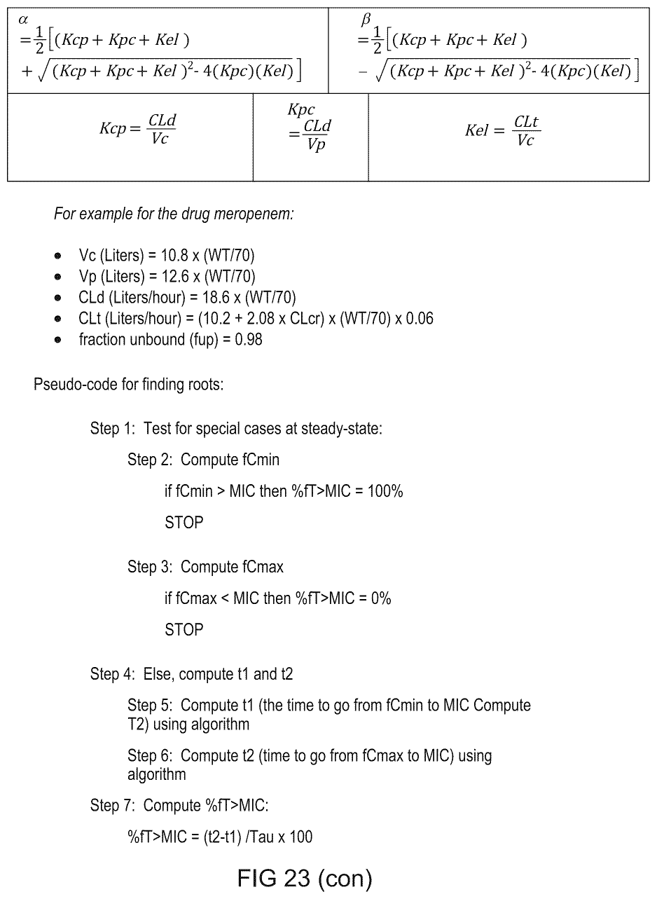

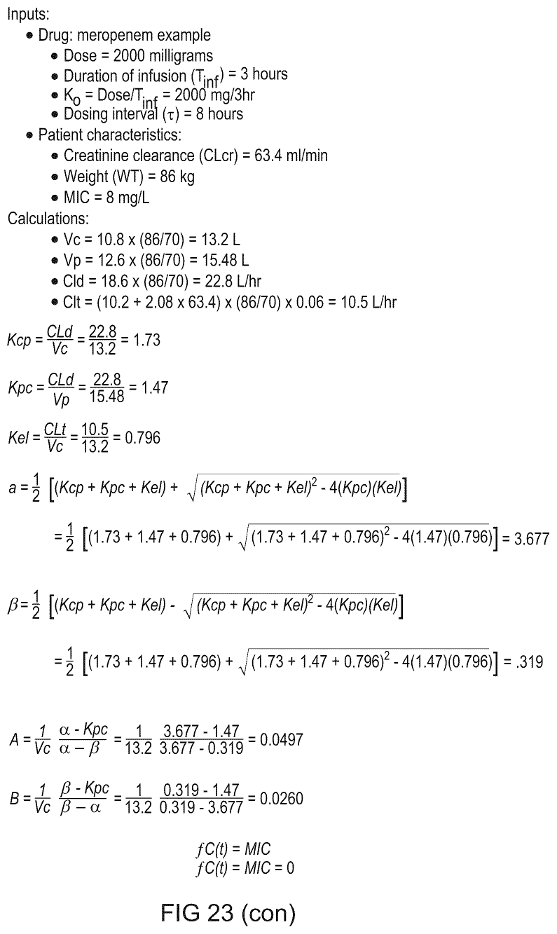

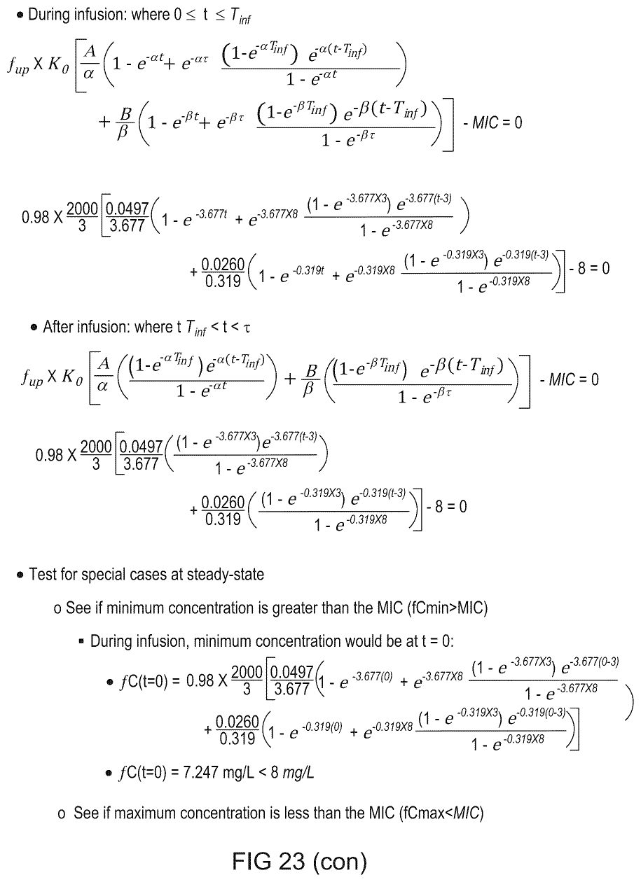

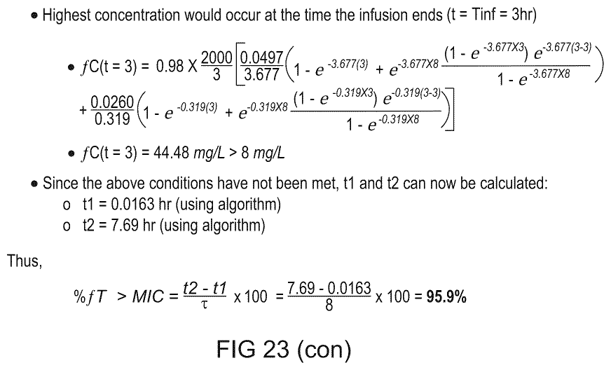

FIG. 23 shows an example of a two-compartment model that is utilized when evaluating meropenem. In the figure, the compartment with Vc stands for “volume of the central compartment” which is usually blood. Thus, when meropenem is infused (arrow with Ko), it will be inputted into this compartment. The second compartment, Vp, “peripheral compartment,” is for tissue. The transfer rate of drug between these two compartments is called “distributional clearance” (CLd). In the central compartment, drug will be eliminated (by routes such as renal excretion or metabolism) and this is considered an output (arrow going out to nowhere) and is termed “total clearance” (CLt).

Using a steady state model and the two-compartment model for and the drug meropenem, the Equation 3 and Equation 4 can be applied.

$$f C ( t ) = {f}_{u p} \times {K}_{0} [ \frac{A}{α} ( 1 - {e}^{- α t} + {e}^{- ατ} \frac{( 1 - {e}^{- α {T}_{inf}} ) {e}^{- α ( t - {T}_{inf} )}}{1 - {e}^{- a τ}} ) + \frac{B}{β} ( 1 - {e}^{- β t} + {e}^{- β τ} \frac{( 1 - {e}^{- β {T}_{inf}} ) {e}^{- β ( t - {T}_{inf} )}}{1 - {e}^{- β τ}} ) ] \qquad Equation 3$$

$$fC ( t ) = {f}_{u p} \times {K}_{0} [ \frac{A}{α} ( \frac{( 1 - {e}^{- {αT}_{inf}} ) {e}^{- α ( t - {T}_{inf} )}}{1 - {e}^{- ατ}} ) + \frac{B}{β} ( \frac{( 1 - {e}^{- β {T}_{inf}} ) {e}^{- β ( t - {T}_{inf} )}}{1 - {e}^{- β τ}} ) ] \qquad Equation 4$$

Table 1 below includes the parameters utilized by the above equations.

TABLE 1

| | |
|---|---|
| $A = \frac{1}{V c} \frac{α - K p c}{α - β}$ | $B = \frac{1}{V c} \frac{β - K p c}{β - α}$ |
| $α = \frac{1}{2} [ ( Kcp + Kpc + Kel ) + \sqrt{{( Kcp + Kpc + Kel )}^{2} - 4 ( kpc ) ( Kel )} ]$ | $β = \frac{1}{2} [ ( K c p + K p c + Kel ) - \sqrt{{( K c p + K p c + K e l )}^{2} - 4 ( K p c ) ( K e l )} ]$ |

| | | |
|---|---|---|
| $K c p = \frac{C L d}{V c}$ | $Kpc = \frac{C L d}{V p}$ | $Kel = \frac{C L t}{V c}$ |

Below are values that can be utilized in the present invention for meropenem. In an embodiment of the present invention, the values can be retained on a memory resource and identified and utilized by the program code upon the program code categorizing the drug by the PK-PD index and identifying the appropriate model.

For meropenem:

$$Vc ( Liters ) = 1 0.8 \times ( WT / 70 )$$

$$Vp ( Liters ) = 12.6 \times ( WT / 70 )$$

$$CLd ( Liters / hour ) = 18.6 \times ( WT / 70 )$$

$$CLt ( Liters / hour ) = ( 10.2 + 2.08 \times CLcr ) \times ( WT / 70 ) \times 0.06$$

$$fraction unbound ( fup ) = 0.98$$

The variables utilized in the present example are defined as follows: Kcp is the rate constant for flow from central to peripheral; Kpc is the rate constant for flow from peripheral to central; Alpha is the rate constant for the first phase of drug elimination; Beta is the rate constant for the second phase of drug elimination; A is the concentration in the alpha phase at time 0; and B is the concentration in the beta phase at time 0.

An embodiment of the present invention can obtain the following drug and dose information: Dose=2000 milligrams; Duration of infusion (T_{inf})=3 hours; K₀=Dose T_{inf}=2000 mg 3 hr; Dosing interval (r)=8 hours. This embodiment can also obtain the following patient characteristics: Creatinine clearance (CLcr)=63.4 mL/min; Weight (WI)=86 kg. The present invention also obtains the following MIC: MIC=8 mg/L. Utilizing these values, the program code can determine Kcp, Kpc, and Kel values, the Alpha and Beta, the A and B and then, and uses these values to find the concentration during infusion, the concentration after infusion, and then applies these values to calculate the probability of attaining the PK-PD target associated with efficacy for the drug for the given patient. In this example, the program code returns the value of 99% for the probability of attaining the PK-PD target associated with efficacy for this drug with these parameters.

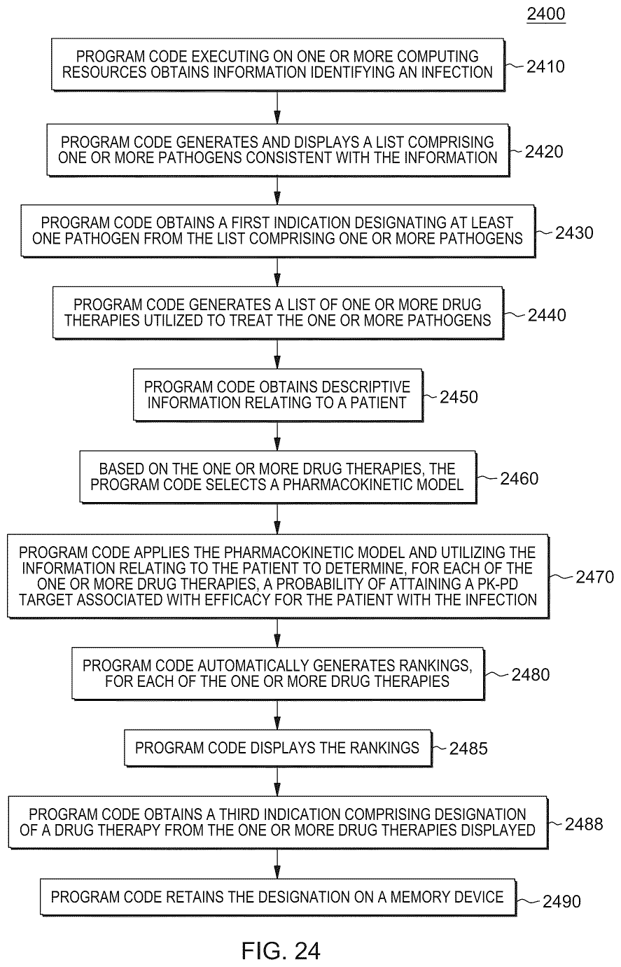

FIG. 24 is a workflow 2400 that illustrates certain aspects of some embodiments of the present invention utilized for determining a probability of attaining a PK-PD target associated with efficacy for a patient. In embodiment of the present invention, program code executing on one or more computing resources (including but not limited to one or more resources of a cloud computing environment), obtains information identifying an infection (2410). Based on the information, the program code generates and displays a list comprising one or more pathogens consistent with the information (2420). The program code obtains a first indication designating at least one pathogen from the list comprising one or more pathogens (2430). Based on at the obtaining the list, the program code generates a list of one or more drug therapies utilized to treat the one or more pathogens (2440). The program code obtains descriptive information relating to a patient (2450). The descriptive information can include, but is not limited to, an infection acquired by the patient, a pathogen isolated from the patient, a creatinine clearance of the patient, a weight of the patient, and a height of the patient.

In some embodiments of the present invention, the program code communicates with the patient and/or presents the patient (user) with an interface upon which to provide indications through a personal computing device that is an Internet of Things (IoT) device. This, the IoT device could passively and/or actively collect a certain portion of the descriptive information from the user. As understood by one of skill in the art, the Internet of Things (IoT) is a system of interrelated computing devices, mechanical and digital machines, objects, animals and/or people that are provided with unique identifiers and the ability to transfer data over a network, without requiring human-to-human or human-to-computer interaction. These communications are enabled by smart sensors, which include, but are not limited to, both active and passive radio-frequency identification (RFID) tags, which utilize electromagnetic fields to identify automatically and to track tags attached to objects and/or associated with objects and people. Smart sensors, such as RFID tags, can track environmental factors related to an object, including but not limited to, temperature and humidity. The smart sensors can be utilized to measure temperature, humidity, vibrations, motion, light, pressure and/or altitude. IoT devices also include individual activity and fitness trackers, which include (wearable) devices or applications that include smart sensors for monitoring and tracking fitness-related metrics such as distance walked or run, calorie consumption, and in some cases heartbeat and quality of sleep and include smartwatches that are synced to a computer or smartphone for long-term data tracking. Because the smart sensors in IoT devices carry unique identifiers, a computing system that communicates with a given sensor can identify the source of the information. Within the IoT, various devices can communicate with each other and can access data from sources available over various communication networks, including the Internet.

Returning to FIG. 24, in some embodiments of the present invention, based on the one or more drug therapies, the program code selects a pharmacokinetic model (2460). The program code applies the pharmacokinetic model and utilizing the information relating to the patient to determine, for each of the one or more drug therapies, a probability of attaining a PK-PD target associated with efficacy for the patient with the infection (2470). In some embodiments of the present invention, the program code automatically generates rankings, for each of the one or more drug therapies, by ordering each probability of attaining the PK-PD target associated with efficacy for the patient with the infection, for each of the one or more drug therapies, for the one or more drug therapies (2480). The program code displays the rankings (2485). The rankings are a ranked list with the probability of attaining a PK-PD target associated with efficacy for the patient with the infection for each of the one or more drug therapies, ranked in order of predicted efficacy. In some embodiments of the present invention, responsive to the displaying, the program code obtains a third indication comprising designation of a drug therapy from the one or more drug therapies displayed (2488). The program code retains the designation on a memory device (2490).

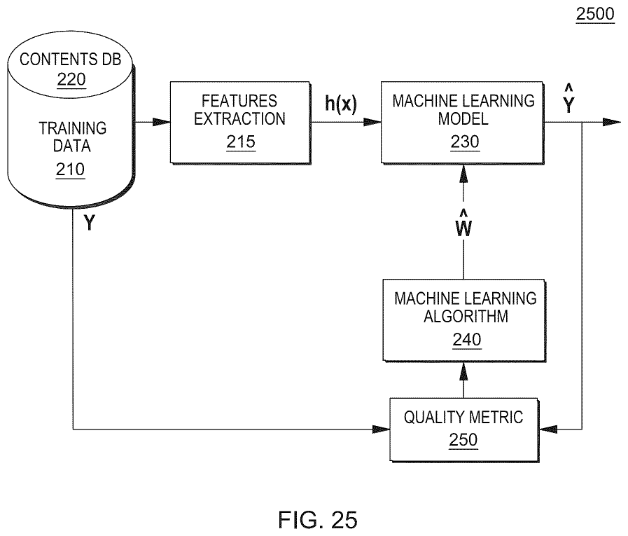

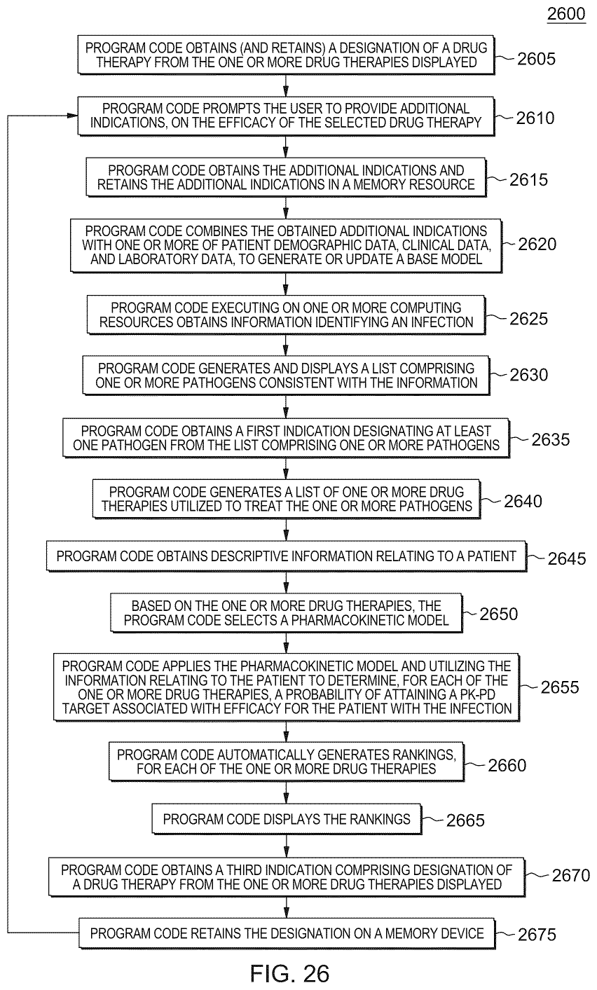

FIG. 25 illustrates various aspects of a machine learning technique that can be utilized in embodiments of the present invention to create and tune a base model utilized in embodiments of the present invention for determining a probability of attaining a PK-PD target associated with efficacy for a patient. In embodiments of the present invention, the base model describes a relationship between patient response and PK-PD target attainment that accounts for patient-specific response modifiers (e.g., previous antibiotic use, age, clearing organ function, etc.). As explained earlier, subsequent to a user providing a third indication comprising designation of a drug therapy from the one or more drug therapies displayed (e.g., FIG. 24, 2488) and the program code retaining the designation on a memory device (e.g., FIG. 24, 2490), the program code in embodiments of the present invention continues to obtain data regarding the success of the drug therapy, from the patient or provider utilizing the application, via an interface. As illustrated in FIGS. 18 and 20, the program code can generate a prompt in the interface to solicit information related to the results of a therapy selected by the user. As illustrated in FIG. 18, some embodiments of the present invention solicit this data at 72 hours after the user selects the given regimen, and also, at a later interval, such as 10 days, as illustrated in FIG. 20. These intervals are merely examples of set time periods at which feedback can be solicited by the program code. As the program code obtains the results of the selected therapies from the users, via a graphical user interface, in some embodiments of the present invention, the program code can identify various features/attributes (e.g., patterns) in this data, and utilize these results as training data 240, to further train a base model to better determine a probability of attaining a PK-PD target associated with efficacy for a patient for future patients. The base model describes a relationship between patient response and PK-PD target attainment that accounts for patient-specific response modifiers (e.g., previous antibiotic use, age, clearing organ function, etc.). In identifying these features/attributes, the program code can utilize various techniques including, but not limited to, mutual information, which is an example of a method that can be utilized to identify features in an embodiment of the present invention. Further embodiments of the present invention utilize varying techniques to select features (elements, patterns, attributes, etc.), including but not limited to, diffusion mapping, principal component analysis, recursive feature elimination (a brute force approach to selecting features), and/or a Random Forest, to select the features. The program code can utilize a machine learning algorithm 240 to train the machine learning model 230 (e.g., the algorithms utilized by the program code, referred to herein as a base model), including providing weights for the conclusions, so that the program code can prioritize various commonalities between patients, such that the program code can learn efficacy patterns based on patient attributes, in accordance with the predictor functions that comprise the machine learning model 230.

As discussed earlier, the base model describes a relationship between patient response and PK-PD target attainment that accounts for patient-specific response modifiers (e.g., previous antibiotic use, age, clearing organ function, etc.). The conclusions can be evaluated by a quality metric 250. Through cognitive analysis, the program code can determine (with increased accuracy based on the repeated use of the model) the probability of attaining a PK-PD target associated with efficacy for a patient, and thus, provide more accurate rankings for various drug therapies. In some embodiments of the present inventions, the personal attributes of the patients can be correlated by the program code such that results of a group of related (based on the analysis of the program code) patients can impact the predicted efficacy of a given drug treatment for a new patient, who shares relevant attributes with this group. Thus, with each new patient, that new patient's data will be used to estimate PK-PD target attainment and the associated the probability of a positive response to the (one or more) drug regimen. As illustrated in certain of the figures, the program code then orders the probabilities and provides them to the user through a graphical user interface.

In some embodiments of the present invention, in addition to the results (e.g., outcome data) provided by the users related to the patients (who may be the users or the clinicians treating the patients can be the users), to tune the base model, the program code utilizes as training data 240 aggregate patient demographic data, clinical data, and laboratory data. The program code can obtain portions of this data from a variety of publicly and privately available data sources. However, patient demographic data is solicited by the program code in some embodiments of the present invention and can be utilized to generate the base model.

FIG. 26 is workflow 2600 that further illustrates the machine learning aspects of some embodiments of the present invention. In an embodiment of the present invention, upon display of the results (e.g., FIG. 15), which can be rankings, where the rankings comprise a ranked list with the probability of attaining a PK-PD target associated with efficacy for the patient with an infection for each of the one or more drug therapies, ranked in order of predicted efficacy, a user (e.g., clinician) selects a regimen and the program code obtains and retains (in a memory) an indication of a selecting of the regimen, via a graphical user interface generated by the program code, on computing device, a designation of a drug therapy from the one or more drug therapies displayed (2605). Program code prompts the user, via the computing device, at one or more predefined intervals, to provide additional indications, via the user interface (2610). The prompts request feedback from the user on the efficacy of the selected regimen (e.g., drug therapy). In some embodiments of the present invention, the user is prompted for this entry whether or not an application comprising the graphical user interface utilized for entry is open on the computing device. The program code can generate a notification on the home screen of the computing device. In some embodiments of the present invention, the program code can communicate with the existing software executing on the computing device to trigger the software to provide the notification. In some embodiments of the present invention, when a user taps the notification, the application which the user utilizes for entry of the requested data is launched automatically on the computing device. In some embodiments of the present invention, the notification is a real-time push message. The program code obtains the additional indications and retains the additional indications in a memory resource (2615). The program code combines the obtained additional indications with one or more of patient demographic data, clinical data, and/or laboratory data, to generate or update a base model (2620). The base model describes a relationship between patient response and PK-PD target attainment that accounts for patient-specific response modifiers (e.g., previous antibiotic use, age, clearing organ function, etc.).

In an embodiment of the present invention, program code obtains information identifying an infection (2625). Based on the information, the program code generates and displays a list comprising one or more pathogens consistent with the information (2630). The program code obtains a first indication designating at least one pathogen from the list comprising one or more pathogens (2635). Based on obtaining the list, the program code generates a list of one or more drug therapies utilized to treat the one or more pathogens (2640). The program code obtains descriptive information relating to a patient, the descriptive information including, but not limited to, an infection acquired by the patient, a pathogen isolated from the patient, a creatinine clearance of the patient, a weight of the patient, and a height of the patient (2645). Based on the one or more drug therapies, the program code selects a pharmacokinetic model (2650). The program code applies the pharmacokinetic model and utilizing the information relating to the patient and the base model, to determine, for each of the one or more drug therapies, a probability of attaining a PK-PD target associated with efficacy for the patient with the infection (2655). In some embodiments of the present invention, the program code automatically generates rankings, for each of the one or more drug therapies, by ordering each probability of attaining the PK-PD target associated with efficacy for the patient with the infection, for each of the one or more drug therapies, for the one or more drug therapies (2660). The program code displays the rankings, wherein the rankings comprise a ranked list with the probability of attaining a PK-PD target associated with efficacy for the patient with the infection for each of the one or more drug therapies, ranked in order of predicted efficacy (2665). As displayed in FIG. 24, responsive to the displaying, the program code obtains a third indication comprising designation of a drug therapy from the one or more drug therapies displayed (2670). The program code retains the designation on a memory device (2675). As illustrated by the path of FIG. 26, the program code the prompts the user, via the computing device, at one or more predefined intervals, to provide additional indications, via the user interface (2610). And hence, the aspects continue to tune the base model and increase the accuracy of the predictions provided by the program code to the user. Thus, data (patient response, PK-PD target attainment, patient-specific response modifiers) from each new patient will be used by the program code to modify the base model.

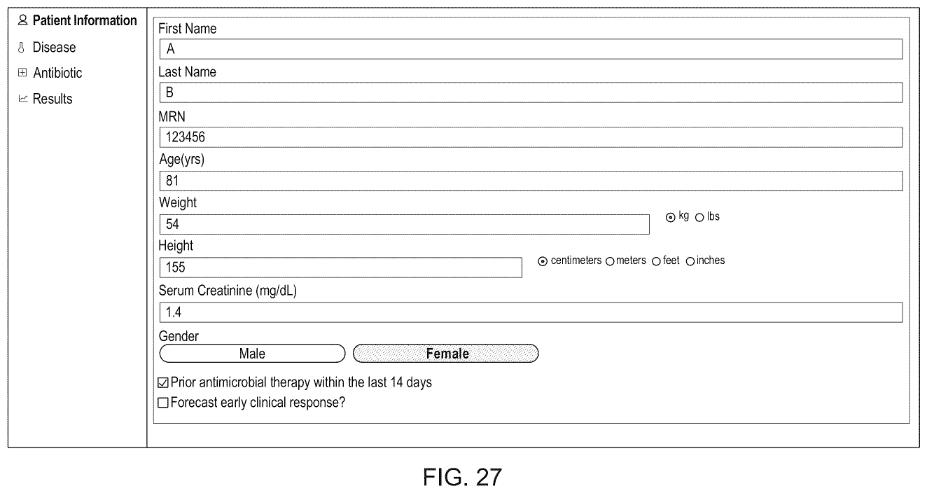

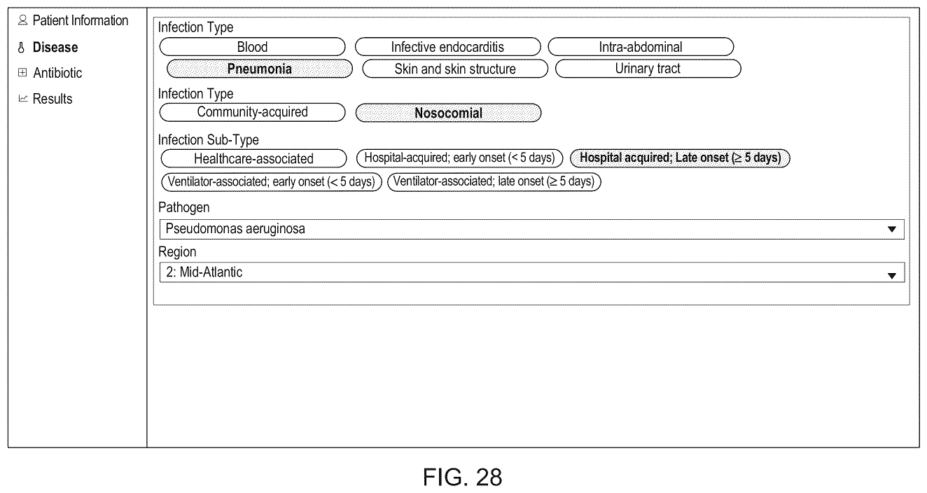

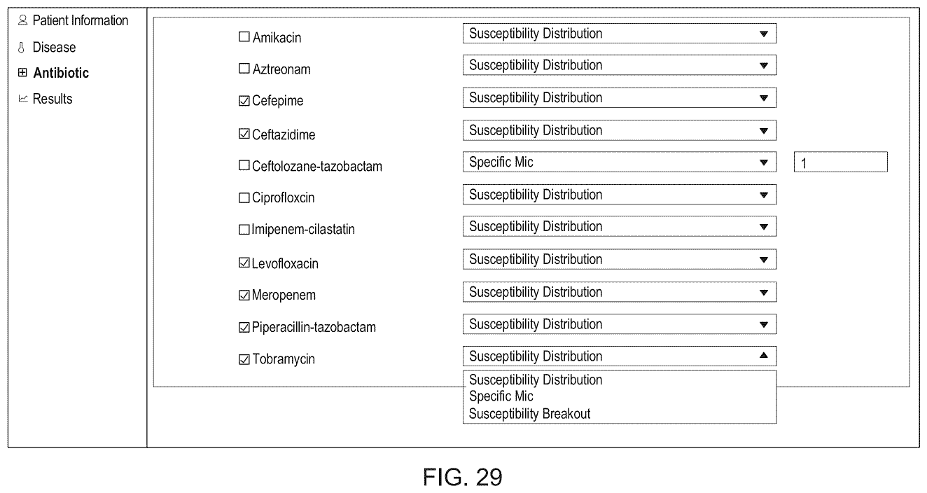

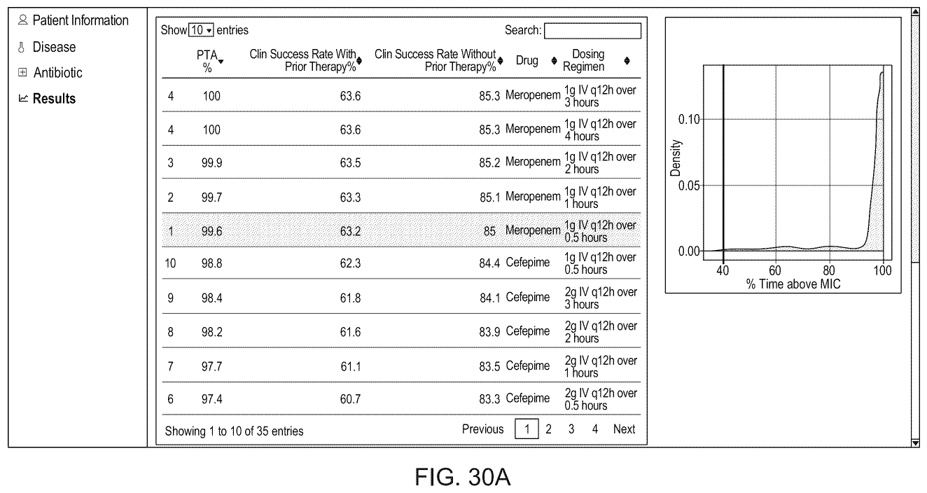

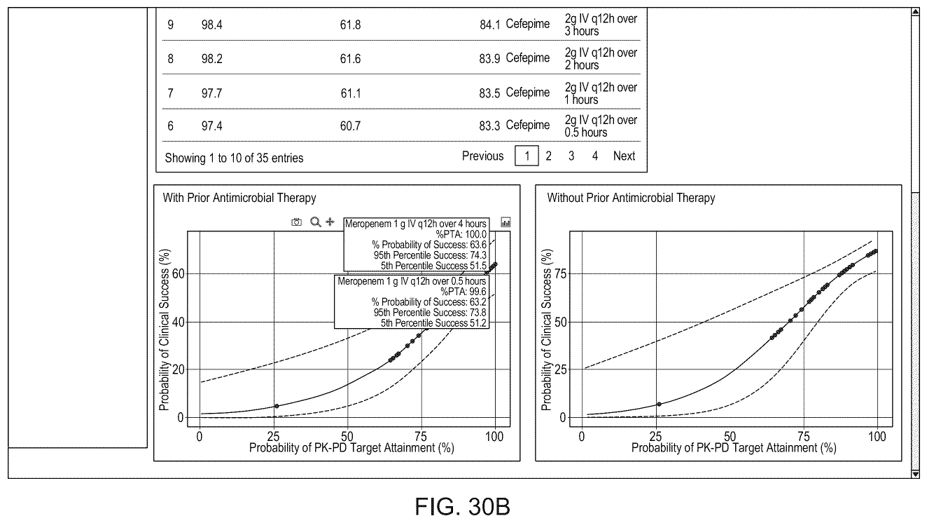

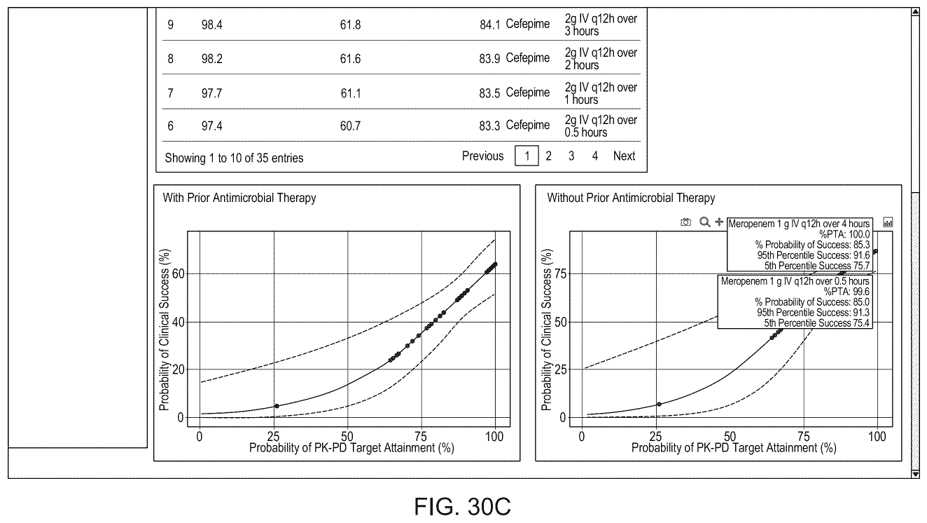

FIGS. 27-30C illustrate examples of graphical user interfaces generated by the program code in embodiments of the present invention on computing devices depict data utilized to generate and update the base model. For example, FIG. 27 illustrates an interface for entry demographic information of an individual utilizing and interface generated by program code executing on one or more processors. FIG. 28 illustrates an interface in which a user identifies an infection. FIG. 29 is a list of drug regimens generated by the program code and the relevant mode in each case. Meanwhile, FIGS. 30A-30C illustrate probabilities of target attainment for one of the recommended drug regimens, the program code having completed an analysis utilizing, in part, the base model.

As discussed earlier, a trigger event refers to a change to one or more records within an EMR system. As will be explained herein, program code executing on one or more processors continuously monitors one or more EMR systems for changes, which can include pre-configured trigger events. When a trigger event occurs, the program code determines whether to update a treatment recommendation based on this event. Because EMRs within the EMR system comprise different types of treatments and medical information, triggers events perceived and analyzed by the program code vary. For example, one type of trigger event is when: 1) a user (e.g., physician, pharmacist) enters a prescription for a listed antibiotic; 2) a culture is received with a listed pathogen; and/or 3) follow up data is received from an existing prescription. FIGS. 31-33 depict workflows illustrating an example of how the program code utilizes this trigger event to determine whether to implement an action in the event of the aforementioned types of trigger events.

The program code that determines and provides recommendations runs as a background process and obtains data indicating that trigger events have occurred within a health system network because of the interconnectivity of various aspects of the system(s). For example, in some embodiments, the program code obtains HL7 feeds (messages) from an EHR system that can include data include, but not limited to, patient census information, orders, and lab results. The program code (including what is referred to herein as the PK-PD compass) comprises FHIR as is authorized by the EHR system (e.g., registered by the EHR system). Security is shared across applications such that a clinician with access to a patient profile in the GUI described herein can also access the same patient's profile in the EHR system. Also, in some examples, the program code can access the EHR system to retrieve any data that is also stored by the program code such that these data can be refreshed and synchronized.

FIG. 31 depicts a workflow 3100 where the program code determines that a trigger event has occurred, and the trigger event is that a prescription has been entered for a listed antibiotic in an EMR system (which the program code interfaces with). In this example, the program code determines that a user (or automated process) has entered a new prescription entered for a listed antibiotic (3110). For example, the program code can recognize a new antibiotic script and new pertinent lab results and text an EMR number to a SMS code. In some examples, the program code, utilizing HL7, can poll prescription and lab tables in the EMR system and based on the polling, identify new matching scripts and lab results and identify the matching medical record. The program code obtains data from the EMR system to complete PK-PD compass calculations (which are described in detail herein) (3120). In some examples, the program code identifies that the new prescription has been entered and based on this identification, the program code performs a lookup in tables that include pathogens as well as treatments.

The program code determines whether the data in the EMR is complete such that the program code can complete the PK-PD calculation (3130). For example, the program code can generate HL7 messages to pull data from an EHR system, including but not limited to Epic or Cerner. The data pulled utilizing the HL7 messaging can include, but is not limited to, prescription (Rx) data, such as drug, dosage, etc. The data can also include blinded (for security purposes) patient data, such as demographic data, including but not limited to serum creatinine and creatinine clearance. The data is blinded because the personally identifiable information is not pulled from the system. The usage of this data by the program code in making a PK-PD calculation is described above in more detail. The data can also include laboratory (test) results and/or suspected pathogens. The data can also include LOS (i.e., the average number of acute days in acute care hospitals compared to expected length of stay), admission data, quality data, drug cost data, etc. Where multiple values exist, the program code can determine which data to utilize based on pre-configured business rules and/or the trained model. In order for the program code to be able to query the EHR system, the program code can utilize a mapping file or register to store mappings of the EHR system (as different heath providers can utilize different systems with different mappings).

If the program code determines that the data is incomplete, the program code generates a link to send to a user (e.g., a pre-configured user such as a physician) with a link directly to an interface (such as those pictured herein) to enable the physician to easily enter the missing data (3137). The interface prompts the user for the data and responsive to the program code continues to evaluate whether the data is in the system (3130). When the program code determines that the data is complete, the program code performs the PK-PD calculation and compares the calculated value to a known optimized PK-PD option (3140). Thus, the program code determines whether the PKPD is optimized (3150). If the calculated PK-PD is optimized, the program code need not take any additional actions and the process terminates. However, if the program code determines that the PK-PD is not optimized (because of the change which triggered this process), the program code notifies a responsible party (e.g., a physician and/or pharmacist) of a new recommendation which is optimized (as the PK-PD compass can calculate a recommendation with the change in the EMR using the method described earlier in this disclosure (3160).

In some examples, whether the PK-PD is optimized in a current prescription is stored in a Boolean value. If the calculated PK-PD is optimized, the program code need not take any additional actions and the process terminates. In some examples, the program code stores the blinded metadata or information and event data. However, if the program code determines that the PK-PD is not optimized (because of the change which triggered this process), the program code notifies a responsible party (e.g., a physician and/or pharmacist) of a new recommendation which is optimized (as the PK-PD compass can calculate a recommendation with the change in the EMR using the method described earlier in this disclosure (3260). In some examples, when the program code determines that the PK-PD is not optimized, the program code generates a recommendation for optimization that includes reasoning for why a recommendation is being made. The program code stores the blinded metadata/information and event data and triggers notification services.

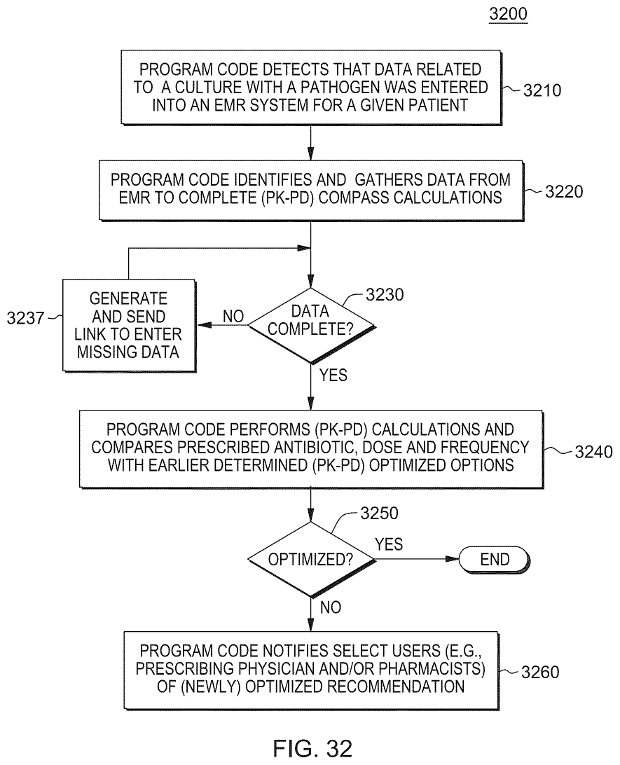

FIG. 32 is a workflow 3200 that is similar to the workflow 3100 of FIG. 31 except that the triggering event is that a culture with the listed pathogen is received by a given instrumentality in an EMR. In this example, data related to a culture with a given pathogen was entered into the EMR system and this entry, into a patient record, is a trigger event for the program code (3210). The program code, in this example, need not identify the pathogen, as in earlier examples, as the culture was received with the pathogen listed. As discussed above, factors such as inter-patient variability in drug exposure, the MIC of the infecting pathogen, and the patient's clinical status, can affect the probability of attaining a PK-PD target associated with efficacy for a drug regimen. The MIC refers to the minimum concentration of a drug therapy that will inhibit the growth of the isolated pathogen. As with the change to the record in the EMR system related to an antibiotic, in this example, the program code determines whether the data in the EMR is complete such that the program code can complete the PK-PD calculation (3230). As in the example of FIG. 31, the program code can generate HL7 messages to pull data from an EHR system, including but not limited to Epic or Cerner. The data pulled utilizing the HL7 messaging can include, but is not limited to, prescription (Rx) data, such as drug, dosage, etc. The data can also include blinded (for security purposes) patient data, such as demographic data, including but not limited to serum creatinine and creatinine clearance. The data is blinded because the personally identifiable information is not pulled from the system. The usage of this data by the program code in making a PK-PD calculation is described above in more detail. The data can also include laboratory (test) results and/or suspected pathogens. The data can also include LOS (i.e., the average number of acute days in acute care hospitals compared to expected length of stay), admission data, quality data, drug cost data, etc. Where multiple values exist, the program code can determine which data to utilize based on pre-configured business rules and/or the trained model. The program code can utilize a mapping file or register to store mappings of the EHR system (as different heath providers can utilize different systems with different mappings).

Returning to FIG. 32, if the program code determines that the data is incomplete, the program code generates a link to send to a user (e.g., a pre-configured user such as a physician) with a link directly to an interface (such as those pictured herein) to enable the physician to easily enter the missing data (3237). The interface prompts the user for the data and responsive to the program code continues to evaluate whether the data is in the system (3230). When the program code determines that the data is complete, the program code performs the PK-PD calculation and compares the calculated value to a known optimized PK-PD option (3240). Thus, the program code determines whether the PKPD is optimized (3250). In some examples, whether the PK-PD is optimized in a current prescription is stored in a Boolean value. If the calculated PK-PD is optimized, the program code need not take any additional actions and the process terminates. In some examples, the program code stores the blinded metadata or information and event data. However, if the program code determines that the PK-PD is not optimized (because of the change which triggered this process), the program code notifies a responsible party (e.g., a physician and/or pharmacist) of a new recommendation which is optimized (as the PK-PD compass can calculate a recommendation with the change in the EMR using the method described earlier in this disclosure (3260). In some examples, when the program code determines that the PK-PD is not optimized, the program code generates a recommendation for optimization that includes reasoning for why a recommendation is being made. The program code stores the blinded metadata/information and event data and triggers notification services.

In FIGS. 31-32 the program code notifies select users (e.g., prescribing physician and/or pharmacists) of (newly) optimized recommendations. In some examples, in both scenarios (and other scenarios), the program code can utilize HL7 to push notes/actions back to an EMR. For example, the program code can (in this manner) create clinical notes in an EMR documenting calculation/result in the EMR. The program code can utilize various methods of electronic communications known to those of skill in the art to alert a prescribing physician of an alternate potential recommendation. In some examples, the program code can provide an alert through an interface, including but not limited to the interfaces portrayed herein. In some examples, the notifications generated by the program code can: 1) alert clinical pharmacists of alternate potential recommendation(s); and/or 2) include a link in the alert to what is being referred to as the PK-PD Compass functionality. A recipient of an alert can automatically view relevant data in the PK PD Compass interface screens (certain of which are depicted in the figures herein). In some examples, when the user clicks the link in the notification, the program code obtains this entry and launches the PK PD Compass interface with data and results pre-populated. Utilizing the user interface, a clinician can manipulate inputs, run additional simulations (utilizing the model), and based on these actions, the program code recommends/provides various conclusions to the clinicians. The program code can store the results of these actions, including the resultant recommendations. In some examples, a clinician utilizing the interface can accept and/or reject results. By accepting the results, the program code can trigger a change in a medical record, for example, by messaging the EHR system utilizing HL7.

Because the program code operates as a service, the program code can routinely maintain existing prescriptions in medical records. This aspect is useful at least because the information utilized to make the PK-PD compass calculations, including the trained model, can change and evolve over time. Thus, the recommended optimized treatment approach to a medical issue can change over time even if aspects in the medical record of an individual remain constant. Thus, in some embodiments of the present invention, business rules are preconfigured (and can be stored on one or more memories accessible to the program code) to enable and/or cause the program code to perform checks on existing prescriptions. For example, in some examples, the program code can perform an analysis on medical treatments prescribed for patients based on aspects, including but not limited to: 1) the passage of a pre-determined amount of time; and/or 2) a business rule specific to a drug and/or pathogen. In some examples, the program code generates an interface that enables authorized users to configure business rules to trigger checks on prescriptions, etc. For example, the program code can enable hospitals (ID and AMS) to create their own rules for follow up and alerts.

Before providing an overview of various aspects of the software as a service (SaaS) and its integration with one or more EHR systems, various scenarios in which this service can be utilized are described below. These scenarios illustrate how the program code can be utilized by different types of users in different physical and figurative places within the medical establishment responsible for determining optimal treatment protocols. A practical application of various of the aspects herein are their accessibility from different entry points. This advantage over existing systems is illustrated in the scenarios that follow.

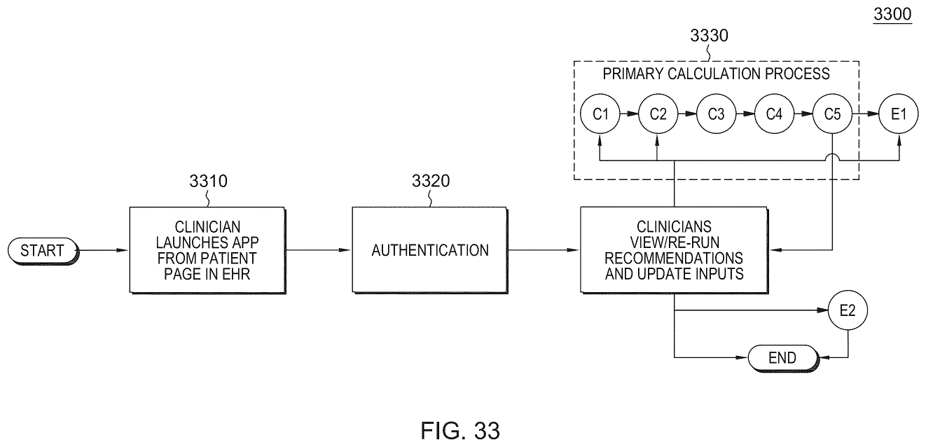

In one example, a clinician launches an interface generated by the program code (e.g., a thick client, thin client or other graphical user interface) from within an EHR with the goal of finding a most effective antibiotics and appropriate dosing and drug regimen for the patient. FIG. 33 is a workflow that illustrates this example. However, certain references in FIG. 33 which include a letter and a number are used in other scenarios to maintain consistency. In the workflow 3300 of FIG. 33, a clinician launches an application (GUI) from a link (e.g., patient page) in an EHR system (3310). The user is authenticated by the program code (3320). In some examples, the program code can be integrated in a third party EHR such that a single sign-on can be utilized and/or the authentication can occur behind the scenes. The clinician can obtain and view recommendations that the program code generated through what is referred to herein (for simplicity, only) as a primary calculation process (3330).

The primary calculation process, in some examples, can be understood as including five aspects, C1, C2, C3, C4, and C5. C1 includes the program code obtaining and storing data for use in calculations. FIGS. 31-32 discuss some examples of the how the program code can obtain these data from the EHR system. The program code can obtain the data through various avenues. For example, based on the authentication (3320), the program code can obtain a token that enables the program code to access these data. The program code can utilize this token, up until its expiration, to fetch data, thus avoiding a call to the EHR. In another example, the program code obtains data based on a clinician prescribing a medication (e.g., a trigger event) that is noted in the EHR. The program code, based on obtaining this trigger, would obtain these data from the EHR. For security purposes, the program code routine that fetches these data can be authenticated by the EHR on a system level, for example. In another example, which is discussed in part in FIGS. 31-32, the program code identifies a change in the EHR (this is based on the program code running as a service and monitoring the EHR for trigger events). The program code, running as a services and monitoring the EHR system receives an HL7 message for a covered event or a covered patient. Based on obtaining the HL7 message, the program code obtains data for subsequent calculations. This process can also include an additional system level authentication for additional security. The data obtained by the program code from the EHR system can include, but is not limited to descriptors related to: a patient, patient encounters (e.g., encounter type (admit, discharge, consult etc.), date/time and care setting (ICU, ER etc.), diagnosis, labs (e.g., including LOINC codes and description, tests ordered, results (e.g., gram stains, culture results including pathogens and MICs, diagnostic reports including cultures and pathogens)), medications, clinician information (ordering clinician). The program code can store metadata including the context of calculation (e.g., use case, date/time, patient, clinician, order details).

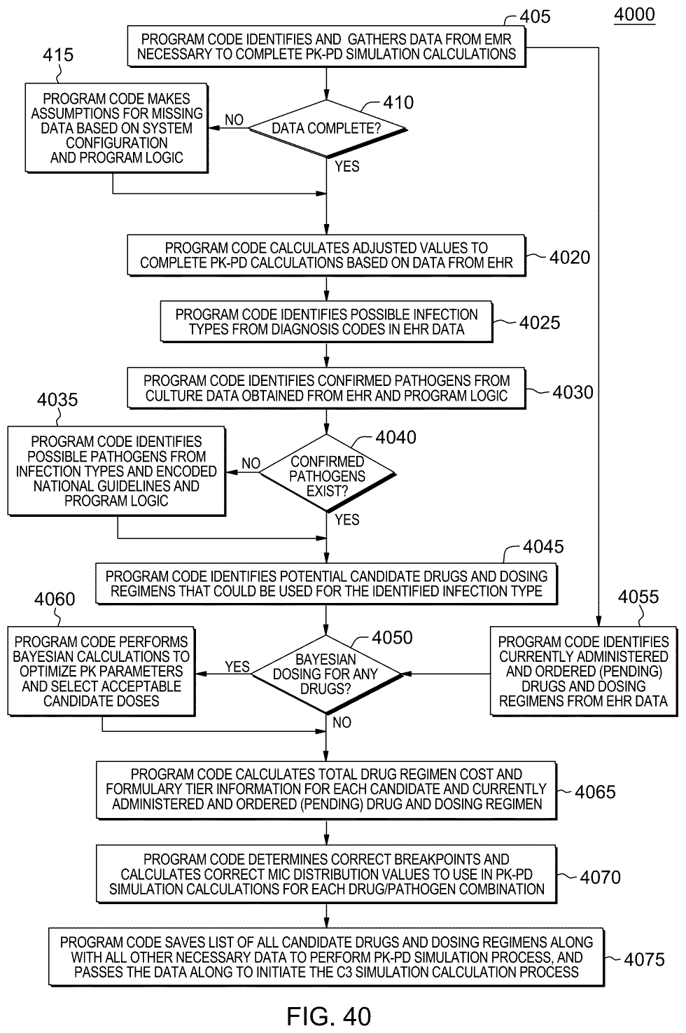

Returning to the example of the calculation process 3300 illustrated in FIG. 33, includes curation of the data by the program code C2. Another example of this process is illustrated in FIG. 40. As part of this calculation, the program code determines the applicability of loading dose based on patient medication administration history and encounter data. To curate the data, the program code obtains the data from the C1 process and from these data can derive and curate additional data. In some embodiments of the present invention, users can update various data through a GUI. In some examples, if any data is updated (added, deleted or changed) by a clinician in GUI screen, then the data can be stored a database, as-is, without any further curation. In the data curation phase (which is illustrated in FIGS. 31-32, in part, when the program code requests additional data), if clinician deselects all data related to clinical input parameters (e.g., all diagnosis or all pathogens etc. have been removed) the program code ca prompt the clinician to select at least one valid value. The program code performs various transformations including curation of data from the C1 process, assumed values, SENTRY data used etc. The program code can store original data and changed inputs (e.g., change by system/user—modified vs system substituted, values pre and post changes, system selections and substitutions (e.g., drug regimen considerations). The program code can also store and apply data transformation logic, including but not limited to, empiric versus culture, fixed dosing versus a Bayesian curation process, and one or more business rules that trigger data substitution and output of a business rule, including but not limited to pathogen selection logic and/or drug regimen selection utilized later in the calculation process (3330). The program code stores all culture result data and metadata needed to support building the antibiogram for a facility/tenant, including, but not limited to, which types of culture were received, culture positive/negative results, S/I/R susceptibility results, MIC distribution data for every culture, locations (floor/unit/type of unit), dates of cultures, primary diagnosis/infection type of cultured patients, temporary regimens based on Bayesian dosing, regimens prescribed outside of the PK PD results produced by the processes described herein. As in the C1 stage of the data processing, the program code can retain metadata (which can be utilized for quality assurance and auditing purposes as well as to train the machine learning algorithms utilized herein).

As aforementioned, an example of the C2 curation process is illustrated in the workflow 4000 of FIG. 40. Referring to FIG. 40, in the example illustrated, program code identifies and gathers data from an EMR to complete a PK-PD simulation (4005). The program code can identify the data for the simulation based on a pre-configuration and/or based on self-guided learning and model building, based on historical data. The program code determines whether the data obtained is incomplete (4010). Based on determining that the data is complete, the program code calculates adjusted values to complete PK-PD calculations (4020). Based on determining that the data is incomplete, the program code generates assumptions for perceived missing data based on system configuration and program logic (which is described herein) (4015). With the assumptions, the program code can complete the PK-PD calculations (4020). The program code identifies possible infection types based on diagnosis codes in the EHR data (4025). The program code identifies confirmed pathogens from culture data the program code obtained from the EHR and program logic (4030). The program code then confirms, based on the data obtained and the analyses described, whether pathogens exist (4040). If there are no pathogens, the program code then identifies possible pathogens from infection types and encoded national guidelines and program logic (preconfigured data and data sources accessible by the program code) (4035). If the program code determines that pathogens do exist (in the culture) and/or the program code identifies pathogens utilizing the data sources and program logic (4035), the program code, based on confirming the pathogens in either manner, can identify potential candidate drugs and dosing regimens that could be used for the identified infection type (4045). The program code then determines whether there is Bayesian dosing for any of the drugs identifies (4050). This information is available via a data source accessible to the program code and/or a model. If Bayesian dosing is utilized, the program code performs Bayesian calculations to optimize PK parameters and select acceptable candidate doses (4060). For drugs where Bayesian dosing is not utilized, the program code identifies currently administered and ordered (pending) drugs and dosing regimens from EHR data (4055). Based on the dosing regimens (4060 and/or 4055), the program code calculates total drug regimen cost and formulary tier information for each candidate and currently administered and ordered (pending) drug and dosing regimen (4065). The program code determines correct breakpoints and calculates correct MIC distribution values to use in PK-PD simulation calculations for each drug/pathogen combination (4070). The program code saves list of all candidate drugs and dosing regimens along with all data to perform a PK-PD simulation process and passes the data along to initiate the C3 simulation calculation process.

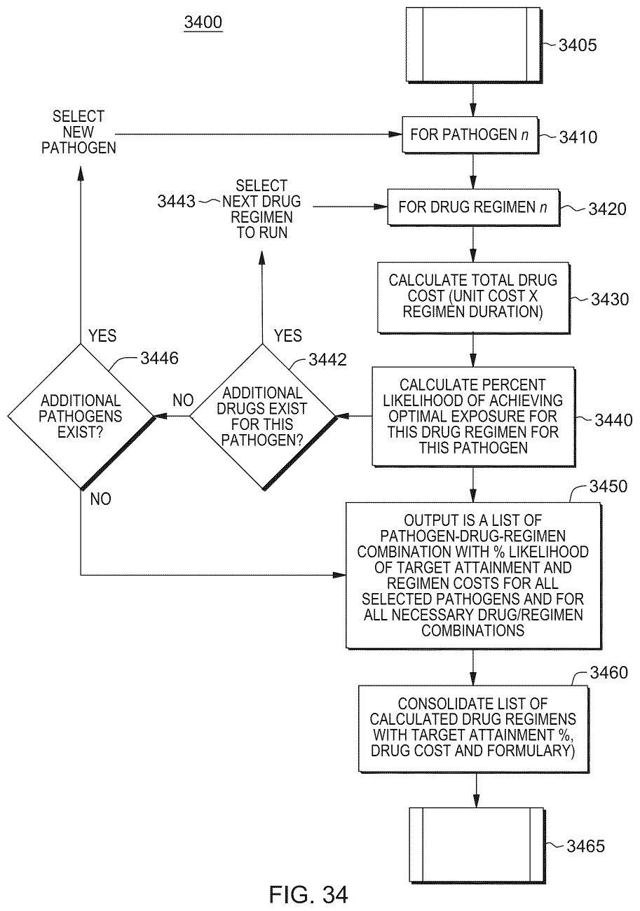

Returning to FIG. 33, once the program code has curated the data (C2), the program code provides the curated set of data for further processing. In this example, the program code applies the trained model discussed earlier in this application. Thus, this C3 calculation stage is discussed in greater detail earlier in this paper. FIG. 34 provides a workflow 3400 of this C3 stage. In this C3 stage, the program code applies the pharmacokinetic model and utilizes the information relating to the patient to determine, for each of the one or more drug therapies, a probability of attaining a PK-PD target associated with efficacy for the patient with a given infection. As discussed earlier, the program code can automatically generate rankings, for each of the one or more drug therapies, by ordering each probability of attaining the PK-PD target associated with efficacy for the patient with the infection, for each of the one or more drug therapies, for the one or more drug therapies. The program code can transmit these calculations to users. At this calculation stage, C3, the program code can apply to the curated data 3405 for each drug regimen (3410) and pathogen combination (3420), a pharmacokinetic model (which can comprise the trained model) and can generate simulations to calculate results. These results can include but are not limited to, the total daily drug cost (e.g., unit cost of a given regimen for the regimen duration) (3430), a likelihood of achieving optimal exposure and dosage by pathogens (3440). In some examples, the implementation of aspects of the invention can be configured to limit the number of simulations performed by the program code. The program code determines after each combination if there are additional drugs for the pathogen (3442) and if there are additional drugs, the program code selects a drug regimen to run (3443). If there are no additional drugs, the program code determines if there are additional pathogens (3446) and will select a new pathogen to evaluate and the process will continue. The program code will determine, for each pathogen and drug, a likelihood of target attainment regimens and provide a list for these combinations (3450). The program code consolidates the list with target attainment percentage drug cost and formulary (3460). The listing with this information is the result 3465 of this stage (C3) of the calculation by the program code. As results 3465, for each pathogen and drug and regimen combination, the program code can provide: % likelihood of achieving optimal exposure, pathogens and MICs, regimen cost, formulary tier, drug amount, and/or patient medication history.

As discussed earlier, the program code ranks the combinations calculated. This ranking is depicted in FIG. 33 as C4. The ranking C4 can also be understood as prioritization by the program code of the regimens. The rules by which the program code prioritizes the results (e.g., FIG. 34, 3465) can be configurable.

The program code prioritizes the results from the C3 portion of the calculation. Table 2 below is an example of target attainment percentage results determined by the program code that are (e.g., FIG. 33, C4) prioritized by the program code. Table 2 are target attainment percentages, a list of single and multi-drug regimens and respective target attainment percentages across all pathogens.

TABLE 2

| | | Target |
|---|---|---|
| Regimen ID | Pathogen | attainment % |
| 1231 | Pathogen 1 | 90% |
| 1232 | Pathogen 1 | 10% |
| 1233 | Pathogen 1 | 50% |
| 1234 | Pathogen 1 | 70% |
| 1235 | Pathogen 1 | 35% |
| 1231 | Pathogen 2 | 70% |
| 1232 | Pathogen 2 | 90% |
| 1233 | Pathogen 2 | 40% |
| 1234 | Pathogen 2 | 70% |
| 1235 | Pathogen 2 | 95% |
| 1231 | Pathogen 3 | 60% |
| 1233 | Pathogen 3 | 90% |
| 1234 | Pathogen 3 | 70% |

Although prioritization can be configurable, in some examples, the program code initially prioritizes based on target attainment percentage for single drug regimens. To this end, the program code generates a data structure, including but not limited the grid illustrated in Table 3, with a list of drug regimen and target attainment percentages for all drug regimens across all pathogens mapped into a grid for each pathogen-regimen identified combination.

TABLE 3

| Regimen | | Pathogen 1 | Pathogen 2 | Pathogen 3 |
|---|---|---|---|---|
| ID | Regimen | [Target %] | [Target %] | [Target %] |
| 1231 | Cefepime | 90% | 70% | 60% |
| | 2 g IV q8h over 0.5 hr | | | |
| 1232 | Ciprofloxacin | 10% | 90% | |
| | 400 mg IV q8h over | | | |
| | 2 hrs | | | |
| 1233 | Levofloxacin | 50% | 40% | 90% |
| | 400 mg IV q8h over | | | |
| | 0.5 hr | | | |
| 1234 | Ceftazidime | 70% | 70% | 70% |
| | 2 g IV q8h over 0.5 hr | | | |
| 1235 | Ceftolozane | | 95% | |
| | 1000 mg IV q8h over | | | |
| | 0.5 hr | | | |

The program code identifies valid (e.g., within a predefined predicted efficacy) drug regimen combinations. To this end, the program code identifies single, double, triple, etc. drugs possible by combining each drug regimens with each other drug regimen. The program code removes combination drug regimens which contain prohibited combinations. Prohibited drug combinations can be pre-configured by an administrator. Prohibited combinations are drugs which cannot be combined with other drugs when given to a patient. A general list can be maintained in a database accessed by the program code.

After determining which combinations are valid, the program code determined target attainment percentages for these combination regimens for each pathogen. The highest target attainment of a drug regimen for a pathogen would be the target attainment percentage of the drug regimen combination for that pathogen (e.g., For Pathogen 1, if Drug regimen A has 9000 target attainment % and Drug regimen B has 80% target attainment then the target attainment % for the 2-drug regimen A & B is 90%). Table 4 is an example of a table containing target percentages that includes a combination of drugs.

TABLE 4

| | | Pathogen | Pathogen | Pathogen |
|---|---|---|---|---|
| | | 1 | 2 | 3 |
| Regimen | | [Target | [Target | [Target |
| ID | Regimens | %] | %] | %] |
| 1231 | Cefepime | 90% | 70% | 60% |
| | 2 g IV q8h over 0.5 hr | | | |
| 1232 | Ciprofloxacin | 10% | 90% | |
| | 400 mg IV q8h over 2 hrs | | | |
| 1233 | Levofloxacin | 50% | 40% | 90% |
| | 400 mg IV q8h over 0.5 hr | | | |
| 1234 | Ceftriaxone | 70% | 70% | 70% |
| | 2 g IV q8h over 0.5 hr | | | |
| 1233-1234 | Levofloxacin/Ceftriaxone | 70% | 70% | 90% |
| | 400 mg IV q8h over 0.5 hr/ | (Highest | (Highest | (Highest |
| | 2 g IV q8h over 0.5 hr | of 70% | of 70% | of 90% |
| | | & 50%) | & 40%) | & 70%) |
| 1231-1232 | Cefepime/Ciprofloxacin | 90% | 90% | 60% |
| | 2 g IV q8h over 0.5 hr/ | | | |
| | 400 mg IV q8h over 2 hrs | | | |
| 1231- | Cefepime/Ciprofloxacin/ | 90% | 90% | 90% |
| 1232-1234 | Levofloxacin | | | |
| | 2 g IV q8h over 0.5 hr/ | | | |
| | 400 mg IV q8h over 2 hrs/ | | | |
| | 400 mg IV q8h over 0.5 hr | | | |

The program code can calculate target attainment percentage for combination regimens across pathogens. Table 5 includes an example of such a calculation.

TABLE 5

| | Pathogen | Pathogen | Pathogen | Average | |
|---|---|---|---|---|---|
| | 1 | 2 | 3 | target | |
| | [Target | [Target | [Target | attainment | |
| Regimens | %] | %] | %] | % | Points |
| Cefepime | 90% | 86% | 60% | (90 + 86 + | 9 |
| 2 g IV q8h | | | | 60)/3 = | |
| over 0.5 hr | | | | 78.66% | |
| Ciprofloxacin | 10% | 90% | | 33.33% | 6 |
| 400 mg IV q8h | | | | | |
| over 2 hrs | | | | | |
| Levofloxacin | 50% | 40% | 90% | 60% | 6 |
| 400 mg IV q8h | | | | | |
| over 0.5 hr | | | | | |
| Ceftriaxone | 70% | 70% | 70% | 70% | 0 |
| 2 g IV q8h over | | | | | |
| 0.5 hr | | | | | |
| Levofloxacin/ | 70% | 70% | 90% | 76.66% | 6 |
| Ceftriaxone | (Highest | (Highest | (Highest | | |
| 400 mg IV q8h | of 70% | of 70% | of 90% | | |
| over 0.5 hr/ | &50%) | &40%) | &70%) | | |
| 2 g IV q8h | | | | | |
| over 0.5 hr | | | | | |
| Cefepime/ | 90% | 90% | 60% | 80% | 12 |
| Ciprofloxacin | | | | | |
| 2 g IV q8h over | | | | | |
| 0.5 hr/400 mg IV | | | | | |
| q8h over 2 hrs | | | | | |
| Cefepime/ | 90% | 90% | 90% | 90% | 18 |
| Ciprofloxacin/ | | | | | |
| Levofloxacin | | | | | |
| 2 g IV q8h over | | | | | |
| 0.5 hr/400 mg IV | | | | | |
| q8h over 2 hrs/ | | | | | |
| 400 mg IV q8h | | | | | |
| over 0.5 hr | | | | | |

The program code can further prioritize the regimens based on target attainment percentage, cost, daily drug amount, dosing interval, and/or infusion time. Various business rules can be configured and associated with each of these parameters by which the program code can prioritize regimens. In some examples, for target attainment percentage, for every infection sub-type and pathogen combination, the program code filters drugs with target attainment percentages in the threshold window and then further sorts (if applicable) based on the configuration high to low or low to high. Additionally, for target attainment percentage, in some examples, if a low and high threshold exist with a sort order (high to low or low to high), then the program code group drug regimens in a band of high and low thresholds and further sort them with highest target attainment percentages at the top based on sort configuration. This threshold target attainment percentage can be configurable. To provide comprehensive results to users, the program code can calculate points (or provide another quantitative measure) for target attainment thresholds to enable ordering combination regimens. In some examples, if two regimens are tied for target attainment, another sort level can be utilized by the program code to determine the ordering. To sort by cost level, the program code can sort the regimens above the threshold attainment percentage and some costing logic can be configured to set a high and a low cost. The program code can further prioritize regimens by drug amount by utilizing a threshold to distinguish a low or high drug amount. Dosing interval and infusing times can also be factors by which to sort regimens based on business rules configured to distinguish preferable conditions to conditions that are not preferable.

In FIG. 33, the final step in the calculation (3330) is processing the output (C5) to provide it back to the EHR in a comprehensive manner. To this end, the program code can retain the reasons for prioritization of each regimen based on configuration, including but not limited to target attainment percentage, cost, formulary, etc. The program code obtains settings that determine the delivery of the results, including delivery method, content and selection for alerts and notifications.

Returning to FIG. 33, at the conclusion of the calculations by the program code (3330), the program code generates a clinical note of a recommendation to send to the EHR system (E1). This recommendation was determined by the program code. In some examples, the program code provides options to a user (e.g., the ranked list) through a GUI and a clinician makes a selection of a regimen and it is the selected regimen that the program code utilizes to send a clinical note to the EHR system. In other examples, the program code automatically selects a regimen which it references to send the note to the EHR system. In some examples, when the program code sends the note, the program code saves, in a database, the note includes a clinical note type (e.g., progress note, consult note etc.), a use case for which the clinical note was generated (e.g., bedside process, CDS hooks, backend process, etc.), content of the note, and date and time of note generation and delivery. As understood by one of skill in the art, a CDS hook refers to an application programming interface (API) that uses a specification that builds on the FHIR core specification. CDS hooks can automatically invoke external Decision Support Services based on events that occur during normal application use within an EHR system. Herein, certain events are referred to as trigger events and these events trigger hooks. A hook generates output to another system, in this case, the application described herein. Upon obtaining this output, the program code can invoke various processes (e.g., calculations C1-C5).

As illustrated in FIG. 33, in addition to the program code sending a clinical note to the EHR system (E1), which the system utilizes to update at least one EHR, the program code also generates and sends a prescribed medication order to the EHR. The program code generates a detailed order which can include, but is not limited to, current and new drug regimens, ordering physician, date/time, duration of therapy etc. To send this order, in some examples, the program code generates at HL7 message.

Thus, the program code sends, in some examples: 1) a consultation note that includes the recommendation generated by the program code (e.g., FIG. 33, E1), and 2) an unsigned medication order based on the recommendation generated by the program code (e.g., FIG. 33, E2).

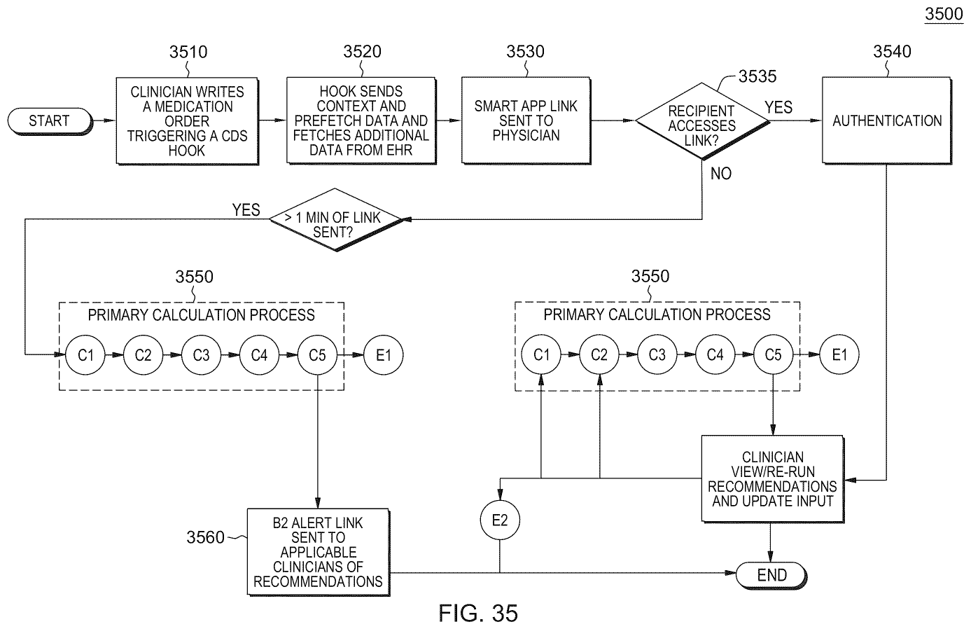

FIG. 35 illustrates how aspects of some embodiments of the present invention can be utilized to provide real-time decision support. In this example, the triggering event is that a clinician creates a medication order for a patient within the EHR, which triggers the program code (running as a service) to launch various processes including the calculations described in FIG. 33. As illustrated in FIG. 35, the triggering event is a clinician (user) utilizing an existing system to enter a prescription (3510). The program code determines that this trigger has occurred and fetches data to be utilized by the PK-PD compass calculations (3520) and also formulates and transmits a link to a physician such that the physician has a shortcut to utilize when accessing the GUI with pre-populated data (3530). As discussed earlier, the program code can fetch the information via an HL7 message to the EHR system. In the workflow 3500 of FIG. 35, there is an expiration associated with the link for security purposes, although this can be configurable. In this example, the physician (the user) can select the link to launch a GUI generated by the program code and can be authenticated via the EHR system (3450), as described in regard to FIG. 33. The physician or clinician user can then interact with the primary calculation process as it is performed by the program code (3550), which includes elements C1-C5 described earlier. Alternatively, if the physician does not select the link, the program code, after the expiry of a pre-defined period of time, can automatically perform the primary calculation process (3550). Whether the calculation is performed completely automatically by the program code or with user interaction, the program code can generate an unsigned medication order (E2). As discussed when describing FIG. 33, the program code fetches all details and stores patient and clinical information (C1), curates the data (C2), performs the calculations by applying the model(s) to generate recommendations (C3), prioritizes and sorts regimens (as determined by the model(s)) (C4), and processes the results, which can include providing (and/or saving) reasons for recommendation(s), comparing current and previous regimens, and/or packing alerts (C5).

If the physician is not working through the GUI, in addition to sending the order (E2), the program code can also send an alert with the recommendation to the physician (or other designated recipients) (3660).The system should receive the trigger and details required for verifying if the medication is for a covered drug or a covered patient. In some examples, the program code determines if the hook (medication entry) is covered and only if covered does the process continue. Thus, in some examples, the program code sends a clinical note of recommendations to the EHR (E1) and sends a new medication order and/or cancels an existing order in the EHR based on clinician selection in the GUI (provided by the program code and illustrated in FIG. 35). The hook referenced herein can comprise a setup for interoperability between program code of the examples described herein and an EHR system. In some examples herein, at least certain aspects of the operability between the EHR system and the combined software and/or hardware systems described herein are provided by REST services. Specifically, REST APIs are utilized as endpoints on servers that enable other resources to access applications associated with the APIs that are deployed on the servers. REST APIs may be available from each of the individual servers in a multi-server environment that includes the EHR system, providing endpoints to applications executing on the various servers. In some examples, a REST endpoint on a server executing the program code described herein provides an access point for the EHR system.

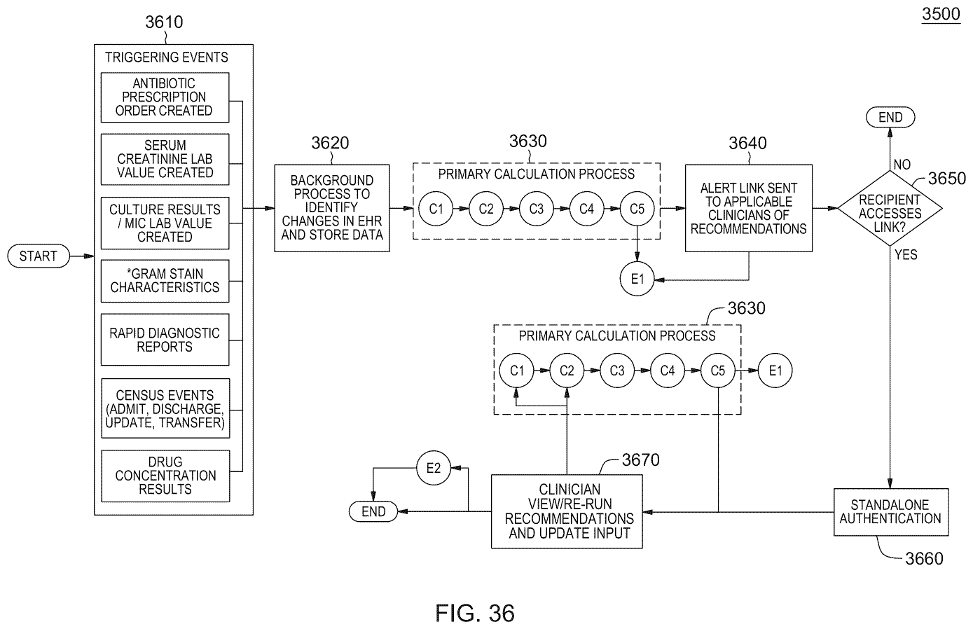

Because the program code executes as a service, there are arguably an unlimited number of events that could trigger a calculation via the PK-PD compass. The program code constantly receives unsolicited real time notifications from the EHR on patient census events, order notifications, and/or lab results. Each of these events could necessitate a regimen change. These events can include, but are not limited to, the creation of an anti-biotic prescription order, the creation of a serum creatine lab value, obtaining culture results (MIC lab values created), obtaining germ stain characteristics observed, obtaining rapid diagnostic reports received, census events (admissions, updates, transfers, discharges, etc.), obtaining drug concentration results, etc. FIG. 36 illustrates a workflow 3600 where a background event triggers the program code to evaluate and recommend regimens.

Referring to FIG. 36, the program code determines that a trigger event has occurred (3610) within an EHR system and/or a system that interfaces with the EHR system (which will impact data in one or more of the databases accessed by the EHR system). The program code, for each change that is configured as a trigger event, the program code obtains data utilized for the calculations from the EHR, including via HL7 message (3620). However, as the change was observed based on the program code running as a service (e.g., in the background), as part of the data gathering, the program code obtains changes in the EHR data (3620). The program code performs the calculation process (e.g., C1-C5, as described earlier) (3630) and generates a clinical note of a recommendation to send to the EHR system (E1). Because the trigger is in the background, a clinician who would act of the recommendation may not be actively engaged with any system and thus, in real-time, when the program code generates the recommendation (E1), the program code also sends an alert of the recommendation which can include a link (3640).

As discussed above, because the trigger events are observed by the program code running an a background process, a clinician whose treatment plan for one or more patients may be impacted by the calculations by the program code based on the data changed in the trigger event is likely not engaged with the system through a GUI and is certainly unlikely to be engaged directly with a screen that recommends a treatment or regimen for a given pathogen. Thus, when the program code determines that a trigger event has occurred, determines that the event is a covered event, and performs simulations and calculations based on the event, the program code also alerts relevant parties impacted by the resultant recommendation (3640). In some examples, the targets for the alerts generated by the program code are based on pre-defined logic in a configurable alerts table. The configurable aspects can include, but are not limited to, recipients (e.g., users, prescribers, distribution lists), preferred types of alerts (delivery methods), content of alerts based on the specific alert reason or potential severity, and/or clinical note configurations (e.g., alert type, tenant, and facility).

Types of alerts generated by the program code and transmitted by the program code to interested parties can include, but are not limited to, text notifications and/or email notifications. Both these email and text notifications can leverage a HIPAA compliant Omni channel platform. In some examples, the alerts generated contain the context of a target (e.g., user role, distribution list, alert type etc.) and the context of the relevant patient record (e.g., patient and prescription information). In some examples, the alerts generated by the program code include a link to launch a GUI (screen) and/or an option to dismiss the alert so no reminders are received in the future for that specific alert notification.

As part of the alert process (3640), the program code can: 1) generate and send the alert; 2) create and send a clinical note (e.g., comprising details on the alert) to the EHR (e.g., E1) after the alert is triggered; 3) log a record of the alert for auditing and reporting purposes containing the contents of the alert and associated metadata; and/or 4) flag a specific patient/prescription as “alert sent” such that this detail would be viewable in a GUI utilized by a clinician. The program code can also log errors associated with these alerts.

The workflow 3600 continues when the user receives and activates the link and ends if the user does not receive and follow the link (3650). Based on accessing the link, the program code authenticates the user (3660). The user can then view the recommendation that was generated behind the scenes and continue to view and update inputs (3670) until a recommendation that the user accepts is generated by the program code performing the calculations (e.g., C1-C5) (3630). The program code can generate an unsigned medication order (E2) based on the user ending the user's interaction with the calculation process and accepting a recommendation with which to proceed.

Because the program code in various embodiments described herein can run as a service and as a background process, and the program code is integrated with various systems utilized in health-care environments, in some embodiments of the present invention, a clinician can utilize the systems described herein to perform a multiple patient review. An example of this activity would be if a given clinician, including but not limited to a clinical pharmacologist, in a healthcare setting, including but not limited to a hospital, wanted to view orders pending review in the GUI generated by the program code described herein. Embodiments described herein could enable the clinician to launch a GUI comprising the program code described herein from an EHR system of as a standalone. The user (clinician) could scroll through various patients, select individual patients, either in the (proprietary) GUI or an EHR system, and trigger the calculation process (e.g., C1-C5). If the clinician chooses to perform the review through the GUI, the clinician can launch the GUI (viewing, for example, a multi-patient review screen) and review all outstanding orders. The program code can authenticate the clinician login with the EHR using the security framework of the existing systems which with the program code is integrated to enable sign on or the user can authenticate to the application directly. Based on gaining access as an authorized user, the program code fetches data and displays a list of patients with all of their orders (per pre-configured business ruled). For example, a given patient can be displayed by the program code if the patient has at least one order for a covered antibiotic stored in a database accessed by the program code (including a proprietary database that serves as a backup to the application comprising the program code). The program code displays the list for review to the user. In some examples, when the clinician selects an individual patient, the program code generates and displays a detailed view of that patient's data. To prevent users from overwriting each other's work in the system, the program code can lock a given patient's record (and generate a characteristic on the screen to indicate the lock to all concurrent users). In some examples, the program code can indicate which records are being edited by which users to other users.

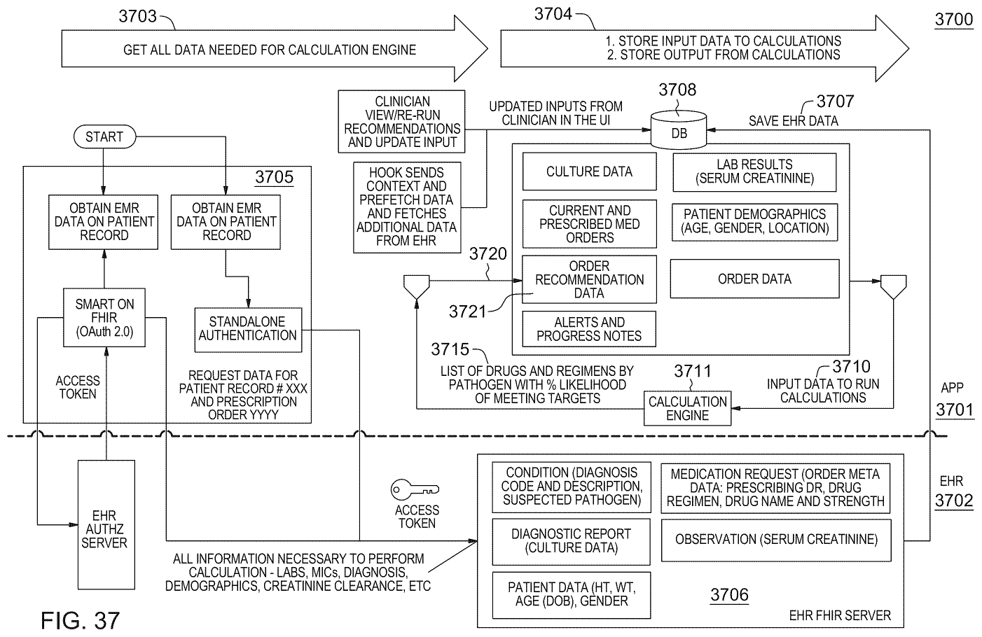

The calculations performed by the program code include aspects C1-C3, which are included in FIGS. 31-36. FIGS. 37-39 provide additional detail related to these individual aspects.

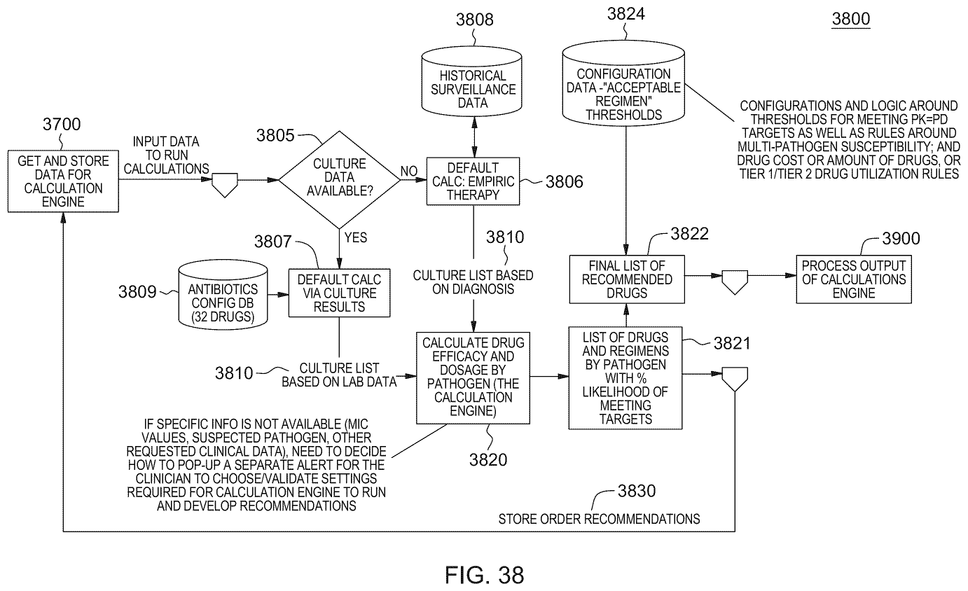

FIG. 37 provides further illustrations of the C1 aspect of various calculations performed by the program code. In this aspect, as discussed earlier, the program code comprising the application 3701 interfaces with an EHR system 3702 to obtain and store data utilized to recommend a regimen or change in regimen. FIG. 37 illustrates a possible configuration of different data within the different (interacting systems) as well as how the systems interact securely, including by utilizing a token. At the conclusion of the aspect, the program code has obtained (from the EHR system 3702) (3703) and stored (in a database of the application 3701) the data that is utilized by the program code in further calculations (3704). In the workflow 3700 depicted in FIG. 37, program code executing on one or more processors (which is part of a proprietary application 3701 as opposed an EHR system 3702), obtains EMR data from the EHR system 3702 using various methods of connectivity and authentication, including but not limited to, connecting to a server utilizing a token as a background process and/or authenticating directly by utilizing a standalone version of the application 3701 (3705). The program code obtains data from one or more FHIR servers 3706 on the EHR system 3702 (3705). The program code stores the data obtained in at least one database 3708 accessible to the applications 3701 (as explained earlier, the data saved in resources of the proprietary system herein may not include personally identifiable data) (3707). The program code inputs the stored data into a calculation engine 3711 (comprised of the program code) (3710) and the program code of the calculation engine (which is illustrated in FIG. 38), outputs a list of drugs and regimens with a percentage likelihood of meeting targets (3715). From this list, the program code can generate an order recommendation 3721, which the program code saves in the at least one database 3708 (3720).

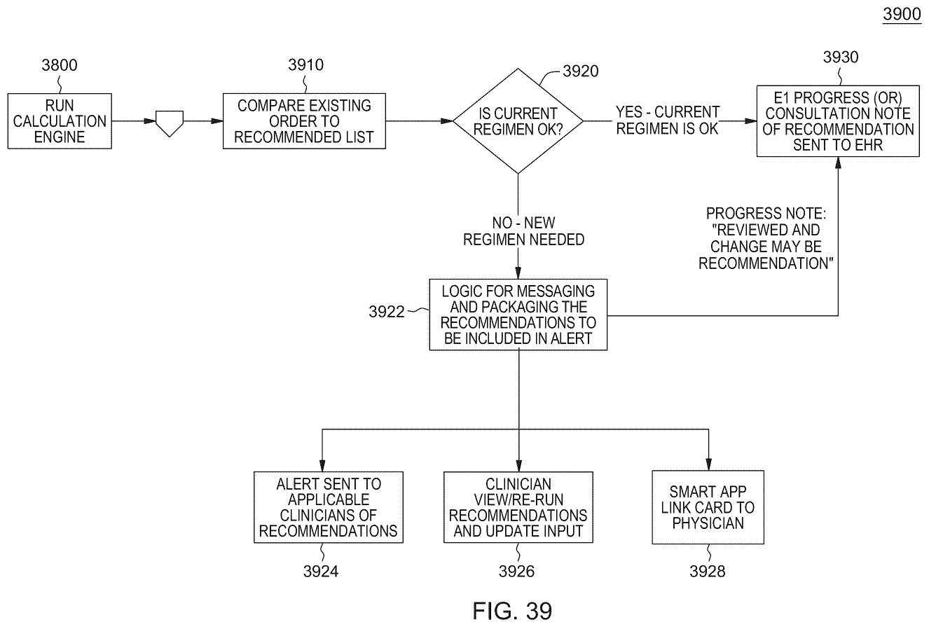

FIG. 38 further illustrates the C2 aspect of the calculations discussed herein in which the program code (executing on one or more processors) curates the data obtained by the program code. The final aspect of the workflow 3800 of FIG. 38 proceeds to the workflow 3900 of FIG. 39 (C3). As illustrated in FIG. 38, the program code obtains the data that was earlier fetched and stored by the program code (see, FIG. 37, 3700), obtains available culture data (3805), if available, or otherwise defaults to empiric therapy data 3806 stored in a database accessible to the program code 3808, including a database comprising historical surveillance data. If culture data is available, the program code obtains antibiotic configurations from one or more databases 3809 and obtains lab results 3807 which comprise default calculations based on the culture results by utilizing the antibiotic data. The program code generates a culture list either based on the default data 3806 or based on the lab results 3807 (3810). The program code calculates drug efficacy and dosage by pathogen (based on the culture list) (3820). When the program code calculates the efficacy, if specific information is not available (e.g., MIC values, suspected pathogen, etc.) the program code can be configured to provide an alert to a clinician to provide the information such that the calculation can proceed. As an output of the calculation (3820), the program code generates a list drugs and regimens by predicted efficacy (applying the models discussed earlier) 3821 and generates a final list of results 3822. The program code stores the order recommendations generated by the program code (3830). The final list 3822 is informed by reconfigured information that is stored in a database accessed by the program code 3824. FIG. 38 displays only an application-side of these examples because the aspects described in the workflow 3800 are enacted by the program code of the application. FIG. 39 similarly illustrates a workflow that is performed by program code in a proprietary application as opposed to in the EHR system (with which the application communicates).

The workflow 3900 of FIG. 39 illustrates the C3 stage of aspects of some embodiments of the present invention. In this C3 stage, the program code applies the pharmacokinetic model and utilizes the information relating to the patient to determine, for each of the one or more drug therapies, a probability of attaining a PK-PD target associated with efficacy for the patient with a given infection. The program code obtains results from the calculation engine (see, FIG. 38, 3800) and compares an existing order to the recommendation list generated by the program code (3810). The program code determines if the current regimen remains a recommendation (3820). As discussed earlier, an impetus for the program code determining regimen efficacies is based on a trigger event occurring the EHR system (e.g., a change to an EMR) and the program code, based on determining that the trigger event has occurred, fetches and stores data to ultimately make a recommendation regarding a regimen. Thus, in this workflow 3900, the program code determines if an existing order (test, medication, etc.) would attain a desired efficacy despite the change in data.

Returning, the FIG. 38, whether the program code determines that the current regimen is acceptable (would be recommended based on efficacy) or not (3920), the program code generates a clinical note of a recommendation to send to the EHR system (e.g., E1) (3930). However, in this example, if the program code determines that the current regimen is no longer recommended, the program code can take various actions to alert individuals utilizing various parts of the application, the EHR system, and external systems, of the change. In the event of a new regimen being recommended, the program code obtains stored (pre-configured) logic for packaging the recommendation. Based on this logic, the program code can send an alert to a clinician with the recommendation (3924), can display the recommendation to a clinician in a GUI of the application such that the clinician can re-run the recommendation process/software (3926), and/or transmit a link to the user such that the user can utilize the link to connect to the application via a GUI, accessed with the link, with the relevant data pre-populated in the interface (3928).

As demonstrated in the figures herein, the program code: 1) communicates with an existing EHR system; 2) listens for events based on the connectivity to the EHR system; 3) collects information in to make various decisions based on various calculations; and 4) presents a recommendation for a given issue. The program code can deliver this recommendation as an alert. The delivery of the alerts by the program code can be governed by logic related to one or more of the contents of the alerts and/or logic related to the intended recipients of the alerts.

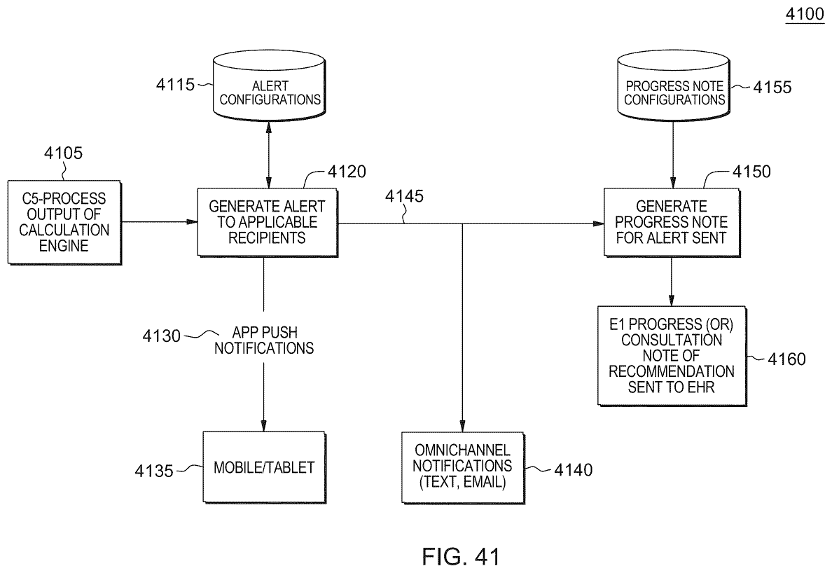

FIG. 41 is a workflow 4100 that illustrates the alert process. The program code that provides the alert (which can be a separate module and/or part of any other part of the system) obtains the output of the calculation (C5) 4105 (4110). Based on the output 4105, as well as alert configurations (saved in an accessible data source) 4115, the program code generates alerts to applicable (based on the output 4105 and configurations 4115) alerts (4120). The alert configurations include parameters including, but not limited to, type of user, distribution list, methods of alerts by alert type, tenants, and/or facilities, etc.

The alerts generated by the program code (4120) are customized based on the alert configurations 4115 as well as the output 4105. For example, an alert can include context of the target of the alert (e.g., user role, etc.). Configuration elements of the alerts can include, but are not limited to, recipients (users/prescribers/distribution lists), preferred types of alerts (delivery methods), content of alerts based on the specific alert reason or potential severity, clinical note configurations (e.g., configurations for type of clinical notes by alert type, tenant and facility).

If the alerts are being delegated to a standalone application by the program code, the context can include authentication to access the alert capabilities of the application. For example, the program code can generate the alerts as push notifications for various applications in the technical environment (4130). The alerts can also include context related to the relevant patient records, including but not limited to, the records number of the patient and a prescription order, if relevant. As illustrated in FIG. 41, based on the configuration, the program code can deliver the alerts via one or more applications to a computing node (e.g., mobile or tablet 4135) (4130) and/or to a messaging system that handles notifications in different channels (e.g., text, email) 4140 (4145).

The examples herein include various features that are part of a feedback loop so that the program code and therefore, the functionality of the system as a whole, can continually improve. Thus, based on the alerts, the program code continues to monitor the EHR and other connected systems and, as illustrated in FIG. 33, the program code generates a clinical note of a recommendation to send to the EHR system (E1) (4150). In this example, the note is a progress note (4160). The note can contain the details of the alert sent. The program code obtains a configuration for the progress note 4160, in this example, by utilizing a saved progress note configuration from a data source 4155. The data source 4155 includes configurations for notes that include parameters such as type of progress, notes by alert type, tenant, and/or facility, etc.

The alert mechanism (B2, FIG. 41) can obtain notifications from the C3 stage of the calculations and can generate alerts based on the logic in the alert configuration table/source 4115 (4120). The alerts can include but are not limited to text notifications and/or email notifications 4140. In some examples, the email notifications leverage the HIPAA compliant Omni channel platform (e.g., 4140). As aforementioned, the alerts can contain the context of the target (user role, distribution list, alert type etc.) and the context of the patient record (patient and prescription information). The alerts can include a link to launch a responsive UI and another link to dismiss the alert, so no reminders are received in the future for that specific alert notification.

As illustrated in FIG. 41, as part of the alert mechanism, the program code: 1) generates and sends alerts; 2) generates clinical notes and sends these clinical notes to the EHR (E1) after an alert is triggered. In some examples, the program code logs a record of each alert for auditing and reporting purposes containing the contents of the alert and associated metadata. The program code can flag the specific patient and/or prescription order as “alert sent” which should show up in the graphical user interface. The program code can also log failed alerts for further analysis (e.g., by a support team). The logged alert error can include the context of the alert (patient/prescription information for which an alert was generated). If a clinical note cannot be generated or sent, all information with the context of the note can be stored by the program code. Additionally, in some examples, the program code stores separate error information that should be stored with respect to an alert generated successfully but could not be delivered via the preferred channels (text/email).

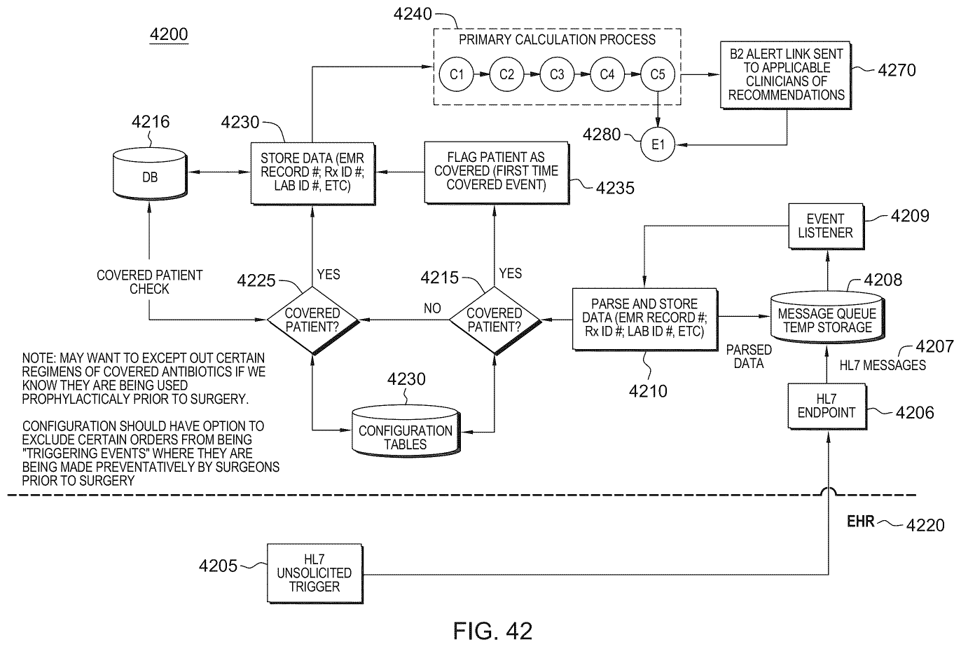

As discussed herein, program code in embodiments of the present invention runs as a background process and identifies changes in an EHR and stores data. FIG. 42 illustrates aspects of some examples of this background process. Please note that in this process, program code calculations (C1-C5), program code sending a clinical note to the EHR system (E1), and the alert process (B2, which was just depicted in FIG. 41), are also included. Thus, one can contextualize other processes described herein.

Referring to FIG. 2, in this workflow 4200 examples, program code executing on one or more processors obtains unsolicited triggers from an EHR 4220 (4205). The triggers can be HL7 feeds (HL7 messages 4207 sent to an HL7 endpoint 4106 and passed to a queue 4208) from the EHR system 4220 and the program code can store the triggers (e.g., messages 4207) in a repository 4208 as it works through each trigger. Alternately, the EHR can be configured to store the triggers in a repository such that the program code can access the triggers in that repository. An administrator can register the systems embodied in FIG. 42 (above the line indicating the EHR 4220) within the technical infrastructure of the site (e.g., hospital). These triggers can include, but are not limited to, patient census information (e.g., admit, cancel admit, transfer, discharge, update), medication orders (e.g., new order, change order, discontinue or cancel order), observations including lab orders and diagnostic reports (e.g., order message, pharmacy/treatment encoded order, results). The program code obtains the HL7 message 4207 and parses and stores the message in a (temporary) location (e.g., a message queue 4208) such that the program code can evaluate whether it is a covered event (4210). Program code comprising a listener 4209 monitors the message queue 4208 to provide the message data to program code comprising a parser that will parse these data (4210). The program code determines whether each trigger (e.g., HL7 message 4207) indicates a covered event (4215) and/or a covered patient (4215), based on configurations stored in a data source, including but not limited to, configuration tables 4230. A non-limiting example of a covered patient definition is a patient that has at least one covered antibiotic order or pathogen in the triggering event or an earlier event (obtained via HL7 message by the program code).

In some examples, the configuration, and thus, configuration tables 4230 that guide the program code decision on whether a trigger is a covered event and/or a covered patient, can be edited and updated by an administrator (e.g., via a GUI). In some examples, the configuration functionality provides an obtain to exclude certain orders from being “triggering events” (e.g., where they are being made preventatively by surgeons prior to surgery). Additionally, certain regimens of covered antibiotics can be excluded if identified as being used prophylactically prior to the surgery. In certain situations (as illustrated in FIG. 41), when the program code identifies a covered event and/or covered patient, the program code sends an alert (B2) (4270). In determining whether a patient or event is covered (4225, 4215), the program code, in advance, parses the stores the data from the trigger (e.g., EMR record number, prescription number, lab identification number, etc.). The program code also can consult a data source where past events have been stored to make this determination.

Returning to FIG. 42, in some examples, when the program code determined that a trigger comprises a covered event or patient (4225, 4215), the program code stores the covered event or patient record in a data source 4416 (4230). In this example, once the record is stored in the data source 4416 (4230), the program code can clear the record from the temporary storage or message queue 4208. The program code also determines if the patient designated in the message (which has been determined to be a covered event) is a first-time patient and if so, the program code flags the event as a first-time covered event (4235). In some examples, for a covered event, the program code performs a refresh via the FHR in the C1 stage of the calculation in ensure the program code is retaining the most current information in the database 4216.

In some embodiments of the present invention, for a covered patient, the program code makes a call (e.g., to an electronic medical records system) to get current snapshot of all clinical information (medications, labs, vitals, and diagnoses, etc.), incrementally stores events for the covered patient including non-covered events going forward, and if events are received for the same patient after a discharge, the program code can track the new admission as a separate admission (information from earlier admissions can remain available and selectable through a GUI), if relevant, information about the hospital stay (including data points to calculate outcome metrics).

When the program code determines that a trigger comprises a covered event, the program code initiates the calculation processes (C1-C5). The program code can determine, based on the resultant calculation, whether to send an alert (B2) (4270), and whether to write a clinical note (E1) (4280).

Examples herein include computer-implemented methods, computer program products, and computer systems where program code executing on one or more processors determines that a trigger event related to a patient, wherein the patient record comprises an order for a current drug regimen, has occurred in an electronic health record (EHR) system communicatively coupled to the one or more processors. Based on obtaining the notification, obtaining, from one or more electronic medical records (EMRs) stored in the EHR system communicatively coupled to the one or more processors, descriptive information relating to a patient, the descriptive information comprising one or more data elements selected from the group consisting of: an infection acquired by the patient, a pathogen isolated from the patient, a creatinine clearance of the patient, a weight of the patient, and a height of the patient. Based on the one or more drug therapies, the program code selects a pharmacokinetic model. The program code applies the pharmacokinetic model and utilizes the information relating to the patient to determine, for each of the one or more drug therapies, a probability of attaining a PK-PD target associated with efficacy for the patient with the infection. The program code automatically generates rankings for each of the one or more drug therapies, by ordering each probability of attaining the PK-PD target associated with efficacy for the patient with the infection, for each of the one or more drug therapies, for the one or more drug therapies, where the rankings comprise a ranked list with the probability of attaining a PK-PD target associated with efficacy for the patient with the infection for each of the one or more drug therapies, ranked in order of predicted efficacy. The program code determines if the current drug regimen comprises a probability above a preconfigured threshold. Based on the determining and the rankings, the program code generates a recommendation for a new order.

In some examples, the program code determining if the current drug regimen comprises the probability above the preconfigured threshold comprises the program code determining that the current drug regimen comprises the probability above the preconfigured threshold, and the generating comprises: the program code generating a clinical note of a recommendation, where the clinical note of a recommendation comprises the new order, and where the new order is an order for the current drug regimen, and the program code transmitting the clinical node of the recommendation to the EHR system communicatively coupled to the one or more processors.

In some examples, the program code determining if the current drug regimen comprises the probability above the preconfigured threshold comprises the program code determining that the current drug regimen does not comprise a probability above the preconfigured threshold. The program code generating including the program code generating a clinical note of a recommendation, where the clinical note of a recommendation comprises the new order, and the program code transmitting the clinical node of the recommendation to the EHR system communicatively coupled to the one or more processors.

In some examples, the program code generating the recommendation for a new order comprises: the program code generating an unsigned medication order based on the ranked list, the unsigned medication order comprises the new order.

In some examples, the program code generating the recommendation for a new order comprises: the program code transmitting an alert to at least one user, the program code obtaining a response to the alert, where the response comprises a selection of a designation of a drug therapy from the one or more drug therapies comprising the ranked list, where the drug therapy designated comprises the new order, and the program code generating an unsigned medication order comprising the new order.

In some examples, the program code transmitting the alert comprises: the program code generating a message comprising a link to launch a graphical user interface, where the one or more processors automatically display the rankings in the graphical user interface upon selection of the link by a user receiving the message, and the program code transmitting the message to at least one user pre-defined to receive the message, where the user contact information is saved in a database communicatively coupled to the one or more processors, where the response the selection of the designation of a drug therapy is performed by the user in the graphical user interface.

In some examples, the program code transmitting the alert comprises: the program code displaying the rankings, in a graphical user interface, where the response the selection of the designation of a drug therapy is performed by the user in the graphical user interface.

In some examples, the trigger event comprises an update to at least one field of at least one electronic medical record in the EHR system.

In some examples, the trigger event is selected from the group consisting of: entry of a prescription for a given antibiotic, reception of a culture with a given pathogen, and entry of data comprising additional information about an existing prescription.

In some examples, the program code retains the recommendation on a memory device. The program code prompts, through a user interface, a user to provide data indicating an actual efficacy of the drug therapy as utilized by the patient with the infection at one or more predetermined intervals after obtaining the recommendation. The program code obtains, responsive to the prompting, the data indicating the actual efficacy of the drug therapy.

In some examples, the program code generates or updates, based on data comprising the data indicating the actual efficacy, a base model, where the base model describes a relationship between given patient response and PK-PD target attainment that accounts based on patient-specific response modifiers.

In some examples, the data further comprises data selected from the group consisting of: patient demographic data, clinical data, and laboratory data.

In some examples, the program code selecting the pharmacokinetic model comprises: for each of the one or more drug therapies, the program code determines a class for a PK-PD index. Based on determining that a drug therapy of the one or more drug therapies is in a first class, the program code selects a pharmacokinetic model, where applying the pharmacokinetic model comprises evaluating total drug exposure in a 24-hour period, for the drug therapy, to determine the probability of attaining a PK-PD target associated with efficacy for the patient with the infection. Based on determining that a drug therapy of the one or more drug therapies is in a second class, the program code selects a pharmacokinetic model, where applying the pharmacokinetic model comprises evaluating % time above MIC, for the drug therapy, to determine the probability of attaining a PK-PD target associated with efficacy for the patient with the infection.

In some examples, the program code obtains additional information identifying an infection. Based on the additional information, the program code generates and displays a second list comprising one or more pathogens consistent with the additional information. The program code obtains a first indication designating at least one pathogen from the second list comprising one or more pathogens from the second list. Based on at the obtaining of the least one pathogen from the second list, the program code generates a third list comprising one or more drug therapies utilized to treat the at least one pathogen. The program code obtains descriptive information relating to a second patient, the descriptive information comprising one or more data elements selected from the group consisting of: an infection acquired by the second patient, a pathogen isolated from the second patient, a creatinine clearance of the second patient, a weight of the second patient, and a height of the second patient. Based on the one or more drug therapies in the third list, the program code selects a given pharmacokinetic model. The program code applies the given pharmacokinetic model and utilizing the information relating to the second patient and the base model to determine, for each of the one or more drug therapies of the third list, a probability of attaining a PK-PD target associated with efficacy for the second patient with the infection. The program code automatically generates current rankings for each of the one or more drug therapies of the third list, by ordering each probability of attaining the PK-PD target associated with efficacy for the second patient with the infection, for each of the one or more drug therapies of the third list, for the one or more drug therapies of the third list, where the current rankings comprise a ranked list with the probability of attaining a PK-PD target associated with efficacy for the second patient with the infection for each of the one or more drug therapies of the third list, ranked in order of predicted efficacy.

In some examples, the patient-specific response modifiers are selected from the group consisting of: previous antibiotic use, age, and clearing organ function.

In some examples, the program code determining that the trigger event has occurred comprises: the program code monitoring, the program code logging of changes to the EHR system, and the program code determining, based on the logging, that the trigger event has occurred.

In some examples, the program code determining that the trigger event has occurred comprises: the program code obtaining from an application programming interface communicatively coupled with the EHR system, a notification that the trigger event has occurred.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the descriptions below, if any, are intended to include any structure, material, or act for performing the function in combination with other elements as specifically noted. The description of the technique has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described to best explain the principles of the invention and the practical application, and to enable others of ordinary skill in the art to understand the invention for various embodiments with various modifications as are suited to the particular use contemplated.

1. A computer-implemented method comprising:
determining, by one or more processors, that a trigger event related to a patient, wherein the patient record comprises an order for a current drug regimen, has occurred in an electronic health record (EHR) system communicatively coupled to the one or more processors;
based on obtaining the notification, obtaining, by the one or more processors, from one or more electronic medical records (EMRs) stored in the EHR system communicatively coupled to the one or more processors, descriptive information relating to a patient, the descriptive information comprising one or more data elements selected from the group consisting of: an infection acquired by the patient, a pathogen isolated from the patient, a creatinine clearance of the patient, a weight of the patient, and a height of the patient;
based on the one or more drug therapies, selecting a pharmacokinetic model;
applying, by the one or more processors, the pharmacokinetic model and utilizing the information relating to the patient to determine, for each of the one or more drug therapies, a probability of attaining a PK-PD target associated with efficacy for the patient with the infection;
automatically generating, by the one or more processors, rankings, for each of the one or more drug therapies, by ordering each probability of attaining the PK-PD target associated with efficacy for the patient with the infection, for each of the one or more drug therapies, for the one or more drug therapies, wherein the rankings comprise a ranked list with the probability of attaining a PK-PD target associated with efficacy for the patient with the infection for each of the one or more drug therapies, ranked in order of predicted efficacy;
determining, by the one or more processors, based on the rankings, if the current drug regimen comprises a probability above a preconfigured threshold; and
based on the determining and the rankings, generating a recommendation for a new order.

2. The computer-implemented method of claim 1, wherein the determining if the current drug regimen comprises the probability above the preconfigured threshold comprises determining that the current drug regimen comprises the probability above the preconfigured threshold, and wherein the generating comprises:
generating, by the one or more processors, a clinical note of a recommendation, wherein the clinical note of a recommendation comprises the new order, and wherein the new order and is an order for the current drug regimen; and
transmitting, by the one or more processors, the clinical node of the recommendation to the EHR system communicatively coupled to the one or more processors.

3. The computer-implemented method of claim 1, wherein the determining if the current drug regimen comprises the probability above the preconfigured threshold comprises determining that the current drug regimen does not comprise a probability above the preconfigured threshold, and wherein the generating comprises:
generating, by the one or more processors, a clinical note of a recommendation, wherein the clinical note of a recommendation comprises the new order; and
transmitting, by the one or more processors, the clinical node of the recommendation to the EHR system communicatively coupled to the one or more processors.

4. The computer-implemented method of claim 3, wherein generating the recommendation for a new order comprises:
generating, by the one or more processors, an unsigned medication order based on the ranked list, the unsigned medication order comprising the new order.

5. The computer-implemented method of claim 3, wherein generating the recommendation for a new order comprises:
transmitting, by the one or more processors, an alert to at least one user;
obtaining, by the one or more processors, a response to the alert, wherein the response comprises a selection of a designation of a drug therapy from the one or more drug therapies comprising the ranked list, and wherein the drug therapy designated comprises the new order; and
generating, by the one or more processors, an unsigned medication order comprising the new order.

6. The computer-implemented method of claim 5, wherein transmitting the alert comprises:
generating, by the one or more processors, a message comprising a link to launch a graphical user interface, wherein the one or more processors automatically display the rankings in the graphical user interface upon selection of the link by a user receiving the message; and
transmitting, by the one or more processors, the message to at least one user pre-defined to receive the message, wherein the user contact information is saved in a database communicatively coupled to the one or more processors, and wherein the response the selection of the designation of a drug therapy is performed by the user in the graphical user interface.

7. The computer-implemented method of claim 5, wherein transmitting the alert comprises:
displaying, by the one or more processors, the rankings, in a graphical user interface, wherein the response the selection of the designation of a drug therapy is performed by the user in the graphical user interface.

8. The computer-implemented method of claim 1, wherein the trigger event comprises an update to at least one field of at least one electronic medical record in the EHR system.

9. The computer-implemented method of claim 1, wherein the trigger event is selected from the group consisting of: entry of a prescription for a given antibiotic, reception of a culture with a given pathogen, and entry of data comprising additional information about an existing prescription.

10. The computer-implemented method of claim 1, further comprising:
retaining by the one or more processors, the recommendation on a memory device;
prompting, by the one or more processors, through a user interface, a user to provide data indicating an actual efficacy of the drug therapy as utilized by the patient with the infection at one or more predetermined intervals after obtaining the recommendation; and
obtaining, by the one or more processors, responsive to the prompting, the data indicating the actual efficacy of the drug therapy.

11. The computer-implemented method of claim 1, further comprising:
generating or updating, by the one or more processors, based on data comprising the data indicating the actual efficacy, a base model, wherein the base model describes a relationship between given patient response and PK-PD target attainment that accounts based on patient-specific response modifiers.

12. The computer-implemented method of claim 11, wherein the data further comprises data selected from the group consisting of: patient demographic data, clinical data, and laboratory data.

13. The computer-implemented method of claim 1, wherein the selecting the pharmacokinetic model comprises:
for each of the one or more drug therapies, determining a class for a PK-PD index;
based on determining that a drug therapy of the one or more drug therapies is in a first class, selecting a pharmacokinetic model, wherein applying the pharmacokinetic model comprises evaluating total drug exposure in a 24-hour period, for the drug therapy, to determine the probability of attaining a PK-PD target associated with efficacy for the patient with the infection; and
based on determining that a drug therapy of the one or more drug therapies is in a second class, selecting a pharmacokinetic model, wherein applying the pharmacokinetic model comprises evaluating % time above MIC, for the drug therapy, to determine the probability of attaining a PK-PD target associated with efficacy for the patient with the infection.

14. The computer-implemented method of claim 11, further comprising:
obtaining, by one or more processors, additional information identifying an infection;
based on the additional information, generating and displaying, by the one or more processors, a second list comprising one or more pathogens consistent with the additional information;
obtaining, by the one or more processors, a first indication designating at least one pathogen from the second list comprising one or more pathogens from the second list;
based on at the obtaining of the least one pathogen from the second list, generating, by the one or more processors, a third list comprising one or more drug therapies utilized to treat the at least one pathogen;
obtaining, by the one or more processors, descriptive information relating to a second patient, the descriptive information comprising one or more data elements selected from the group consisting of: an infection acquired by the second patient, a pathogen isolated from the second patient, a creatinine clearance of the second patient, a weight of the second patient, and a height of the second patient;
based on the one or more drug therapies in the third list, selecting a given pharmacokinetic model;
applying, by the one or more processors, the given pharmacokinetic model and utilizing the information relating to the second patient and the base model to determine, for each of the one or more drug therapies of the third list, a probability of attaining a PK-PD target associated with efficacy for the second patient with the infection; and
automatically generating, by the one or more processors, current rankings, for each of the one or more drug therapies of the third list, by ordering each probability of attaining the PK-PD target associated with efficacy for the second patient with the infection, for each of the one or more drug therapies of the third list, for the one or more drug therapies of the third list, wherein the current rankings comprise a ranked list with the probability of attaining a PK-PD target associated with efficacy for the second patient with the infection for each of the one or more drug therapies of the third list, ranked in order of predicted efficacy.

15. The computer-implemented method of claim 11, wherein the patient-specific response modifiers are selected from the group consisting of: previous antibiotic use, age, and clearing organ function.

16. The computer-implemented method of claim 1, wherein determining that the trigger event has occurred comprises:
monitoring, by the one or more processors, logging of changes to the EHR system; and
determining, by the one or more processors, based on the logging, that the trigger event has occurred.

17. The computer-implemented method of claim 1, wherein determining that the trigger event has occurred comprises:
obtaining, by the one or more processors, from an application programming interface communicatively coupled with the EHR system, a notification that the trigger event has occurred.

18. A computer system comprising:
a memory; and
one or more processors in communications with the memory, wherein the computer system is configured to perform a method, the method comprising:
determining, by the one or more processors, that a trigger event related to a patient, wherein the patient record comprises an order for a current drug regimen, has occurred in an electronic health record (EHR) system communicatively coupled to the one or more processors;
based on obtaining the notification, obtaining, by the one or more processors, from one or more electronic medical records (EMRs) stored in the EHR system communicatively coupled to the one or more processors, descriptive information relating to a patient, the descriptive information comprising one or more data elements selected from the group consisting of: an infection acquired by the patient, a pathogen isolated from the patient, a creatinine clearance of the patient, a weight of the patient, and a height of the patient;
based on the one or more drug therapies, selecting a pharmacokinetic model;
applying, by the one or more processors, the pharmacokinetic model and utilizing the information relating to the patient to determine, for each of the one or more drug therapies, a probability of attaining a PK-PD target associated with efficacy for the patient with the infection;
automatically generating, by the one or more processors, rankings, for each of the one or more drug therapies, by ordering each probability of attaining the PK-PD target associated with efficacy for the patient with the infection, for each of the one or more drug therapies, for the one or more drug therapies, wherein the rankings comprise a ranked list with the probability of attaining a PK-PD target associated with efficacy for the patient with the infection for each of the one or more drug therapies, ranked in order of predicted efficacy;
determining, by the one or more processors, based on the rankings, if the current drug regimen comprises a probability above a preconfigured threshold; and
based on the determining and the rankings, generating a recommendation for a new order.

19. A computer program product comprising:
a computer readable storage medium readable by a processing circuit and storing instructions for execution by the processing circuit comprising one or more processors for performing a method comprising:
determining, by the one or more processors, that a trigger event related to a patient, wherein the patient record comprises an order for a current drug regimen, has occurred in an electronic health record (EHR) system communicatively coupled to the one or more processors;
based on obtaining the notification, obtaining, by the one or more processors, from one or more electronic medical records (EMRs) stored in the EHR system communicatively coupled to the one or more processors, descriptive information relating to a patient, the descriptive information comprising one or more data elements selected from the group consisting of: an infection acquired by the patient, a pathogen isolated from the patient, a creatinine clearance of the patient, a weight of the patient, and a height of the patient;
based on the one or more drug therapies, selecting a pharmacokinetic model;
applying, by the one or more processors, the pharmacokinetic model and utilizing the information relating to the patient to determine, for each of the one or more drug therapies, a probability of attaining a PK-PD target associated with efficacy for the patient with the infection;
automatically generating, by the one or more processors, rankings, for each of the one or more drug therapies, by ordering each probability of attaining the PK-PD target associated with efficacy for the patient with the infection, for each of the one or more drug therapies, for the one or more drug therapies, wherein the rankings comprise a ranked list with the probability of attaining a PK-PD target associated with efficacy for the patient with the infection for each of the one or more drug therapies, ranked in order of predicted efficacy;
determining, by the one or more processors, based on the rankings, if the current drug regimen comprises a probability above a preconfigured threshold; and
based on the determining and the rankings, generating a recommendation for a new order.

20. A computer-implemented method comprising:
monitoring, by the one or more processors, an electronic health record (EHR) system communicatively coupled to the one or more processors;
determining, by the one or more processors, based on the listening, that an event has occurred;
determining, by the one or more processors, based on pre-configured logic, that the event comprises a covered event;
based on determining that the event is a covered event, obtaining, by the one or more processors, values to generate a simulation to determine a response to the covered event, wherein the obtaining each value of the values is selected from the group consisting of: obtaining a value from a data source and deriving the value based on obtaining one or more parameters;
simulating, by the one or more processors, based on the values a model, wherein the simulating results in a binary result indicating whether to proceed with an action or not to proceed with the action; and
based on the binary result indicating to proceed with the action, transmitting, by the one or more processors, a recommendation to proceed with the action.

21. The method of claim 20, wherein the transmitting comprises:
transmitting, by the one or more processors, an alert to at least one user; and
writing, by the one or more processors, a clinical note in the EHR.
